(19) United States
(12) Patent Application Publication
(10) Pub. No.: US 2023/0399622 A1
(43) Pub. Date: Dec. 14, 2023

(54) THERAPY FOR DEGENERATIVE DISEASE AND TISSUE DAMAGE

(71) Applicants: FUNDACIÓ CENTRE DE REGULACIÓ GENÒMICA, Barcelona (ES); INSTITUCIÓ CATALANA DE RECERCA I ESTUDIS AVANÇATS, Barcelona (ES)

(72) Inventors: Maria Pia COSMA, Barcelona (ES); Martina PESARESI, Barcelona (ES)

(21) Appl. No.: 18/032,291

(22) PCT Filed: Oct. 18, 2021
(86) PCT No.: PCT/EP2021/078855

§ 371 Date: Apr. 17, 2023

(30) Foreign Application Priority Data
Oct. 16, 2020 (EP) 20382908.0

(51) Int. Cl. C12N 5/0775; C12N 5/12; C12N 15/86; A61K 9/00; C12N 5/079; A61P 27/02

(52) U.S. Cl. CPC C12N 5/0663; C12N 5/12; C12N 15/86; A61K 9/0048; C12N 5/0622; A61P 27/02

(57) ABSTRACT

A genetically-modified stem cell comprises an exogenous nucleic acid encoding a chemokine receptor. The exogenous nucleic acid is operably linked to at least one promoter and/or enhancer sequence for expression of the chemokine receptor in the genetically-modified stem cell. The chemokine receptor may be selected from Ccr5, Cxcr6, Ccr1, Cxcr2 and/or Ccr3. The genetical-ly-modified stem cell may be used in a method for the treatment of tissue damage and/or degenerative disease, such as an eye disease or disorder. A modified viral vector packaging a recombinant viral-based genome, for use in a method of treating a subject suffering from tissue damage and/or degenerative disease, such as an eye disease or disorder is also described. Methods for treating tissue damage and/or degenerative disease, such as eye diseases or disorders, and pharmaceutical compositions for use in such methods are also described.

### FIELD OF THE INVENTION

This invention relates to therapeutic compositions, uses of compositions and methods for treatment of degenerative disease and tissue damage, in particular, diseases and disorders of the eye. In particular, the invention relates to genetically-modified cells, such as stem cells, expressing exogenous chemokine receptors for use in the treatment of damaged tissues and degenerative diseases, in particular, retinal diseases, disorders or damage including various retinopathies.

### BACKGROUND OF THE INVENTION

Tissue damage and cell loss, by trauma, degenerative disease, autoimmunity or any other cause, underlie pathology in many contexts. Tissue or organ functionality can be reduced by the loss of cells, and intrinsic responses to the pathology can cause further damage. In many cases, the adult organism cannot replenish the lost cells, and even attempts to restore the lost cells, for example through stem cell therapy (SCT) can lack efficacy, due at least in part to the inability of the transplanted stem cells to recreate the complex tissue organisation and/or cell-cell connections present in healthy conditions but also to their inability to efficiently migrate and integrate in the tissue. This is therefore particularly applicable to tissues with low capacity for self-regeneration, and/or which possess complex histology, such as the nervous system and the skin.

As an example, retinopathies are currently incurable. They inevitably lead to visual disabilities and, in most cases, blindness. Worryingly, the number of people affected by retinopathies is estimated to increase dramatically over the next few decades, due both to growth and aging of the population.

Stem cell therapy (SCT) has been proposed as a potential solution to the incurability of degenerative retinal conditions. Therapeutically, stem cells (SCs) transplanted into the eye can exert beneficial effects in one of two ways. First, they can release biologically active molecules. This paracrine effect has potent neuroprotective and anti-inflammatory properties; it strongly promotes the survival, proliferation and self-repair of endogenous cells (Baraniak & McDevitt (2010), Regenerative Medicine, 5(1):121-43). Second, SCs can generate new tissue-specific cells, thereby replacing lost or damaged ones. To facilitate this process, SCs can be differentiated towards specific desired progenitor types in vitro, prior to transplantation (MacLaren et al., (2006), Nature, 444(7116):203-7).

Although promising, SCTs require further development and optimisation. In particular, cells transplanted into the eye do not efficiently migrate and integrate into the retina (Inoue et al. (2007), Experimental Eye Research, 85(2):234-41; Jiang et al. (2010), Molecular Vision, 16:983-990), especially following intravitreal injection (Johnson et al. (2009), Eye, 23(10):1980-1984).

The present invention seeks to overcome or at least alleviate one or more of the problems found in the prior art. In particular, the present invention aims to address problems related to inadequate migration and/or integration of transplanted cells.

### SUMMARY OF THE INVENTION

It is an objective of the present invention to provide an efficient therapy for tissue damage and/or degenerative disease, such as eye diseases and disorders as mentioned above, particularly of eye diseases and disorders caused by a degeneration of the retina, which avoid these problems and which allow to provide an efficient long term treatment without the need of frequently repeated drug administration and/or the risk of undesired side effects.

According to one aspect of the disclosure, there is provided a genetically-modified stem cell comprising an exogenous nucleic acid encoding a chemokine receptor, wherein the exogenous nucleic acid is operably linked to at least one promoter and/or enhancer sequence for expression of the chemokine receptor in the genetically-modified stem cell. In embodiments, the genetically-modified stem cell comprises one or more exogenous nucleic acid, wherein each of the one or more exogenous nucleic acid encoding a chemokine receptor selected from a group consisting of: (i) Ccr5, Cxcr6, Ccr1, Cxcr2 and/or Ccr3; (ii) Ccr5, Cxcr6, Ccr1 and/or Cxcr2; or (iii) Ccr5 and/or Cxcr6. In another aspect, however, the invention comprises a genetically-modified stem cell capable of causing expression (or increased expression) of an endogenous chemokine gene as described elsewhere herein.

Suitably, the genetically-modified stem cell is selected from the group consisting of: pluripotent stem cells, multipotent stem cells and unipotent stem cells. For example, the stem cell may particularly be a mesenchymal stem cell (MSC); or a photoreceptor precursor cell. The cell-type may suitably be selected according to the intended treatment and/or the target therapeutic (cellular) region. The intended treatment and/or target region may be that of the eye, and especially the retina. For example, photoreceptor precursor cells may be particularly suited for integration into the outer nuclear layer (ONL), whereas mesenchymal stem cells may be particularly suited for integration into and/or cooperation with the ganglion cell layer (GCL).

In some embodiments, the exogenous nucleic acid is integrated into the genome of the genetically-modified stem cell.

Typically, the exogenous nucleic acid is part of a proviral sequence. The proviral sequence may suitably be selected from the group consisting of: retroviruses (such as influenza, simian immunodeficiency virus (SIV), human immunodeficiency virus (HIV), lentivirus, and Moloney murine leukaemia); adenoviruses; adeno-associated viruses (AAV); sendai virus (SeV); herpes simplex virus (HSV); and chimeric viruses.

For example, more suitably, the proviral sequence is selected from: (i) an adeno-associated virus (AAV) sequence; (ii) a sendai virus (SeV) sequence; (iii) an AAV vector sequence selected from the group consisting of: AAV1, AAV2, AAV3, AAV4, AAV5, AAV6, AAV6.2, AAV7, AAV8, AAV9, rhlO, AAVrh64RI, AAVrh64R2 and rh8 or variants thereof; or (iv) an AAV vector sequence selected from AAV2/1 and AAV2/9 viral subtypes.

In some embodiments, the exogenous nucleic acid is not integrated into the genome of the genetically-modified stem cell. In some embodiments, the exogenous nucleic acid is RNA, typically mRNA.

In some embodiments, the genetically-modified stem cell is capable of secreting one or more endogenous proteins or peptides as paracrine factors. Optionally, the paracrine factor is selected from VEGF, IL6, IL8, GDNF, NT3, NeuroD1 and/or MCP1.

In some embodiments, the genetically-modified stem cell comprises an exogenous nucleic acid encoding a selectable or screenable marker which is operably linked to at least one promoter and/or enhancer sequence for expression of the selectable or screenable marker in the genetically-modified stem cell.

In another aspect of the disclosure, there is provided a pharmaceutical composition comprising the genetically-modified stem cell according to the disclosure. In some embodiments of this aspect, the pharmaceutical composition is formulated as an injectable composition. Particularly, the pharmaceutical composition may be formulated for intraocular administration; for example, for intravitreal administration and/or for subretinal administration.

Genetically-modified stem cells according to the disclosure, or pharmaceutical compositions according to the disclosure may be for use in a method for the treatment of an eye disease or disorder; such as a retinopathy and/or damaged retina. In embodiments, the eye disease or disorder is caused by a degeneration of the retina (retinal disease), such as: retinitis pigmentosa (RP), macular degeneration (MD), age related macular degeneration (AMD or ARMD), macula edema due to other reasons, retinal vessel occlusions, ischaemic injury, diabetic retinopathy, glaucoma and other optic neuropathies, etc., and conditions associated therewith; Bassen-kornzweig syndrome, choroideremia, gyrate atrophy, Refsum syndrome, Usher syndrome, color blindness, blue cone monochromacy, achromatopsia, incomplete achromatopsia, oligocone trichromacy, Stargardt's Disease, Bardet-Biedl syndrome, Bornholm eye disease, Best's Disease and Leber's congenital amaurosis.

Genetically-modified stem cells according to the disclosure, or pharmaceutical compositions according to the disclosure may be for use in a method for the treatment of tissue damage and/or of a degenerative disease. In embodiments, the degenerative disease may be a neurodegenerative disease, such as Parkinson's Disease (PD), Huntington's disease (HD), Alzheimer's Disease (AD), frontotemporal dementia (FTD) and amyotrophic lateral sclerosis (ALS). In embodiments, the stem cells or compositions may be for use in the treatment of damage to nervous tissue, such as the spinal cord or brain, for example resulting from ischaemic injury. In embodiments, the stem cells or compositions may be for use in the treatment of myocardial damage, for example resulting from myocardial infarction. In embodiments, the stem cells or compositions may be for use in the treatment of disorders of the skin, for example epidermolysis bullosa or radiation-induced oral mucositis. In embodiments, the stem cells or compositions may be for use in the treatment of disorders of the bones or joints, for example arthritis, arthrosis, pseudoarthrosis, infections, resection or trauma. In embodiments, the stem cells or compositions may be for use in the treatment of disorders of the lung, such as resulting from smoking, surgery, trauma, or infection.

In some embodiments of these aspects, the genetically-modified cell or the pharmaceutical composition may be for use in a method for treatment, which comprises injection of the genetically-modified cell or pharmaceutical composition into or proximal to the tissue or target region for treatment, for example, into the brain or heart. The method may comprise intradiscal administration into the spine. The method may comprise intraocular injection of the genetically-modified cell or pharmaceutical composition into the intravitreal space or into the subretinal space. Optionally: (i) wherein the genetically-modified cell is an MSC, and wherein the method comprises intraocular injection of the genetically-modified MSC or pharmaceutical composition comprising the genetically-modified MSC into the intravitreal space such that the genetically-modified MSC integrates into the ganglion cell layer (GCL) and/or cooperates with the ganglion cell layer (GCL) to repair and/or protect and/or increase the thickness of the GCL; and/or (ii) wherein the genetically-modified cell is a photoreceptor precursor cell, and wherein the method comprises intraocular injection of the genetically-modified photoreceptor precursor cell or pharmaceutical composition comprising the photoreceptor precursor cell into the subretinal space such that the photoreceptor precursor cell integrates into the outer nuclear layer (ONL) and/or cooperates with photoreceptor cells of the outer nuclear layer (ONL) to repair and/or protect and/or increase the thickness of the ONL.

In another aspect of the disclosure, there is provided a modified viral vector packaging a recombinant viral-based genome, for use in a method for treating a subject suffering from tissue damage and/or a degenerative disease, the method comprising: administering to the subject one or more of the modified viral vectors packaging a recombinant viral-based genome, wherein the recombinant viral-based genome comprises a cDNA insert encoding an exogenous chemokine receptor polypeptide; wherein, in use, the one or more of the modified viral vectors infects and causes increased expression of the chemokine receptor in cells of the subject. In embodiments, the subject is suffering from an eye disease or disorder, and the one or more of the modified viral vectors infects and causes increased expression of the chemokine receptor in retinal cells. In another aspect, there is provided a modified viral vector packaging a recombinant viral-based genome for use in a method for treating a subject suffering from tissue damage and/or a degenerative disease, the method comprising: administering to the subject one or more of the modified viral vectors packaging a recombinant viral-based genome, wherein the recombinant viral-based genome comprises a cDNA insert encoding an exogenous chemokine polypeptide; wherein, in use, the one or more of the modified viral vectors infects and causes increased expression of the chemokine in cells of the subject. In embodiments, the subject is suffering from an eye disease or disorder, and the one or more of the modified viral vectors infects and causes increased expression of the chemokine in retinal cells. Optionally, the cDNA insert may be operably linked to at least one promoter and/or enhancer sequence for expression of the exogenous chemokine receptor polypeptide in retinal cells of the subject. In various embodiments, the viral vector is an adeno-associated virus (AAV), or a sendai virus (SeV). In some embodiments, the chemokine receptor is selected from one or more chemokine receptor selected from a group consisting of: (i) Ccr5, Cxcr6, Ccr1, Cxcr2 and/or Ccr3; (ii) Ccr5, Cxcr6, Ccr1 and/or Cxcr2; or (iii) Ccr5 and/or Cxcr6. In some embodiments, the chemokine is a chemokine as disclosed herein; in particular, one that it recognized by one or more of the chemokine receptors disclosed herein; for example, one or more of CCL3, CCL5, CCL7, CCL23, CCL4, CCL3L1, CCL11, CCL26, CCL13, IL8, CXCL1, CXCL2, CXCL5, and/or CCL16.

In another aspect there is provided a method for treating or alleviating an eye disease or disorder (such as a retinopathy and/or damaged retina), in a subject. The method of this aspect may comprise: introducing into the subject's eye a genetically-modified stem cell according to the disclosure; and/or a modified viral vector according to the disclosure.

Other aspects and embodiments of the invention will be apparent from reading this disclosure; and particularly having regard to the Clauses and Claims provided herein below.

It will be appreciated that any features of one aspect or embodiment of the disclosure (e.g. according to any one or more of the Clauses and Claims provided herein below) may be combined with any combination of features in any other aspect or embodiment of the disclosure, unless otherwise stated, and such combinations fall within the scope of the claimed invention.

### BRIEF DESCRIPTION OF THE DRAWINGS

One or more embodiments of the invention will now be described, byway of example only, with reference to the accompanying drawings, in which:

FIG. 1: Retinal damage is associated with an acute peak in 111-#3 expression. qRT-PCR of II1-β levels in the (A) NMDA-damaged retina (24 hours post-injection (hpi), 48 hpi, 4 days post-injection (dpi), 7 dpi, 4 weeks post injection (wpi)) and in the (B) rd10 retina (at post-natal day (P)14, P18, P22, adult). Transcript levels are expressed as fold-changes to control (PBS-injected or P14) retina. Data are presented as mean±SEM from n≤3 independent experiments. One Way Anova was used for statistical analysis. (C) Experimental scheme of chemotactic assays with murine samples. Wild-type (WT) mice received intravitreal injection of NMDA to induce retinal degeneration; the contralateral eye was injected with PBS, as a control. PBS/NMDA-injected animals were sacrificed 24 hpi. Rd10 mice (and their age-matched WT controls) were instead sacrificed at P18. Eyecups were then cultured for 24 hours in serum-free (SF) medium. At the moment of the assay, a suspension of 2×10⁵ MSC in SF medium was seeded in the upper chamber. Following incubation, non-migrated cells were removed. Migrated cells stuck in the porous membrane of the transwell were stained with DAPI, imaged and quantified. Representative DAPI-stained fields from PBS vs NMDA (D), and from WT vs rd10 (E) transwell assays. (F) Representative DAPI-stained fields from healthy, NMDA and RP transwell assays performed with human samples. (G) Experimental scheme of chemotactic assays with human samples. The central part of the retina was divided into quarters. For each of the experiments, a quarter was cultured in control medium, while another one was cultured in medium containing NMDA (1 mM). Similarly, a quarter from a healthy control and a quarter from a RP retina were cultured in parallel. After 24 hours, the conditioned medium was collected and used to perform chemotactic assays.

FIG. 2: Ccr1, Ccr3, Ccr5 and Cxcr6 mediate chemoattraction of MSCs towards media conditioned by degenerating retina. Quantification of migrated MSCs towards the conditioned media from either (A) PBS- vs NMDA-injected, or (B) WT vs rd10P18 eyecups. Number of migrated cells is expressed as fold-change relative to control samples (PBS-injected or WT). Data are presented as mean±SD (n 3). Mann-Whitney test was used for statistical analysis. (C) Quantification of migrated MSCs towards the conditioned media from human retina cultured in control medium, NMDA-containing (NMDA) medium, or from RP-affected retina. Number of migrated cells is expressed as fold-change relative to control retina. Data are presented as mean±SD (n 3). Mann-Whitney test was used for statistical analysis. CC and CXC inflammatory chemokine profiles from either (D) PBS- vs NMDA-injected (24 hpi), or (E) WT vs rd10 P18 retinal lysates. Pixel intensity is directly proportional to the total amount of protein in sample. Orange arrows point at representative chemokines whose abundance is increased in degenerating retina (NMDA or rd10), relative to controls (PBS or WT). Data are presented as mean±SD from n=2 independent experiments. qRT-PCR of CC and CXC inflammatory chemokines from (F) NMDA-damaged (24 hpi) and (G) rd10 (P18) retina. Transcript levels are expressed as fold-change relative to control (PBS-injected or WT) retina. Data are presented as mean±SD (n≥3). Mann-Whitney test was used for statistical analysis. (H) Scheme summarising the interactions among chemokine receptors and ligands of interest. Ccr1, Ccr3 and Ccr5 are bound by Ccl5, Ccl6 and Ccl12. Cxcr2 is bound by Cxcl1, Cxcl2 and Cxcl5. Cxcr6 is bound by Cxcl16. Quantification of migrated MSCs towards the conditioned media from either (1) NMDA-damaged (24 hpi) or (J) rd10 (P18) retina, in the presence of the inhibitors of Ccrs (Ccr1, Ccr3 and Ccr5-iCcrs), Cxcr2 (iCxcr2), Cxcr6 (iCxcr6) or Ccrs+Cxcrs (Ccr1, Ccr3, Ccr5-iCcrs; Cxcr2 and Cxcr6-iCxcrs). Number of migrated cells is expressed as fold-change to control (vehicle—DMSO). Data are presented as Min to Max boxes (with line at median) from n≥3 independent experiments. Mann-Whitney test was used for statistical analysis.

FIG. 3: Retina degeneration is associated with time-dependent inflammation and increased expression of specific CC and CXC inflammatory chemokines. (A) Experimental scheme indicating analysed time-points for PBS- and NMDA-injected eyes (24 hpi, 48 hpi, 4 dpi, 7 dpi, 4 wpi). Time course analysis of II1-β and either of CC (B) or CXC (C) chemokines expression in NMDA-damaged retina. Transcript levels are expressed as fold-change to control (PBS-injected) retina. Data are presented as mean from n≥3 independent experiments. (D) Experimental scheme indicating analysed time-points for WT and rd10 retina (P14, P18, P22, adult). Time course analysis of II1-β and either of CC (E) or CXC (F) chemokines expression in rd10 retina. Transcript levels are expressed as fold-change to P14 retina. Data are presented as mean from n≥3 independent experiments. (G) qRT-PCR of pro-inflammatory cytokines IL1-β and TNF-α in healthy, NMDA-damaged and RP human retina, cultured for 24 hours. Transcript levels are expressed as fold-change to the healthy controls. Data are presented as mean±SD from n≥3 (NMDA damaged) or n=2 (RP) independent experiments. Mann-Whitney test was used for statistical analysis. (H) qRT-PCR of CC and CXC chemokines levels in healthy, NMDA-damaged and RP human retina, cultured for 24 hours. Transcript levels are expressed as fold-change to the healthy controls. Data are presented as mean±SD from n≥3 (NMDA-damage) or n=2 (RP) independent experiments. Mann-Whitney test was used for statistical analysis. (1) Representative DAPI-stained fields from transwell assays assessing migration of mMSCs towards either NMDA-damaged (top) or rd10 (bottom) retina, in the presence of DMSO (vehicle), iCxcr2, iCcrs (Ccr1, Ccr3, Ccr5), iCxcr6 or iCcrs (Ccr1, Ccr3, Ccr5)+iCxcrs (iCxcr2+iCxcr6).

FIG. 4: Characterization of OE-MSC lines. (A) qRT-PCR of endogenous Ccr1, Ccr3, Ccr5, Cxcr2, Cxcr3, Cxcr6 and Thy (Cd90) expression in mMSCs. Transcript levels are normalised to Gapdh expression. Data are presented as mean±SD from n=3 independent experiments. (B) Lentiviral constructs used to infect mMSCs. Constitutive EF1a and SV40 promoters drive expression of the HA-tagged receptors (i.e. Ccr1, Ccr3, Ccr5, Cxcr2, Cxcr3 and Cxcr6, or empty control) and of the eGFP marker respectively. (C) qRT-PCR showing expression of the Ccr1, Ccr3, Ccr5, Cxcr2, Cxcr3 and Cxcr6 genes in the corresponding mMSC-OE lines. Transcript levels are expressed as fold-change to WT-MSC control. Data are presented as mean±SD from n=3 independent experiments. Mann-Whitney test was used for statistical analysis. (D) Representative immunofluorescence staining of OE-MSC lines (Ccr1, Ccr3, Ccr5, Cxcr2, Cxcr3 and Cxcr6) to verify expression of the eGFP (green) and of the HA (red) tags. (F) Representative FACS gating strategy. From left to right: side scatter area vs forward scatter area; forward scatter height vs forward scatter area, to exclude cell-cell aggregates; Pacific Blue area vs side scatter area to exclude DAPI+ and keep only living cells in the analysis; example of an NMDA-damaged retina that had not been transplanted with GFP+MSCs, to set gate for GFP+cells. (G) Representative FACS plots from MSC-WT, MSC-Ccr5, MSC-Cxcr2 and MSC-Cxcr6 transplanted into NMDA-damaged retina.

FIG. 5: OE-MSCs display increased chemotactic response towards the media conditioned by damaged retina and improved migration following transplantation in the vitreous. (A) Cell-surface FACS analysis of Ccr1, Ccr5, Cxcr2, Cxcr3 and Cxcr6 receptors of the corresponding OE-MSC lines, and of WT-MSCs as a control. Results are expressed as percentage of positive cells. Data are presented as mean±SD from n≥3 independent experiments. Two-tailed Student's T-test was used for statistical analysis. (B) Quantification of migrated MSCs towards a concentration of 50 ng ml⁻¹ of Ccl5 (Ccr1-MSC, Ccr3-MSC and Ccr5-MSC), Cxcl1 (Cxcr2-MSC), Cxcl10 (Cxcr3-MSC) or Cxcl16 (Cxcr6-MSC), from transwell-based assays. Number of migrated cells is expressed as fold-change to control (WT-MSC). Data are presented as mean±SD from n≥3 independent experiments. Mann-Whitney test was used for statistical analysis. Quantification of migrated MSCs overexpressing the chemokine receptors towards the conditioned media from either (C) NMDA-injected or (D) rd10 retina, in transwell-based assays. Number of migrated cells (OE-MSCs) is expressed as fold-change to control (WT-MSC). Data are presented as mean±SD from n≥3 independent experiments. Mann-Whitney test was used for statistical analysis. Quantification of migrated MSCs towards the conditioned media from either (E) NMDA-damaged or (F) RP human retina, in transwell-based assays. Number of migrated cells is expressed as fold-change to control (WT-MSC). Data are presented as mean±SD from n≥3 independent experiments. Mann-Whitney test was used for statistical analysis. (G) Experimental scheme. Eyes were damaged via NMDA-injection 12 hours prior to transplantation of either WT-MSCs or OE-MSCs (Cxcr2-MSCs, Ccr5-MSCs or Cxcr6-MSCs). FACS analysis was performed 4 days after the transplant (4 days post-injection (dpi)). (H) FACS-based quantification of GFP⁺ MSCs (WT-, Cxcr2-, Ccr5-, or Cxcr6-) in transplanted retina, 4 dpi. Results are expressed as percentage of total retinal cells. Data are presented as Min. to Max. boxes (with line at median) from n≥3 independent experiments. Two-tailed Student's T-test was used for statistical analysis.

FIG. 6: Characterisation of Ccr5-Cxcr6-MSCs. (A) Scheme of lentiviral plasmids used to infect MSCs. Constitutive EF1a and SV40 promoters drive expression of the HA-tagged receptors (i.e. Ccr5 or Cxcr6) and of the markers (i.e. eGFP or Hygromycin) respectively. Ccr5-Cxcr6 double positive cells were isolated by FACS-sorting for eGFP and applying hygromycin selection. qRT-PCR showing expression of either the (B) Ccr5 or the (C) Cxcr6 genes in the Ccr5-Cxcr6 double positive MSCs and in the corresponding single expressing OE-MSC lines (i.e. Ccr5-MSC or Cxcr6-MSC). Transcript levels are expressed as fold-change to GFP+Empty- (WT-) MSC control. Data are presented as mean±SD from n=3 independent experiments. Two-tailed Student's T-test was used for statistical analysis. (D) Representative immunofluorescence staining of Ccr5-Cxcr6-MSCs to verify expression of the eGFP (green) and of the HA (red) tags. Scale bar=100 μm. (E) Flow cytometry analysis comparing the levels of the indicated surface markers (CD44, CD90, CD45, CD34, Sca-I and CD11b) on WT-, GFP- and dOE-MSCs. qRT-PCR showing expression of (F) CyclinD1, (G) Bdnf, (H) Ngf, (1) Pdgf-α and (J) Ctnf genes in GFP- and dOE-MSCs. Transcript levels are normalised to Gapdh expression. Data are presented as mean±SD from n=3 independent experiments. Two-tailed Student's T-test was used for statistical analysis. (K) Profile of neurotrophic factors secreted in SF medium over 24 hours by GFP- and dOE-MSCs. Pixel intensity is directly proportional to the total amount of protein in sample. No statistically significant changes were detected. Data are presented as mean±SD from n=2 independent experiments. (L) Quantification of migrated MSCs (Ccr5, Cxcr6, or Ccr5-Cxcr6) towards a concentration of 50 ng/ml of Ccl5 and Cxcl16, from transwell-based assays. Number of migrated cells is expressed as fold-change to control (WT-MSC). Data are presented as mean±SD from n≥3 independent experiments. Mann-Whitney test was used for statistical analysis. (M) Representative FACS plots from MSC-WT, MSC-Ccr5, MSC-Cxcr6 and MSC-Ccr5-Cxcr6 transplanted into NMDA-damaged retina.

FIG. 7: Over-expression of Ccr5 and Cxcr6 improves migration and in vivo integration of transplanted MSCs. Quantification of migrated MSCs (Ccr5-, Cxcr6, or Ccr5-Cxcr6) towards the conditioned media from either (A) NMDA-injected or (B) rd10 retina, from transwell-based assays. Number of migrated cells is expressed as fold-change to control (WT-MSC). Data are presented as mean±SD (n 3). Mann-Whitney test was used for statistical analysis. (C) FACS-based quantification of GFP⁺ MSCs (WT-, Ccr5-, Cxcr6-, or Ccr5-Cxcr6-) in transplanted retina, 4 dpi. Results are expressed as percentage of total retinal cells. Data are presented as mean±SD (n 3). Two-tailed Student's T-test was used for statistical analysis. Representative fields from WT- vs dOE-MSCs flat mounts, prepared from either (D) NMDA-injected or (F) rd10 retina, and stained against GFP. Scale bar=100 μm. Either the GCL/INL (D) or the ONL (F) were imaged. Quantification of GFP⁺ cells counted in at least 3 fields per animal, in both (E) NMDA-injected and (G) rd10 retina (n 3). Data are presented as mean±SD. Two-tailed Student's T-test was used for statistical analysis.

FIG. 8: A percentage of transplanted MSCs might be phagocytosed or undergo cell fusion. (A) Field of view from an undamaged area of retinal flat mount prepared from an NMDA-injected eye that had been transplanted with dOE-MSCs, 3 weeks post-injection (3 wpi). The tissue was stained for GFP (green), βIII-tubulin (red), and DAPI (blue). Scale bar=50 μm. (B) Imaging of RPE cells co-cultured with GFP-MSC derived apoptotic bodies for 0 (RPE), 3, 12 and 48 hours. (C) Flow cytometry analysis of RPE cells co-cultured with GFP-MSC-derived apoptotic bodies for 0 (RPE), 1, 3, 6, 12, 48 and 72 hours. (D) Representative FACS plots for RPE (control), 3 hour and 48 hour samples. Based on the RPE control, the gate for GFP positive, actively phagocytic, RPEs (total) was defined; within such gate, the population of GFP-high RPE cells (GFP-high) was analysed. (E) Quantification of GFP+-DsRed+double positive cells from DsRed+retinal flat mounts wpi, expressed as a percentage over the total GFP+cells in the field of view. Data are presented as mean±SD (n=3). Two-tailed Student's T-test was used for statistical analysis. (F, G) Representative field of view from retinal flat mounts prepared from an NMDA-damaged DsRed+eye that had been transplanted with dOE-MSCs (3 wpi). The tissue was stained for GFP (green), DsRed (red) and DAPI (blue). Scale bar=50 μm. Inserts at the bottom-left corner show higher magnification of the regions in the white square and include one representative heterokaryon (scale bar=5 μm).

FIG. 9: Functional rescue of the retinal degeneration upon transplantation of dOE-MSCs. (A) Representative H&E staining of 2 months-old rd10 retina that had been transplanted with PBS (left), WT-MSCs (center) or dOE-MSCs (right), 3 weeks post-injection (wpi). GCL=ganglion cell layer; INL=inner nuclear layer; ONL=outer nuclear layer). (B) Quantification of photoreceptor rows in retina that had been transplanted with PBS, WT-MSCs or dOE-MSCs, 3 wpi. Results are presented as fold-change to control (PBS). Data are expressed as mean±SD (n=3). Mann-Whitney test was used for statistical analysis. (C) Statistical analysis of ERG responses of 2 months-old rd10 mice that had received transplantation of MSCs (either WT or dOE) in one eye, and of vehicle alone (PBS) in the other eye, 3 wpi. Data are presented as mean±SD (n=2) of the difference between the amplitudes of the waves recoded in the MSC-injected eye and of those recorded in the contralateral (PBS-injected) eye (Δ_{μv}). Two-tailed Student's T-test was used for statistical analysis. (D) Representative field of view from a retinal flat mount prepared from an NMDA-damaged eye at 3 weeks post-injection (3 wpi) that had been transplanted with dOE-MSCs. The tissue was stained for GFP (green), βIII-tubulin (red), and DAPI (blue). Scale bar=20 μm. Inserts at the bottom-right corner show higher magnification of the regions in the white square (scale bar=10 μm). Quantification of GFP⁺-βIII-tubulin⁺ double positive cells from retinal flat mounts, expressed either as number of cells per field of view (E), or as percentage of GFP⁺-BIII⁺-tubulin cells over the total GFP⁺ cells in the field of view (F). Data are presented as mean±SD (n=3). Two-tailed Student's T-test was used for statistical analysis. (G) Representative field of view from a retinal flat mount prepared from an NMDA-damaged eye at 3 wpi that had been transplanted with dOE-MSCs. The tissue was stained for GFP (green), Neun (red), Smi-32 (magenta) and DAPI (blue). Scale bar=50 μm. Inserts at the bottom-right corner show higher magnification of the regions in the white square (scale bar=5 μm). (H) Representative field of view from a retinal flat mount prepared at 3 wpi from an rd10 eye that had been transplanted with dOE-MSCs. The tissue was stained for GFP (green), Rhodopsin (red), and DAPI (blue). Scale bar=20 μm. Inserts at the bottom-right corner show higher magnification of the regions in the white square (scale bar=20 μm). Quantification of GFP⁺-Rhodopsin⁺ double positive cells from retinal flat mounts, expressed either as number of cells per field of view (1), or as percentage of GFP⁺—Rhodopsin⁺ cells over the total GFP⁺ cells in the field of view (J). Data are presented as mean±SD (n=3). Two-tailed Student's T-test was used for statistical analysis. (K) RT-PCR of Pde6b expression in retina harvested from rd10 retina that had been transplanted at P18 either with PBS or dOE-MSCs. Data are presented as mean±SD (n=3). Two-tailed Student's T-test was used for statistical analysis.

FIG. 10: Transplanted MSCs integrate into the NMDA-damaged retina and express neural markers 3 weeks post-injection. (A) Representative retinal section prepared from an NMDA-damaged eye that had been transplanted with dOE-MSCs, 3 wpi. The tissue was stained for GFP (green), pill-tubulin (red) and DAPI (blue). Scale bar=20 μm. Inserts at the bottom-left corner show higher magnification of the regions in the white square (scale bar=5 μm). (B) Representative field of view from retinal flat mounts prepared from an NMDA-damaged eye that had been transplanted with dOE-MSCs (3 wpi). The tissue was stained for GFP (green), βIII-tubulin (red), phospho-Histone H3 (Ser10) (white) and DAPI (blue). Scale bar=50 μm. Inserts at the bottom-left and upper-right corners show higher magnification of the regions in the white and yellow squares respectively (scale bar=5 μm). (C) Representative field of view from retinal flat mounts prepared from an NMDA-damaged eye that had been transplanted with dOE-MSCs (3 wpi). The tissue was stained for GFP (green), calbindin (magenta) and DAPI (blue). Scale bar=20 μm. Inserts at the bottom-left corner show higher magnification of the regions in the white square (scale bar=5 μm). (D) Representative retinal section prepared from an NMDA-damaged eye that had been transplanted with dOE-MSCs, 3 wpi. The tissue was stained for GFP (green), Neun (red) and DAPI (blue). Scale bar=50 μm. Inserts at the bottom-left corner show higher magnification of the regions in the white square (scale bar=10 μm). (E) Representative field of view from retinal flat mounts prepared from an NMDA-damaged eye that had been transplanted with dOE-MSCs (3 wpi). The tissue was stained for GFP (green), CD90 (red) and DAPI (blue). Scale bar=50 μm. Inserts at the bottom-right corner show higher magnification of the regions in the white square (scale bar=5 μm). (F) Representative field of view from retinal flat mounts prepared from an NMDA-damaged eye that had been transplanted with dOE-MSCs (3 wpi). The tissue was stained for GFP (green), GFAP (magenta) and DAPI (blue). Scale bar=50 μm. Inserts at the bottom-left corner show higher magnification of the regions in the white square (scale bar=5 μm). (G) Quantification of GFP+-GFAP+double positive cells from NMDA-damaged retinal flat mounts 3 wpi, expressed as a percentage over the total GFP+cells in the field of view. Data are presented as mean±SD (n=3). Two-tailed Student's T-test was used for statistical analysis.

FIG. 11: Transplanted MSCs partially integrate into the host tissue and express photoreceptor markers 3 weeks post-injection. (A) Representative field of view from retinal flat mounts prepared from an rd10 eye that had been transplanted with dOE-MSCs (3 wpi). The tissue was stained for GFP (green), CD90 (red) and DAPI (blue). Scale bar=50 μm. Inserts at the bottom-left corner show higher magnification of the regions in the white square (scale bar=5 μm). (B) Orthogonal view from a retinal flat mount that had been transplanted with dOE-MSCs, 3 wpi. The tissue was stained for GFP (green), rhodopsin (red), and DAPI (blue). The white arrow points at a GFP+cells that appears to be physically located within the same layer as the rhodopsin+(orange arrow). (C, D) Representative retinal section prepared from an rd10 eye that had been transplanted with either GFP- (up) or dOE-(down) MSCs, 3 wpi. The tissue was stained for GFP (green), rhodopsin (C) or recoverin (D) (red), and DAPI (blue). Scale bar=50 μm. Inserts at the bottom corner show higher magnification of the regions in the white square (scale bar=20 μm (C); or 10 μm (D)).

### DETAILED DESCRIPTION OF THE INVENTION

All references cited herein are incorporated by reference in their entirety. Unless otherwise defined, all technical and scientific terms used herein have the same meaning as commonly understood by one of ordinary skill in the art to which this invention belongs (e.g. in cell culture, molecular genetics, nucleic acid chemistry and biochemistry).

Unless otherwise indicated, the practice of the present invention employs conventional techniques of chemistry, molecular biology, microbiology, recombinant DNA technology, chemical methods, pharmaceutical formulations and delivery and treatment of animals, which are within the capabilities of a person of ordinary skill in the art. Such techniques are also explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

In order to assist with the understanding of the invention several terms are defined herein.

Therapeutic Uses, Methods and Treatments:

In various aspects and embodiments, the present invention provides therapeutic compositions for use in the treatment of tissue damage and/or degenerative diseases, in particular of eye diseases or disorders, as well as therapeutic methods for the treatment of such diseases. Suitable conditions to be treated include neurodegenerative diseases, such as Parkinson's Disease (PD), Huntington's disease (HD), Alzheimer's Disease (AD), frontotemporal dementia (FTD) and amyotrophic lateral sclerosis (ALS); damage to nervous tissue, such as the spinal cord or brain; myocardial damage, for example resulting from myocardial infarction; disorders of the skin, for example epidermolysis bullosa or radiation-induced oral mucositis; disorders of the bones or joints, such as arthritis, arthrosis, pseudoarthrosis, infections, resection or trauma; disorders of the lung, such as resulting from smoking, surgery, trauma, or infection; or autoimmune or inflammatory diseases.

Suitable eye diseases or disorders are diseases and disorders caused by a degeneration of the retina (retinal disease), such as, among other retinal diseases, retinitis pigmentosa (RP), macular degeneration (MD), age related macular degeneration (AMD or ARMD), macula edema due of other reasons, retinal vessel occlusions, diabetic retinopathy, glaucoma and other optic neuropathies, etc., and the treatment or prevention of conditions associated therewith. In embodiments, the retinal disease may particularly affect cone photoreceptors and/or rod photoreceptors. Such retinal diseases may affect the cone photoreceptors and/or rod photoreceptors directly or indirectly. Further retinal diseases that may be treated in relation to disorders in cone photoreceptors also include Bassen- kornzweig syndrome, choroideremia, gyrate atrophy, Refsum syndrome, Usher syndrome, color blindness, blue cone monochromacy, achromatopsia, incomplete achromatopsia, oligocone trichromacy, Stargardt's Disease, Bardet-Biedl syndrome, Bornholm eye disease, Best's Disease and Leber's congenital amaurosis.

As used herein, the term ‘treatment’ or ‘treat’ refer to both prophylactic or preventive treatment as well as curative or disease modifying treatment, including treatment of subject or patient at risk of contracting the disease or suspected to have contracted the disease as well as patients who are ill or have been diagnosed as suffering from a disease or medical disorder, and includes suppression of clinical relapse. The treatment may be administered to a subject having a disease or medical disorder or who ultimately may acquire the disorder, in order to prevent, cure, delay the onset of, reduce the severity of, or ameliorate one or more symptoms of a disease or recurring disorder. By ‘therapeutic regimen’ is meant the pattern of treatment of an illness, e.g., the pattern of dosing used during therapy.

The therapeutic compositions for use and methods are suitably for the treatment of a subject. As defined herein, the therapeutic uses and methods of treatment or prevention of tissue damage and/or degenerative disease, typically of eye diseases or of any disease or condition associated with such eye diseases may comprise administering the therapeutic compositions or pharmaceuticals as defined herein to a subject or ‘patient in need thereof’. Such a subject or patient is typically an animal, particularly a mammal, more particularly a primate, such as a human or non-human primate. The subject or patient may, for example, be selected from the group comprising, without being restricted thereto, human, primate, monkey, ape, pig, goat, cattle, swine, dog, cat, donkey, or rodents (including mouse, hamster and rabbit). The uses and methods disclosed herein may therefore be put into practice in the field of either human medicine or veterinary medicine.

‘Administering’ or ‘administration’ in the context of the therapeutic uses and methods of treatment typically occurs by administering a ‘therapeutically effective’ amount of the active agent; e.g. of the therapeutic composition or pharmaceutical as defined herein, which is suitable for (intraocular) treatment of eye diseases or disorders as defined herein. Desirably, the therapeutically effective amount is also a ‘safe and effective’ amount of the active agent, As used herein, a ‘safe and effective amount’ means an amount of the active agent or composition as defined herein, that is sufficient to induce a positive modification of an eye disease or disorder as mentioned herein, i.e. to ‘treat’ the disease or disorder (a ‘therapeutically effective’ amount). At the same time, however, a ‘safe and effective amount’ is small enough to avoid serious side-effects and to permit a desirable relationship between advantage and risk. The determination of these limits typically lies within the scope of the sound medical judgment of a skilled practitioner in the art, and may vary in connection with the particular eye disease or disorder to be treated, and also with the age, sex and physical condition (e.g. weight) of the patient to be treated, including the patient's diet, the severity of the condition, the duration of the treatment, the nature of the accompanying therapy, of the particular pharmaceutically acceptable carrier used, and similar factors, which are within the knowledge and experience of the skilled medical practitioner. The amount to be used may also vary according to the time of and/or route of administration. In the context of the present invention the expression ‘safe and effective amount’ may thus mean an amount of the therapeutic or pharmaceutical composition suitable for the (intraocular) treatment of eye diseases or disorders that is suitable to exert one or more beneficial effect of a ‘treatment’.

As defined herein, the therapeutic uses or methods of treatment or prevention of eye diseases or disorders, or of any disease or condition associated with such eye diseases or disorders, may comprise administering the therapeutic or pharmaceutical composition of the invention in combination with at least one additional active or therapeutic agent. The at least one additional active or therapeutic agent may be selected from one or more of a neuroprotective factor, an anti-angiogenic factor and/or any other agent suitable for (intraocular) treatment of eye diseases or disorders or a condition associated with such eye diseases or disorders. Where more than one active agent or therapeutic composition is to be administered ‘in combination’, such combination encompassed administration of the active agents simultaneously, separately, or sequentially, as may be considered appropriate.

Administration sites in the context of the present invention include any appropriate route of administration taking into consideration the disorder, organ or tissue which is to be treated, for example, where the eye is to be treated, intraocular administration, any surface of the eye or the retina, the iris or its tissues or surrounding areas, which may be treated, without being limited thereto, by injection or implantation. Alternatively, therapeutic compositions, e.g. cells according to the disclosure, encoding chemokine receptors for expression, and/or the at least one additional active or therapeutic agent may be implanted into the anterior chamber, the lens capsular bag (e.g., after surgery), the subretinal space, and the suprachoroidal space. The implantation of the cells may be performed by injection or any other surgical or non-surgical procedure to bring the cells into the interior of eye, defined as all volume inside of the sclera. Suitably, the compositions and/or active agents of the disclosure are administered by injection or implantation into the intraocular space. In some suitable embodiments, intraocular deliver is directly to the subretinal space. As used herein, the term ‘subretinal delivery’ refers to the administration to the location in the retina between the photoreceptor cells and the retinal pigment epithelium cells.

Suitable therapeutic compositions and pharmaceuticals of the disclosure may comprise genetically modified cells, particularly stem cells/progenitor/precursor cells. As used herein, a cell is ‘genetically modified’ if either it or any of its precursor cells have had nucleic acid artificially introduced thereinto. Methods for generating genetically modified cells include the use of viral or non-viral gene transfer (e.g. plasmid transfer, phage integrase, transposons, or viral vectors).

A nucleic acid that has been introduced into a cell according to this disclosure may be termed an ‘exogenous’ nucleic acid, polynucleotide, gene or expression construct. An ‘exogenous’ molecule is a molecule that is not normally present in a cell but can be introduced into the cell by one or more genes, biochemical or other methods. It will be understood that ‘normal presence in a cell’ is measured for a particular stage of development and environmental conditions of the cell. For example, molecules that exist only during the development of particular tissue are exogenous molecules if they are not present in a mature/adult tissue. Exogenous molecules may include exogenous nucleic acids and/or exogenous polypeptides; for example, this may include genes that have been introduced into the cell, or proteins that have been caused to be expressed in a cell in which they would not normally be expressed. Exogenous nucleic acids may be integrated or non-integrated in the genetic material of the target/host cell, or relate to stably transduced nucleic acids. An exogenous nucleic acid in the context of the present disclosure may be part of a proviral sequence, where a proviral sequence is all or part of a provirus, being a virus genome that is integrated into the DNA of a host cell.

Non-integrated exogenous nucleic acids can be DNA or RNA-based, for example including plasmids and mRNA. An exogenous nucleic acid can in some embodiments be an RNA, such as an mRNA, which is delivered to target cells such as the stem cells/progenitor/precursor cells as described herein by any suitable method, such that any encoded polynucleotide, gene or expression construct is expressed in those cells. For example, RNA such as mRNA can be formulated for delivery into target cells by encapsulation in nanoparticles such as lipid nanoparticles; liposomes; nanosomes; microparticles, and so on. Exogenous nucleic acids including DNA and RNA can be delivered into target cells without encapsulation (i.e. as naked polynucleotides), for example using electroporation methods. A benefit of using non-integrated exogenous nucleic acids, is that potential adverse effects of genome integration (genomic damage, potential neoplastic transformation, and so on) can be avoided. Transient expression of the desired encoded product can also be achieved, particularly in cases where the exogenous nucleic acid is RNA such as mRNA, as this will eventually be cleared from the cell, which can be beneficial in preventing expression from continuing after it is needed. In the context of the present invention, for example, further expression of a chemokine receptor after the stem cells have integrated into the target tissue can be unwanted and potentially deleterious, so transient expression may be preferred.

For the avoidance of doubt, as used herein, the terms ‘nucleic acid’, ‘polynucleotide’, and ‘oligonucleotide’ are used interchangeably and refer to deoxyribonucleotide or ribonucleotide polymers in linear or cyclic form, and in single or double stranded form. For the purposes of the present disclosure, these terms should not be construed as limitations on the length of the polymer. Synthetic and/or modified nucleic acids are also contemplated herein. The term may include nucleotides modified in bases, sugars and/or phosphate moieties (e.g. phosphorothioate backbones) as well as known analogs of natural nucleotides. In general, analogs of certain nucleotides have the same base pairing specificity as their corresponding natural bases.

Similarly, the terms ‘polypeptide’, ‘peptide’ and ‘protein’ are used interchangeably to refer to polymers of amino acid residues whatever the length or tertiary structure thereof. The term also applies to amino acid polymers in which one or more amino acids are chemical analogs or modified derivatives of the corresponding naturally occurring amino acids.

Cells and Tissues:

In the context of the present disclosure, ‘stem cell’ means an undifferentiated cell having differentiation potency and proliferative capacity (particularly self-renewal competence) maintaining the same differentiation potency even after cell division. The term stem cell encompasses subpopulations such as pluripotent stem cells, multipotent stem cells, unipotent stem cells and the like according to the differentiation potency. Pluripotent stem cell refers to a stem cell capable of being cultured in vitro and having a potency to differentiate into any cell lineage belonging to three germ layers (ectoderm, mesoderm, endoderm). A multipotent stem cell means a stem cell having a potency to differentiate into plural types of tissues or cells, though not all kinds. A unipotent stem cell means a stem cell having a potency to differentiate into a particular tissue or cell.

Progenitor cells are descendants of stem cells that may further differentiate to create specialised cell types. A progenitor cell may be multipotent but is only capable of differentiating into cells that belong to the same tissue or organ. By contrast, a precursor cell is more differentiated than a progenitor or stem cell and is only capable of differentiating into a specific cell type; for example, photoreceptor precursor cells may be rod photoreceptor precursor cells or cone photoreceptor precursor cells.

The present invention is particularly directed to treatment of a diseased and/or damaged retina in a target subject. The retina comprises a number of differentiated cell layers; examples of which include the retinal pigment epithelial layer and neural retinal layer, wherein the neural retinal layer includes the outer limiting membrane, the photoreceptor layer (outer nuclear layer), the outer plexiform layer, the inner nuclear layer, the inner plexiform layer, the ganglion cell layer, the nerve fiber layer and the inner limiting membrane. In addition, in some developmental stages the neural retina layer may contain a layer comprising neural retina progenitor cells, e.g. a neuroblastic layer.

In accordance with the present disclosure, the term ‘retinal progenitor cell’ refers to a progenitor cell capable of differentiating into any mature retinal cells constituting a retinal tissue, including photoreceptor, horizontal cell, bipolar cell, amacrine cell, ganglion cell, retinal pigment epithelial cell and Muller cell (the terms Muller cell and Muller glia are used interchangeably herein). The term ‘neural retinal progenitor cell’ refers to a cell that is destined to be the inner layer of the optic cup. It includes, for example, a progenitor cell capable of differentiating into any mature cell constituting a neural retinal layer (retinal layer containing retinal layer-specific neuron) that does not contain retinal pigment epithelium.

Meanwhile, the terms photoreceptor precursors, horizontal cell precursors, bipolar cell precursors, amacrine cell precursors, ganglion cell precursors, retinal pigment epithelial precursors, and Muller cell precursors refer to precursor cells committed to differentiate into photoreceptors, horizontal cells, bipolar cells, amacrine cells, ganglion cells, retinal pigment epithelial cells, and Muller cells, respectively. Particularly preferred precursor cells are photoreceptor precursor cells, such as rod photoreceptor precursor cells or cone photoreceptor precursor cells.

Genetically modified cells according to the invention may be ‘stem cells’, particularly pluripotent stem cells, such as iPS cells or cells derived therefrom. In some aspects and embodiments, the cells are neural stem cells, or neural progenitor cells. In other beneficial aspects and embodiments, the cells are retinal progenitor cells; particularly photoreceptor precursor cells (e.g. rod photoreceptor precursor cells and/or cone photoreceptor precursor cells), or precursor cells of Muller cells.

The stem cells used in accordance with this disclosure can be obtained from any appropriate animal, such as human or non-human primates (such as monkeys, gorillas, apes and chimpanzees), or from animals such as rats, mice, sheep, cattle, pigs, dogs, cats and horses.

A pluripotent stem cell to be used in accordance with the present invention is suitably a pluripotent stem cell of a primate (e.g., human, monkey), and preferably a human pluripotent stem cell (e.g. a human iPS cell). In some particularly beneficial aspects and embodiments, the cells are mesenchymal stem cells, such as human mesenchymal stem cells.

Methods for obtaining such cells are within the knowledge and skill of the skilled person in the art. For example, a pluripotent stem cell can be induced from fertilised egg, clone embryo, germ stem cell, stem cell in a tissue and the like. Examples of the pluripotent stem cell include embryonic stem cells (ES cell), embryonic germ cell (EG cell), induced pluripotent stem cell (iPS cell) and the like.

An ES cell can be produced, for example, by culturing an inner cell mass on a feeder cell or in a medium containing LIF. The production methods of ES cell are described in, for example, WO 96/22362, WO 02/101057, and the like. Embryonic stem cells are also available from various organisations or are commercially available for purchased.

An iPS cell in the present invention may be a cell induced to have pluripotency by reprogramming a somatic or ‘terminally differentiated’ cell. For example, a cell induced to have pluripotency by reprogramming differentiated somatic cells such as fibroblast, peripheral blood mononuclear cell and the like by the expression of a combination of plural genes selected from the group consisting of reprogramming genes including Oct3/4, Sox2, Klf4, Myc (c-Myc, N-Myc, L-Myc), Glisl, Nanog, Sall4, lin28, Esrrb and the like (e.g., mouse iPS cell: Takahashi & Yamanaka (2006), Cell, 126(4):663-76; human iPS cell: Takahashi et al., (2007), Cell, 131(5):861-72; and somatic cell: Hou et al., (2013), Science, 341:651-654). iPS cells may be beneficial in that they can differentiate to make derivatives of all three germ layers. Thus, the resultant differentiated cells can be from the same germ layer or from different germ layers. Non-limiting examples of differentiated cell products that can result from a single iPS cell source in accordance with the invention includes retinal epithelium, retinal progenitors, neural stem cells, dopaminergic neurons, astrocytes, hepatocytes, endothelial cells and mesenchymal cells. Accordingly, the therapeutic uses and methods disclosed herein may result in repair and/or replacement of neural stem cells, retinal epithelium and retinal progenitor cells. Mesenchymal stem cells may also be produced from an iPS cell line.

Mesenchymal stem cells (MSCs) are multipotent stem cells that can proliferate and differentiate readily into lineages including osteoblasts, myocytes, chondrocytes, and adipocytes (Pittenger, et al., (1999), Science, 284:143; Prockop (1997), Science, 276:71). For example, in vitro studies have demonstrated the capability of MSCs to differentiate into neuronal-like precursors (Woodbury, et al., (2002), J. Neurosci. Res., 69:908), and various other cell types. In recent years, since mesenchymal stem cells have the function of repairing and replacing damaged neurons, they have been used in the development of the treatment of brain trauma, stroke and neurodegenerative diseases (Yoo et al., (2008), Exp. MoL. Med., 40:387-97); accordingly, mesenchymal stem cells (or MSCs) are an attractive cell type for use in the present invention, for the treatment of retinal diseases, disorders and damage.

MSCs may be derived from adult stromal tissues, including but not limited to bone marrow, umbilical cord, amniotic membrane, and amniotic fluid, adipose tissue, pulp cavity, skeletal muscle and skin. MSCs can be collected from various sources by methods known to the person skilled in the art; usually stem cells are isolated from tissues. In particular embodiments, e.g. for use in the treatment or humans, the MSCs are beneficially derived from humans. In other embodiments, MSCs may be derived from mice. Conveniently, such MSCs may be isolated from the skin (particularly skin mesenchymal stem cells), which may provide various advantages of abundant sources, convenient materials, high number and purity of isolated cells, and the advantages of maintaining the characteristics of stem cells after multiple subcultures. One particularly beneficial source of MSCs is bone marrow.

Since MSCs gradually lose their viability as the number of generations increases, MSCs from the 1^{st} to 20^{th} generations are usually used, for example, from the 5^{th} to 18^{th} generations; suitably from the 10^{th} to 16^{th} generations.

In other aspects and embodiments, the cell for use in accordance with the disclosure may be a somatic or terminally-differentiated cell. Suitably, the somatic cell is a cell of the eye, such as a photoreceptor cell, horizontal cell, bipolar cell, amacrine cell, ganglion cell or retinal pigment epithelial cell or Muller cell.

In other aspects and embodiments, the cell for use in accordance with the disclosure may be a hybrid cell, e.g. which has been artificially created by fusing two (or more) different cells. For example, a suitable hybrid cell may be a fusion between a Muller Glia cell and a bone marrow derived stem cell; a fusion between an MSC or bone marrow derived stem cells with a neuron cell or a precursor cell; or a fusion between an iPSC-derived progenitor cell with a Muller Glia cell.

Genetically-Modified Cells:

The construction of the genetically-modified cells described herein may be carried out using techniques known to a person skilled in the art. As described herein, genetically modified cells may express exogenous proteins/polypeptides; particularly chemokine receptors such as CCR5, CXCR6, CCR1 and/or CXCR2. The sequence of the chemokine receptor is suitably selected to be compatible or identical to the species of the cell in which it is used, which is suitably selected to be compatible or identical to the species of the subject or patient into which the cell is to be introduced.

Genetically modified viruses (viral vectors) can be used for the delivery of genes into cells in accordance with various aspects of the invention. Suitable viral vectors may include those derived from retroviruses (such as influenza, simian immunodeficiency virus (SIV), human immunodeficiency virus (HIV), lentivirus, and Moloney murine leukaemia); adenoviruses; adeno-associated viruses (AAV); sendai virus vector (SeV); herpes simplex virus (HSV); and chimeric viruses. Adeno-associated virus (AAV) vectors are considered particularly useful for targeting therapeutic peptides to the central and peripheral nervous systems and to the brain, and have been used to generate vectors for gene transfer in the field of gene therapy and cellular engineering. AAV vectors for use, include without limitation, AAV1, AAV2, AAV3, AAV4, AAV5, AAV6, AAV6.2, AAV7, AAV8, AAV9, rhlO, AAVrh64RI, AAVrh64R2 and rh8 or variants thereof. For example, preferred viral vectors for delivery of exogenous nucleic acids into target cells may be based on the AAV2/1 and AAV2/9 viral subtypes. It is considered that a single viral vector may be engineered to deliver one, or more than one gene into cells in accordance with the invention, which may all be chemokine receptors as described herein, or may include other factors such as paracrine factors. For example, the viral vector may deliver at least two, at least three, at least four, or at least five chemokine receptors as described into the cells.

Lentiviruses are members of retroviridae family of viruses. Lentivirus vectors are generated by deletion of the entire viral sequence with the exception of the LTRs and cis acting packaging signals (M. Scherr et al., (2002), Curr. Gene Ther., 2(1):45-55). One distinguishing feature of these vectors from retroviral vectors is their ability to transduce dividing and non-dividing cells as well as terminally differentiated cells. Lentiviral vectors are particularly considered in instances where more than one gene is to be delivered to a cell, as described above, due to the relatively large packaging capacity of such vectors. Optimised lentiviral vectors can therefore be produced which encode one, or more than one gene for delivery to a cell.

In order to deliver a desired nucleic acid sequence or gene into a target cell using a viral vector it is generally necessary to construct a recombinant viral vector (e.g. a ‘recombinant’ AAV9 (or rAAV9)-derived vector). A ‘recombinant’ viral vector in the context of the invention refers to a virus, such as an adeno-associated virus type 9, that is not naturally occurring and comprises a polynucleotide sequence of interest that is not of that viral origin (i.e. an exogenous or heterologous sequence, such as a transgene to be delivered to a cell) and/or of which the natural DNA or protein(s) (Cap and/or Rep) has been modified, for example to alter its tropism. For example, the rAAV9-derived vector comprises the exogenous polynucleotide sequence of interest under the control of a specific promoter—typically as well as a modified AAV9 VP1 capsid protein, so as to provide efficient delivery and high-level expression of the polynucleotide into the target cell. In accordance with the invention, the target cell may be any stem cell, particularly a mesenchymal stem cell, or a cone or rod photoreceptors precursor cell.

Thus, in some embodiments, the genetically modified cell as described herein is characterised in that the exogenous nucleic acid comprises viral vector sequences, for example, in the form of a viral expression construct. In other embodiments, the genetically modified cell as described herein is characterised in that the exogenous nucleic acid is a non-viral expression construct.

Non-viral methods may also be employed for introducing exogenous nucleic acids into target cells. Such alternative strategies may include conventional plasmid transfer and/or the application of targeted gene integration through the use of nuclease-based gene editing, integrase or transposase technologies. In some cases these techniques have the advantage of being both efficient, and often site-specific in their integration. Physical methods to introduce vectors/plasmids into cells are known to a skilled person. Examples of suitable techniques include electroporation, or the use of liposomes or protein transduction domains, calcium phosphate, lipofection, or RetroNectin. Gene transfer methods may also use direct injection of polynucleotide/vector or the like.

As discussed above, it is also contemplated to use RNA-based approaches, including mRNA, for introducing exogenous nucleic acids into target cells. Such approaches can achieve transient expression, as RNA will eventually be cleared from the cell, which can be beneficial in preventing expression from continuing after it is needed. In the context of the present invention, for example, further expression of a chemokine receptor after the stem cells have integrated into the target tissue can be unwanted and potentially deleterious, such that transient expression may be preferred.

In some embodiments, genetically-modified pluripotent stem cells can be produced by using, for example, a homologous recombination technique. For example, a target gene or other nucleic acid sequences on the chromosome can be modified using the methods described in Manipulating the Mouse Embryo, A Laboratory Manual, Second Edition, Cold Spring Harbor Laboratory Press (1994); Gene Targeting, A Practical Approach, IRL Press at Oxford University Press (1993); Biomanual Series 8, Gene Targeting, Making of Mutant Mouse using ES cell, YODOSHA CO., LTD. (1995); and so on.

Production of a targeting vector used for homologous recombination of a target nucleic acid sequence, and selection of a homologous recombinant can be performed according to the methods described in Gene Targeting, A Practical Approach, IRL Press at Oxford University Press (1993); Biomanual Series 8, Gene Targeting, Making of Mutant Mouse using ES cell, YODOSHA CO., LTD. (1995); and so on. The targeting vector can cause replacement of a gene sequence or insertion or additional polynucleotide sequence, as desired. Various selection methods, such as positive selection, promoter selection, negative selection, polyA selection can be used to identify genetically modified cells.

Genetically-modified cells can, as desired, be cultured and stored by any of the appropriate methods that will be known to the person skilled in the art.

Expression of exogenous proteins is typically achieved by transformation or infection of cells with exogenous polynucleotides comprising one or more gene that encodes the desired protein or polypeptide. As noted above, any given gene delivery method is encompassed by the invention, such as viral or non-viral vectors, as well as biological or chemical methods of transfection, or combinations thereof. Such methods can achieve, as desired, either stable or transient gene expression according to the system used. Similarly, the gene product may be produced constitutively or by inducible expression methods, as known to the person of skill in the art.

In some embodiments, the exogenous gene may integrate into the host cell genome at a desired, targeted site or, in some embodiments, at a random integration site. In other embodiments the exogenous gene may be maintained stably, autonomously and separately from the host cell genome. In some embodiments of the invention it is desirable to infect target cells with a SeV vector containing an exogenous gene or nucleic acid sequence. SeV genetic material does not integrate into the host genome or undergo genetic recombination. Thus, SeV replicates only in the cytoplasm without DNA intermediates or a nuclear phase. In other embodiments of the invention it is desirable to infect target cells with an AAV vector containing an exogenous gene or nucleic acid sequence. AAV genetic material integrates into the host genome at a specific site in the genome.

The invention also encompasses the use of more than one virus, or a virus and other gene editing event or genetic modification, including the use of or mRNA, siRNA, miRNA, or other genetic modification in order to manipulate gene expression within a target cell.

Protein Expression in Cells:

In some embodiments the invention encompasses genetically-modified cells, as described herein, in which an exogenous gene is introduced into the target cell to cause expression of an exogenous protein. In other embodiments, the invention encompasses genetically-modified cells, as described herein, in which an exogenous nucleic acid sequence is introduced into the target cell to cause expression or in some cases overexpression, of an endogenous gene to produce an exogenous polypeptide product (for example, where the endogenous gene is not typically expressed or is expressed at lower than the desired effective level).

In some aspects and embodiments, the genetically-modified cell, as described herein, is characterised in that the promoter or promoter/enhancer combination is operably linked to the exogenous nucleic acid or gene sequence. The term ‘operably linked’, when applied to nucleic acid sequences, for example, in an expression vector or construct, indicates that the sequences are arranged so that they function cooperatively in order to achieve their intended purposes, i.e. a promoter sequence allows for initiation of transcription that proceeds through a linked coding sequence as far as the termination sequence. In some embodiments, the use of a constitutive promoter for expression of the one or more chemokine receptors in accordance with the invention is desirable. In other embodiments, expression of exogenous nucleic acid or gene sequences is inducible, which can be beneficial where constitutive expression of the exogenous polypeptide is not necessary or not desired. In some embodiments, for example, where more than one exogenous nucleic acid or gene sequence is introduced into a target cell, each nucleic acid or gene sequence can be operatively associated with a different promoter or promoter/enhancer combination, such that one gene may be constitutively expressed and another gene may be inducibly expressed. In instances where the exogenous nucleic acid is RNA, the at least one promoter and/or enhancer as discussed herein is also considered to apply to any sequence which allows for the expression of any encoded polypeptide in the RNA, for example, start codon, 3′ or 5′ UTR, ribosomal entry sites, and so on.

As used herein ‘inducible’ expression relates to system of gene expression, in which the gene of interest, such as the chemokine receptor, is preferably not expressed, or in some embodiments expressed at negligible or relatively low levels, except in the presence of one or more (small) molecules (i.e. an inducer) or other defined set of cellular conditions that allows for gene expression. Inducible promoters may be naturally occurring promoter sequences, or synthetic promoters comprising any given inducible element. Inducible promoters may thus encompass those where induction is a result of particular tissue- or micro-environments or combinations of biological signals present in particular tissue- or micro-environments, or to promoters induced by external factors, for example by administration of a small drug molecule or other externally applied signal.

Chemokines and Chemokine Receptors:

Chemokines refer to a sub-group of cytokines (signaling proteins) secreted by cells. Cytokines have the ability to induce directed chemotaxis in nearby responsive cells; they are chemotactic cytokines. Proteins are classified as chemokines according to shared structural characteristics such as small size (typically approximately 8-10 kilodaltons in size), and the presence of four cysteine residues in conserved locations that are key to forming their three-dimensional shape. Chemokines have been classified into four main subfamilies: CXC, CC, CX3C and XC. All of these proteins exert their biological effects by interacting with G protein-linked transmembrane receptors called chemokine receptors, which are selectively found on the surfaces of their target cells.

The major role of chemokines is to act as chemoattractants to induce or direct migration of immune cells. Cells that are attracted by chemokines follow a signal of increasing chemokine concentration towards the source of the chemokine. Some chemokines control cells of the immune system during processes of immune surveillance, some chemokines have roles in development. Other chemokines are inflammatory and are released from a wide variety of cells in response to bacterial infection, viruses and agents that cause physical damage. Their release is often stimulated by pro-inflammatory cytokines such as interleukin 1. Inflammatory chemokines function mainly as chemoattractants for leukocytes, recruiting monocytes, neutrophils and other effector cells from the blood to sites of infection or tissue damage. Certain inflammatory chemokines activate cells to initiate an immune response or promote wound healing.

In accordance with the present disclosure, the inventors identified a problem in the prior art that cells transplanted into the eye do not efficiently migrate and integrate into the retina for purposes of retinal therapy, and this is especially true following intravitreal injection. Similar problems of low migration and integration have been encountered in cell replacement therapies attempted in other tissues. The inventors therefore decided to investigate gene expression levels in healthy and damaged retina, especially in relation to expression of chemokines, with the aim to identify phenotypic differences between healthy and damaged retina. In particular, in relation to damaged retinal tissue, the inventors studied two different models of retinal degeneration: (i) using a pharmacological model of ganglion/amacrine cell degeneration to obtain retinal tissue resulting from chemically-induced damage (NMDA); and (ii) using a genetic model of retinitis pigmentosa (RP). Both mice and human retinal tissue was studied.

As described elsewhere herein, the inventors discovered that various chemokines are expressed differently between healthy and damaged/diseased retina; and some patterns exist in the chemokine expression profiles of damaged retinal tissue induced by different mechanisms of damage. The inventors postulated that expression of chemokine receptors in transplanted cells used for the treatment of eye diseases, and particularly for the treatment of retinal diseases and disorders may improve therapeutic effect, through increased, more efficient migration to and integration into the sites of retinal tissue damage.

The inventors then investigated the relationship between expression of chemokines and chemoattraction based on various different chemokine receptors exogenously expressed in different cell types, to identify patterns of behaviour and potential cell migratory effects both in vitro and in vivo to determine which chemokine receptors, or combinations of chemokine receptors, provide the most effective cell targeting towards potential damaged retinal tissue. Ultimately, it was further investigated which genetically-modified cells were capable of both migrating to relevant sites of retinal tissue damage and then integrating into or otherwise assisting in the repair or regeneration of retinal tissue and cells.

Thus, the genetically-modified cells of the present invention, which express exogenous chemokine receptor proteins may be self-targeting therapeutic vessels, which travel to sites of particular damage, especially of the retina, and then integrate into or otherwise provide a localised therapeutic effect.

It will be appreciated that the exogenous chemokine receptor may be encoded by a gene or cDNA sequence for a chemokine receptor from any suitable species, such as human or non-human primates (such as monkeys, gorillas, apes and chimpanzees), or from animals such as rats, mice, sheep, cattle, pigs, dogs, cats and horses, as in the case of useful cells according to the disclosure. Moreover, it will be appreciated that it may be beneficially, e.g. to reduce host immunogenicity to match the encoding sequence—or still more appropriately, the polypeptide sequence of the encoded chemokine receptor to the host system. Thus, in compositions for use in therapy and therapeutic methods as disclosed herein, the chemokine receptor is suitably from the same species as the subject/recipient. Accordingly, the chemokine receptor encoding sequence and/or polypeptide may particularly be a primate sequence, such as a human or non-human primate (as disclosed herein); more particularly the chemokine receptor is a human chemokine receptor sequence. In other embodiments, it may, for example, be convenient to use a mouse homologue of a human chemokine receptor. Mouse homologues may be particularly useful in in vivo and in vitro assays.

Particularly beneficial chemokine receptors for use in the aspects and embodiments disclosed herein are Ccr5, Cxcr6, Ccr1, Cxcr2 and/or Ccr3. More particularly, human, non-human primate or mouse homologues of such chemokine receptors.

It is also contemplated that the genetically-modified stem cells as described herein may be capable of secreting one or more endogenous proteins or peptides, such as paracrine factors. These proteins or peptides are intended to repair, maintain and/or protect the cells of a subject, for example, retinal cells. These may be selected from the group comprising VEGF, IL6, IL8, GDNF, NT3, NeuroD1 and/or MCP1.

In some embodiments, the present invention provides for the manipulation of the chemokine profile of a target cell, tissue or organ, in order to improve the integration of stem cells according to the principles described elsewhere herein. For example, in some embodiments, methods of introducing nucleic acids as described herein (for example, viral transfection and/or RNA methods) are applied directly to target host cells within a subject, in order to increase the expression of chemokines by those cells. In this way, a chemokine gradient may be set up that stem cells can follow. The chemokines encoded by introduced nucleic acids may be suitable ligands for one or more of the chemokine receptors described herein, for example, for one or more of Ccr5, Cxcr6, Ccr1, Cxcr2 and Ccr3. The chemokines may be one or more of CCL3, CCL5, CCL7, CCL23, CCL4, CCL3L1, CCL11, CCL26, CCL13, IL8, CXCL1, CXCL2, CXCL5, and/or CCL16. As a result, methods to increase chemokine expression in target cells can be used in conjunction with methods of stem cell transplantation. The stem cells used in such methods can be wild-type (that is, substantially not genetically-modified) or may be genetically modified. In embodiments, the stem cells may be genetically modified as described herein, that is, by the introduction of an exogenous nucleic acid encoding a chemokine receptor.

Therapeutic/Pharmaceutical Compositions:

The therapeutic agents of the present invention include genetically-modified cells; particularly genetically-modified iPS cells, MSCs and/or photoreceptor precursor cells; and for therapeutic uses or in methods of treatment are desirably comprised in a therapeutic or pharmaceutical composition.

Such compositions may comprise, in addition to the genetically-modified cells, a pharmaceutically acceptable excipient, carrier, buffer, stabiliser or other materials well known to those skilled in the art. Such materials should be non-toxic and should not interfere with the efficacy of the active ingredient (i.e. the cell of the invention). The precise nature of the carrier or other material may be determined by the skilled person according to the route of administration, e.g. intraocular injection; particularly subretinal injection.

A pharmaceutical composition for use as an injectable is preferably in liquid form, such as a solution or suspension, which preferably contain water (aqueous formulation) or may be emulsified. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. For injection, the active ingredient may desirably be in an aqueous formulation, which is pyrogen-free and has suitable pH, isotonicity and stability. The term ‘aqueous formulation’ is defined as a formulation comprising a significant proportion (for example, at least 50% w/w) of water. Likewise, the term ‘aqueous solution’ is defined as a solution comprising a significant proportion (for example, at least 50% w/w) of water, and the term ‘aqueous suspension’ is defined as a suspension comprising a significant proportion (for example, at least 50% w/w) of water.

Therapies:

Therapeutic compositions and pharmaceuticals of the invention may be delivered to a subject by any appropriate means. Where the eye is to be treated, such delivery may be intraocular, by intravitreal injection or subretinal injection.

The fovea accounts for less than 1% of the retinal surface area in primates yet it provides the input to about 50% of the cells in the primary visual cortex. The high concentration of cones in the fovea, the thinnest and most delicate part of the retina, allows for high acuity vision, and it is of utmost importance to preserve the unique functions and architecture of the cones in this area during therapeutic interventions. Foveal cones can be targeted via different administration routes, using either subretinal or intravitreal injections, but detaching the fovea might lead to mechanical damage, especially in the degenerating retina. Thus, intravitreal injections are beneficial in being a relatively simple way of delivering therapeutics without retinal detachment. Gene therapy vectors can target the outer retina via intravitreal injections in rodents without damage to the photoreceptor. However, safe and efficient gene delivery to primate cones via intravitreal injection has been difficult to achieve, perhaps as a result of dilution of the therapeutic. The use of genetically-modified cells according to the invention that are capable of efficiently and effectively migrating to particular sites of retinal cell damage may therefore allow convenient, safe and efficacious therapeutic delivery, without risk of damaging existing eye tissues, e.g. by enabling effective delivery to the intravitreal space or the subretinal space, as may be desired.

In various aspects and embodiments, the genetically modified cells of the invention may be used in methods comprising administering at least one additional therapeutic agent. Thus, it is envisaged that the therapeutic/pharmaceutical compositions of the invention may be used in a method of combination therapy with another composition of the invention and/or optionally one or more additional therapeutic agent. For example, an additional therapeutic agent may be any agent that provides a beneficial therapeutic effect in the treatment of eye disease or, more specifically, in the treatment of eye disorders associated with damage of the retina. In embodiments, the additional therapeutic agent may be another genetically-modified cell (as described herein), which is genetically modified to express (and secrete) e.g. a neuroprotective factor (a compound or polypeptide which protects neurons from apoptosis or degeneration, e.g. GLP-1 peptides), an anti-angiogenic factor (a compound or polypeptide which inhibits angiogenesis; e.g. endostatin) and/or any other protein or protein-like substance or a fragment or variant thereof suitable for (intraocular) treatment of eye diseases or disorders as defined herein. Alternatively, the additional therapeutic agent may be a naked nucleic acid or viral vector capable of infecting cells within the eye and particularly retinal cells, or may be a pharmaceutical or therapeutic compound in an isolated form, e.g. a drug. However, administration via cells according to the invention may allow the circumvention of problems of repeated administration of such factors. As previously described, any such additional therapeutic agent can be administered separately, simultaneously with, or sequentially with the genetically-modified cell of the invention, for example, by the same or similar routes of administration.

Use of MSCs may be particular beneficial in the therapeutic uses and methods described herein, in view of their paracrine activity, which has been studied and characterized (Ding et al., (2017), Int. J. Mol. Sci., 18(8):1406). Indeed, the plethora of cytokines and neurotrophic factors they secrete may be important for the repair of injured tissues, and may also serve to decelerate disease progression (e.g. see Johnson et al., (2014), Brain, 137(2):503-519; Wilkins et al., (2009), Stem Cell Research, 3(1):63-70). Additionally, MSCs display a broad differentiation potential, such that given the appropriate environmental conditions, MSCs can be converted into a variety of neural cell types, including photoreceptors (Gregory et al., (2005), Experimental Cell Research, 306(2):330-335). Furthermore, MSCs can be easily expanded ex vivo, which provides for abundant starting material for transplantation (Beyer Nardi & da Silva Meirelles (2006), Handbook of Experimental Pharmacology, 174:249-82). Prolonged ex vivo expansion is currently not possible for other cell sources such as hematopoietic stem cells (Ribeiro-Filho et al., (2019), Cells, 8(12):1628).

### EXAMPLES

The invention will now be further illustrated by way of the following non-limiting examples.

Unless otherwise indicated, commercially available reagents and standard techniques in molecular biological and biochemistry were used.

Materials and Methods

Cell and Tissue Culture

Primary MSCs derived from the bone-marrow of mice (C57BL/6) were purchased from GIBCO (S1502-100). They were produced from the bone marrow isolated from C57BL/6 mice at≤8 weeks of gestation through mechanical and enzymatic digestion and were maintained in DMEM/F-12-GlutaMAX supplemented with 10% fetal bovine serum (FBS), penicillin (100 U/ml) and streptomycin (100 μg/ml). For all experiments MSCs were used between passage 10 and 16. Human retinal pigmented epithelial (RPE) cells were purchased from ATCC (CRL-2302) and maintained in DMEM/F-12-GlutaMAX supplemented with 10% fetal bovine serum (FBS), penicillin (100 U/ml) and streptomycin (100 μg/ml). Mouse eyecups were prepared by removing the cornea, the iris and the vitreous and then cultured in serum-free (SF) DMEM/F-12-GlutaMAX with penicillin (100 U/ml) and streptomycin (100 μg/ml). Human retina were dissected and cultured in SF Neurobasal A medium supplemented with GlutaMAX (0.5%), N2 (1×), B27 (1×), penicillin (100 U/ml) and streptomycin (100 μg/ml).

Animal Care and Treatment

Mice were maintained under a 12-hour light/dark cycle with access to food and water ad libitum, in accordance with the Ethical Committee for Animal Experimentation (CEEA) of the Government of Catalonia. The CEEA of the Parc de Recerca Biomèdica de Barcelona (PRBB, Spain) reviewed and approved all animal procedures. Additionally, procedures and experiments were performed in accordance with the ARVO Statement for the Use of Animals in Ophthalmic and Vision Research, and with ARRIVE (Animal Research: Reporting of In Vivo Experiments) guidelines (Kilkenny et al., (2011), J. Cerebral Blood Flow & Metabolism, 31(4):991-3). Male and female C57BL/6 mice between 8-12 weeks were used for the experiments involving transplantation following NMDA-damage; rd10 (and corresponding WT control) C57BL/6 mice were transplanted at post-natal day 18 (P18) and sacrificed at endpoints detailed in each of the experiments. The CAG-DsRed transgenic mouse line was also used and obtained from the Jackson Laboratory (Stock Number=006051) (Liang et al., (2001), Methods Mol. Med., 47:125-39). In all experiments, animals were assigned randomly to the various treatment groups. A minimum of three mice per treatment group were used. General anesthesia was induced when needed with intraperitoneal injection of ketamine (70 mg/kg) and medetomidine (10 mg/kg). Anesthesia was reversed with atipamezole (2 mg/kg). At endpoints, mice were euthanised using CO₂.

Retinal Damage and Cell Transplantation

Mice were anaesthetised and intravitreally injected with 2 μl of either NMDA (20 mmol/μl; Sigma) or PBS, as a control. Briefly, a 30-G needle was used to carefully make a small, punch incision at the upper temporal ora serrata. The 33-gauge needle of a Hamilton's syringe was then inserted into the incision, angled toward the optic nerve, to inject PBS or NMDA into the vitreous. The needle was left in place for a couple of seconds before being retracted to avoid reflux of the injected solutions.

For cell transplantation, MSCs were detached using Accutase (StemPro® Accutase® Cell Dissociation Reagent, Life Technologies™), counted and resuspended in PBS plus chondroitinase ABC (ChABC, 0.1 U/μl) at a concentration of 150,000 cells/μl. Adult mice that had received NMDA-damage were transplanted intravitreally with 2 μl of MSCs (i.e. 300,000 cells), 12h post-injection. P18 rd10 mice were transplanted subretinally with 1 μl of MSCs (i.e. 150,000 cells), as previously described (Liang et al., (2001), Methods Mol. Med., 47:125-39).

Human Retina Culture

The research adhered to the tenets of the Declaration of Helsinki on research involving human subjects. The experimental protocol was approved by the Ethical Committee for Clinical Research of the Centro de Oftalmologia Barraquer. Human donor eyes were obtained from the “Banc d'Ulls per a Tractaments de Ceguesa”. Written informed consent for the removal and use of the eyes for diagnostic and research purposes was obtained from donors and/or relatives. All of the samples were from donors aged 65-90.

Retina were dissected employing a procedure and a set-up optimised in our laboratory in collaboration with the Centro de Oftalmologia Barraquer. Briefly, the cornea, iris, crystalline and vitreal excess were removed. The retina was then separated from the RPE and from the rest of the eye. After the removal of the periphery and of the vitreal leftovers, the central part of the retina was cultured for 12 h and then processed for experiments.

RNA Extraction and Quantitative Real-Time PCR (qRT-PCR)

RNA was extracted and purified using the RNA Isolation Mini kit (QIAGEN), according to the manufacturer's protocol. Total RNA was treated with DNAsel (QIAGEN) to prevent DNA contamination. The cDNA was produced with SuperScript Ill Reverse Transcriptase Kits (Invitrogen). Quantitative real-time PCR (qRT-PCR) reactions were prepared using Platinum SYBR green qPCix-UDG (Invitrogen) and run in a LyghtCycler 480 (Roche) machine, according to the manufacturer recommendations. qRT-PCR analysis was performed in technical duplicates, for a minimum of three biological replicates (with the exception of human retina with RP, where n=2). qRT-PCR data was normalised to GAPDH expression. The oligos used are listed in Table 1.

For the investigation of NMDA-damage, eye samples were collected 24 hours (24 hpi), 48 hours (48 hpi), 4 days (4 dpi), 7 days (7 dpi) or 4 weeks (4 wpi) post-injection. Rd10 mice were sacrificed at P14, P18, P22 and at 6 months of age (adults).

To study gene expression in human retina, RNA was extracted following 24 hours culturing in SF medium with or without NMDA (1 mM).

TABLE 1

| Gene | Primer FW | Primer RV |
|---|---|---|
| mll1-6 | gccaccttttgacagtgatgaga (SEQ ID NO: 1) | ggacagcccaggtcaaaggt (SEQ ID NO: 2) |
| mCcl5 | tgcagaggactctgagacagc (SEQ ID NO: 3) | gagtggtgtccgagccata (SEQ ID NO: 4) |
| mCcl6 | tctttatccttgtggctgtcc (SEQ ID NO: 5) | tggagggttatagcgacgat (SEQ ID NO: 6) |
| mCcl12 | ggtattggctggaccagatg (SEQ ID NO: 7) | gggacactggctgcttgt (SEQ ID NO: 8) |
| mCxcl1 | agactccagccacactccaa (SEQ ID NO: 9) | tgacagcgcagctcattg (SEQ ID NO: 10) |
| mCxcl2 | actccagactccagccacac (SEQ ID NO: 11) | cagttcactggccacaacag (SEQ ID NO: 12) |
| mCxcl5 | tcttgggtgtgttaagagtgttct (SEQ ID NO: 13) | cacagcagctttctaaaaccataa (SEQ ID NO: 14) |
| mCxcl9 | ccatgaagtccgctgttctt (SEQ ID NO: 15) | tgagggatttgtagtggatcg (SEQ ID NO: 16) |
| mCxcl10 | atcagcaccatgaacccaag (SEQ ID NO: 17) | ttccctatggccctcattct (SEQ ID NO: 18) |
| mCxcl11 | gcggctgctgagatgaac (SEQ ID NO: 19) | cgcccctgtttgaacataag (SEQ ID NO: 20) |
| mCxcl16 | tgaactagtggactgctttgagc (SEQ ID NO: 21) | gcaaatgtttttggtggtga (SEQ ID NO: 22) |
| hIL1-β | tacctgtcctgcgtgttgaa (SEQ ID NO: 23) | tctttgggtaatttttgggatct (SEQ ID NO: 24) |
| hTNF-α | cagcctcttctccttcctgat (SEQ ID NO: 25) | gccagagggctgattagaga (SEQ ID NO: 26) |
| hCCL2 | agtctctgccgcccttct (SEQ ID NO: 27) | gtgactggggcattgattg (SEQ ID NO: 28) |
| hCCL5 | cgctgtcatcctcattgcta (SEQ ID NO: 29) | ggtgtggtgtccgaggaata (SEQ ID NO: 30) |
| hCCL21 | tccatcccagctatcctgtt (SEQ ID NO: 31) | agctcctttgggtctgcac (SEQ ID NO: 32) |
| hCCL22 | cgtggtgaaacacttctactgg (SEQ ID NO: 33) | ccttatccctgaaggttagcaa (SEQ ID NO: 34) |
| hCCL23 | ccaggaggatgaaggtctcc (SEQ ID NO: 35) | catcatgaactctgtctctgcat (SEQ ID NO: 36) |
| hCXCL1 | tcctgcatcccccatagtta (SEQ ID NO: 37) | cttcaggaacagccaccagt (SEQ ID NO: 38) |
| hCXCL2 | cccatggttaagaaaatcatcg (SEQ ID NO: 39) | cttcaggaacagccaccaat (SEQ ID NO: 40) |
| hCXCL3 | aaatcatcgaaaagatactgaacaag (SEQ ID NO: | ggtaagggcagggaccac (SEQ ID NO: 42) |
| | 41) | |
| hCXCL6 | gtccttcgggctccttgt (SEQ ID NO: 43) | cagcacagcagagacaggac (SEQ ID NO: 44) |
| hCXCL9 | tgttcccctttgcttcattc (SEQ ID NO: 45) | gaaaggcactgcattgtgg (SEQ ID NO: 46) |
| hCXCL10 | gaaagcagttagcaaggaaaggt (SEQ ID NO: 47) | gacatatactccatgtagggaagtga (SEQ ID |
| | | NO: 48) |
| hCXCL11 | agtgtgaagggcatggcta (SEQ ID NO: 49) | tcttttgaacatggggaagc (SEQ ID NO: 50) |
| hCXCL16 | gccctttcctatgtgctgtg (SEQ ID NO: 51) | caggtatataatgaaccggcagat (SEQ ID NO: |
| | | 52) |
| mCcr1 | ctgtgtggacaaaatactctgga (SEQ ID NO: 53) | tggggtaggcttctgtgaaa (SEQ ID NO: 54) |
| mCcr3 | gaatcaaagagctggggtca (SEQ ID NO: 55) | caggaggccgatgatgaa (SEQ ID NO: 56) |
| mCcr5 | caactttggggtgataacaagtg (SEQ ID NO: 57) | tggtaaagattatttctgggagaga (SEQ ID NO: |
| | | 58) |
| mCxcr2 | caggaccaggaatgggagta (SEQ ID NO: 59) | tcccctccaaatatccccta (SEQ ID NO: 60) |
| mCxcr3 | gtggccaagtcagtcacctc (SEQ ID NO: 61) | cccacaaaggcatagagcag (SEQ ID NO: 62) |
| mCxcr6 | ccagctttaagtatgccatcg (SEQ ID NO: 63) | ttaaggcaagcccgaaagta (SEQ ID NO: 64) |
| mCyclinD1 | gagattgtgccatccatgc (SEQ ID NO: 65) | ctcctccttcgcacttctgct (SEQ ID NO: 66) |
| mBDNF | cagtggacatgtccggtgggacggtc (SEQ ID NO: | ttcttggcaacggcaacaaaccacaac (SEQ ID |
| | 67) | NO: 68) |
| mNGF | tatactggccgcagtgaggt (SEQ ID NO: 69) | ggacattgctatctgtgtacgg (SEQ ID NO: 70) |
| mPDGFa | gatgaggacctgggcttg (SEQ ID NO: 71) | gatcaactcccggggtatct (SEQ ID NO: 72) |
| mCTNF | gacctgactgctcttatggaatct (SEQ ID NO: 73) | gcctggaggttctcttgga (SEQ ID NO: 74) |

Primers used for qRT-PCR analysis; ‘m’ = mouse, ‘h’ = human sequences.

Chemotactic Assays

Chemotactic assays were performed using transwell inserts (pore size, 8 μm, BD Biosciences—353182) and 12-well culture plates. To test migration towards defined chemokine gradients, lower chambers were loaded with 1.2 ml of SF DMEM/F-12-GlutaMAX medium with either mCcl5, mCxcl1, mCxcl10, mCxcl16 or a combination of Ccl5 and mCxcl16 (all 50 ng/ml, Peprotech). For tests of NMDA-damage, mice were sacrificed 24 hpi; rd10 mice were sacrificed at P18. Human retina were dissected and cultured for an initial 12 hours in Neurobasal A medium, as previously described. Afterwards, both mouse and human retina were cultured for 24 hours in SF DMEM/F-12-GlutaMAX and SF Neurobasal A (with or without 1 mM NMDA) respectively. 1.2 ml of the resulting conditioned medium were loaded in the lower chambers of the transwell. The upper chamber was loaded with 2×10⁵ MSCs in SF medium. The medium used to resuspend MSCs was matched to the medium in the bottom chamber: either DMEM/F-12-GlutaMAX (to test migration towards medium conditioned by mouse retina) or Neurobasal A (to test migration towards medium conditioned by human retina). To test chemokine receptor inhibition, MSCs were incubated for 20 min at 4° C. with small molecule receptors antagonists (used as indicated in Table 2), prior to being used in chemotactic assays.

Transwell plates were incubated for 1.5 hours at 37° C. Afterwards, cells remaining on the upper surface of the inserts were removed with a cotton swab. Tranwells were then washed (PBS), fixed (4% paraformaldehyde—PFA, 10 min) and stained with 5 mg/ml 6-diamidino-2-phenylindole (DAPI, Sigma). For each insert, seven fields were imaged and analysed. Cells were automatically counted using a custom-made macro for the ImageJ software (US National Institutes of Health, Bethesda, Md., USA; http://rsb.info.nih.gov/ij/).

Chemokine Antibody Arrays

Proteome Profiler™ Mouse Chemokine Antibody Array (R&D Systems) was used to assay retinal lysates derived from PBS/NMDA-injected (24 hpi) and from WT/rd10 (P18) mice. Manufacturer's recommendations were followed. Briefly, retina were excised and homogenised in PBS with protease inhibitors (10 μg/ml aprotinin, 10 μg/ml leupeptin and 10 μg/ml pepstatin). After homogenisation, Triton X-100 was added to the sample to a final concentration of 1%. Samples were then frozen at −20° C., thawed and centrifuged at 10,000×g for 5 minutes. Arrays were probed with a total of 200 μg of protein. Membranes were developed by standard chemiluminescence techniques. Pixel intensity was quantified using the ImageJ software. The net level of each protein was calculated by the mean of the individual spot intensity minus the mean of the background intensity. Relative spot intensities are presented as mean±SD.

TABLE 2
Name, catalogue number and working
concentration of selective receptor antagonists

| Compound | Catalogue Number | Receptor | Working |
|---|---|---|---|
| Name | (R&D Systems) | Inhibited | Concentration |
| J 113863 | 2595/10 | Ccr1 | 6 μM |
| SB 328437 | 3650/10 | Ccr3 | 25 μM |
| Maraviroc | 3756/10 | Ccr5 | 7 μM |
| SB 332235 | 5671/10 | Cxcr2 | 8 μM |
| ML 339 | 5943/10 | Cxcr6 | 18 μM |

Lentiviral Constructs and MSC Infection

Mouse Ccr1, Ccr3, Ccr5, Cxcr2, Cxcr3 and Cxcr6 coding sequences (CDSs) were amplified by reverse transcribing total mouse spleen RNA (Superscript Ill RT Kit, Invitrogen) and then amplifying the full-length CDSs by PCR (using the Phusion hot start high fidelity polymerase, Thermofisher). The oligos used are listed in Table 3. Resultant cDNA was C-terminally tagged with an HA and inserted into a lentiviral vector with a p1494 backbone, containing an EF1α promoter. An eGFP reporter was also present, with its expression being driven by a constitutive SV40 promoter (EF1a HA-Receptor-SV40_eGFP). To generate the Ccr5-Cxcr6, double expressing line of MSCs, the constitutive eGFP reporter of the EF1a_HA-Receptor-SV40_eGFP construct was replaced by a hygromycin resistance marker (EF1α_HA-Cxcr6-SV40_Hygro).

For infection, lentiviral particles were produced following the RNA interference Consortium (TRC) instructions for lentiviral particle production and infection in 10 cm plates (http://www.broadinstitute.org/rnai/public/). At day 0 HEK293 cells were plated at a density of 5×10⁴ cells/cm² in p150 plates. At day 1, using the calcium phosphate transfection kit (Clontech 631312), cells were co-transfected with: (A) 19.5 μg pCMV-DR8.2; (B) 10.5 μg pCMV-VSV-G; (C) 30 μg of the EF1α_HA-Receptor-SV40_eGFP or the EF1α_HA-Ccr5-SV40_eGFP+EF1α_HA-Cxcr6-SV40_Hygro construct(s). At day 2, the medium of the transfected HEK293 was replaced with fresh DMEM/F-12-GlutaMAX supplemented with 30% FBS. Meanwhile, MSCs were plated at a density of 5×10⁴ cells/cm². The lentiviral particles-containing medium was harvested from HEK293T cells at 48 hours and 72 hours post-transfection (day 3 and 4), filtered, and directly used for cell infection.

MSCs infected with EF1α_HA-Receptor-SV40_eGFP constructs were FACS-sorted based on fluorescent intensity. Cells transduced with EF1α_Ccr5-SV40_eGFP+EF1α_HA-Cxcr6-SV40_Hygro were FACS-sorted based on fluorescent intensity and subjected to hygromycin selection (50 μg/ml) starting two days after the second round of infection.

TABLE 3
List of primers used to amplify receptors'
CDSs from total mouse spleen cDNA.

| Gene | Primer FW | Primer RV |
|---|---|---|
| mCcr1-CDS | atggagatttcagatttcacagaag | tcagaagccagcagagagc |
| | (SEQ ID NO: 75) | (SEQ ID NO: 76) |
| mCcr3-CDS | atggcattcaacacagatgaaatc | ctaaaacaccacagagatttcttg |
| | (SEQ ID NO: 77) | (SEQ ID NO: 78) |
| mCcr5-CDS | atggattttcaagggtcagttcc | tcataaaccagtagaaacttcatg |
| | (SEQ ID NO: 79) | (SEQ ID NO: 80) |
| mCxcr2-CDS | atgggagaattcaaggtggataag | ttagagggtagtagaggtgtttg |
| | (SEQ ID NO: 81) | (SEQ ID NO: 82) |
| mCxcr3-CDS | atgtaccttgaggttagtgaacgt | ttacaagcccaggtaggagg |
| | (SEQ ID NO: 83) | (SEQ ID NO: 84) |
| mCxcr6-CDS | atggatgatgggcatcaagagtc | ctacaattggaacatactggtggtc |
| | (SEQ ID NO: 85) | (SEQ ID NO: 86) |

Immunofluorescence of Cultured Cells

MSCs were plated into Lab-Tek chambers. The following day, they were washed (PBS), fixed (4% PFA, 10 min) and permeabilised (0.2% Triton X-100 in PBS, 10 min). Non-specific binding of antibodies was blocked by a 1 hour-long incubation with a solution of PBS with 3% BSA, 300 μM glycine and 0.03% Triton X-100. Incubation with primary antibodies lasted 3 hours (at room temperature). Cells were then washed with PBS and incubated with secondary antibodies (1.5 hours, at room temperature). DAPI (5 mg/ml) was used to stain nuclei. Images were acquired using the Leica SP8 confocal microscope. The following antibodies were used: chicken anti-GFP (1:200; ab13970, Abcam); mouse anti-HA (1:150; 11583816001, Roche); anti-chicken Alexa Fluor 488; anti-mouse Alexa Fluor 568. All secondary antibodies were provided by Molecular Probes (Invitrogen) and used 1:1,000 in PBS.

Profiling of Secreted Factors

Proteome Profiler™ Mouse Angiogenesis Array (R&D Systems) was used to assay serum-free (SF) DMEM/F-12-GlutaMAX medium that had been conditioned by either GFP-MSCs or dOE-MSCs, cultured for 24 hours. Arrays were probed with a total of 800 μg of protein. Membranes were developed by standard chemiluminescence techniques. Pixel intensity was quantified using the ImageJ software. The net level of each protein was calculated by the mean of the individual spot intensity minus the mean of the background intensity. Relative spot intensities are presented as mean±SD.

Flow Cytometry Analysis of MSCs and Retinal Samples

For flow cytometry, cultured MSCs were detached with Accutase and collected by centrifugation at 300 relative centrifugal field (rcf) for 5 min. They were resuspended at a concentration of 1×10⁶ cells/ml and incubated with purified rat anti-mouse CD16/CD32 (Mouse BD Fc Block™; BD Pharmingen™) at a concentration of 5 μg/ml (in PBS), 20 min at 4° C., to block non-specific binding of antibodies. Following two washes in PBS, cells were incubated with conjugated primary antibodies (in PBS) for 30 min at 4° C., in the dark. Finally, they were washed (PBS) and resuspended in PBS for flow cytometry. DAPI (5 mg/ml) was added to exclude dead cells. The following antibodies were used: PE anti-mouse CCR1 (FAB5986P; R&D Systems); APC anti human/mouse/rat CCR5 (FAB1802A; R&D Systems); Per-CP anti-mouse CXCR2/IL8 RB (FAB2164C; R&D Systems); Alexa Fluor®700 anti-mouse CXCR3 (FAB1685N; R&D Systems); Alexa Fluor®700 anti-mouse CXCR6 (FAB2145N; R&D Systems); PE anti-mouse CD90.2 (12-0902; eBioscience); PE anti-mouse CD44 (12-0441-82; eBioscience); PE anti-mouse CD34 (551387; BD Biosciences); PE anti-mouse CD45 (553081; BD Biosciences); PE-Cy7 anti-mouse Ly-6A/E (Sca-1) (25-5981; eBioscience); PE-Cy7 anti-mouse CD11b (552850; BD Biosciences). All antibodies were used at a concentration of 10 μl/10⁶cells.

For flow cytometry analysis of retinal samples, retina were dissected from the enucleated eyes and incubated (30 min, 37° C.) in trypsin supplemented with 0.1 mg/ml DNAsel for 20-30 minutes at 37° C. Samples were then mechanically triturated, filtered, pelleted, and re-suspended in PBS. DAPI (5 mg/ml) was added to exclude dead cells. Both NMDA-damaged rd10 eyes were analyzed 4 dpi. All data were processed and analysed with FlowJo (v10).

Fixing, Sectioning and Immunofluorescence

Eyes were enucleated and fixed by immersion in 4% PFA overnight at 4° C.; they were embedded in paraffin the following day. 5 μm thick sections oriented orthogonal to the retinal layers were prepared and processed for either immunofluorescence or haematoxylin/eosin staining. Briefly, for the immunofluorescence, sections were de-paraffinised by sequential treatment with Xilene and EtOH gradient; slices were then placed in a plastic rack with a permeabilisation buffer containing 0.3% Triton X-100 and 0.1 M sodium citrate in PBS (1 hour at room temperature). Antigen retrieval was then performed by boiling the slides for 4 minutes in a domestic microwave. After a wash with cold water, non-specific binding of antibodies was blocked by a 1 hour-long incubation with a solution of PBS with 3% BSA, 300 μM glycine and 0.03% Triton X-100. Sections were then incubated with primary antibodies diluted in PBS, 1.5% BSA (2 overnights at 4° C.). On the following day, slides were washed with PBS and incubated with secondary antibodies for 2 hours at room temperature.

For retinal flat mount immunostaining, whole retina were dissected from previously fixed eye globes, and left an additional 30 min in 4% PFA. They were then permeabilised (0.3% Triton X-100 in PBS, 1.5 hour at room temperature). Non-specific binding of antibodies was blocked by a 1 hour-long incubation with a solution of PBS with 3% BSA, 300 μM glycine and 0.03% Triton X-100. Incubation with primary antibodies lasted 48 hours at 4° C. Retina were then washed with PBS and incubated with secondary antibodies (24 hours at 4° C.). DAPI (5 mg/ml) was also used to stain for cell nuclei.

The following primary antibodies were used: chicken anti-GFP (1:200; ab13970, Abcam); mouse anti-BIII-tubulin (1:200; ab7751, Abcam); mouse anti-smi-32 (1:200; 5598440001, Merk); mouse anti-calbindin (1:200; C7354, Sigma); mouse anti-GFAP (1:200; MAB360, Millipore); rabbit anti-neun (1:200; ab177487, Abcam); mouse anti-rhodopsin (1:200; MAB5356, Millipore); rabbit anti-recoverin (1:200; AB5585, Merck Life Science); rabbit anti-phospho-Histone-H3 (ser10) (1:200; 06-570; Millipore); rat anti-CD90.2 (1:150; 14-0902-82, eBioscience). The following secondary antibodies were used: anti-chicken Alexa Fluor 488; anti-mouse Alexa Fluor 568; anti-mouse Alexa Fluor 647; anti-rabbit Alexa Fluor 568; anti-rat Alexa Fluor 568. All secondary antibodies were used 1:1,000 in PBS. DAPI (5 mg/ml) was used to stain for cell nuclei.

Both retinal flat mounts and sections were mounted with Vectashield (Vector Laboratories, 42 Burlingame, CA, USA) and imaged using either Leica laser SP5 or SP8 confocal microscopy systems.

Images from both sections and whole retinal flat mounts were processed with the ImageJ software. Cell counts were based on analysis of at least three animals. For each section and flat mount, a minimum of three fields were imaged.

Apoptotic Assay

For the apoptotic assays, GFP-MSCs were seeded at a density of 7.5×10³ cells/cm². After 24 hours of culturing, cells were treated with 5 μM doxorubicin for an additional 24 hours to induce apoptosis. The apoptotic bodies released in the medium were collected by centrifugation at 300 rcf for 7 min. Cells were then resuspended in fresh DMEM/F-12-GlutaMAX medium and seeded on top of human retinal pigmented epithelial (RPE) cells, seeded at a density of 2×10⁴ cells/cm² 24 hours prior to the assay. RPE cells and GFP-MSC-derived apoptotic bodies were co-cultured for 1, 3, 6, 12, 24, 48 or 72 hours. At the moment of the analysis, cells were trypsinised, collected and resuspended in PBS. Flow cytometry analysis was performed with a Fortessa Analyzer (BD Biosciences). All flow cytometry data were processed and analysed with FlowJo (v10).

Statistical Analysis

As specified in the figure legends, data is presented either mean±SD. All statistical tests and graphs were generated using the Prism 8.0 software (GraphPad, San Diego, CA). Depending on the experimental setup, we used Mann-Whitney test, Two-tailed Student's T-test or One-Way Anova. In all cases, a p value <0.05 was considered significant (*, P<0.05; **, P<0.01; ***, P<0.001; ****, P<0.0001; ns, not significant). In experiments where internal reference samples were used to normalise data across different replicates, their expression was set to 1. To show the internal variability of these reference samples, SD was calculated relative to the internal average value.

Hybrid/Fused Cells

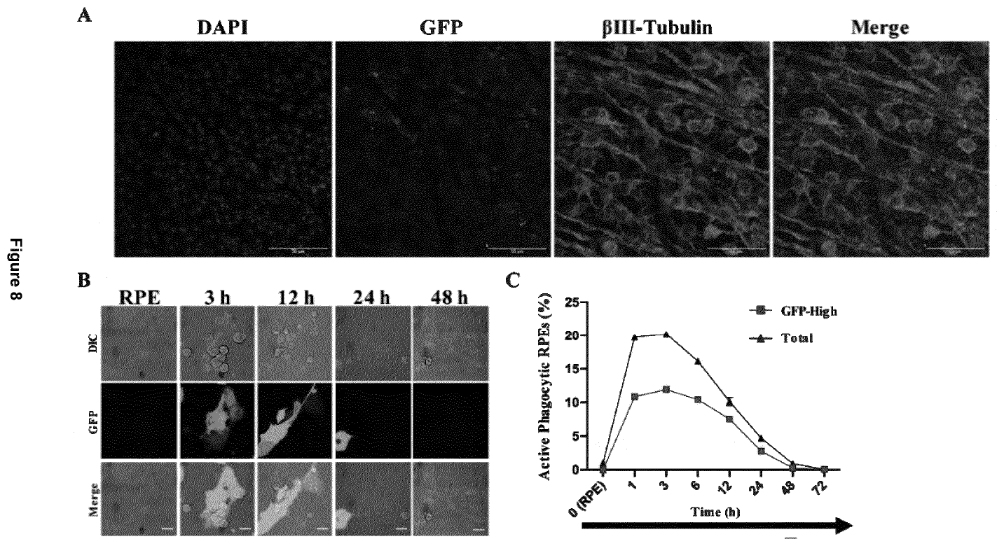

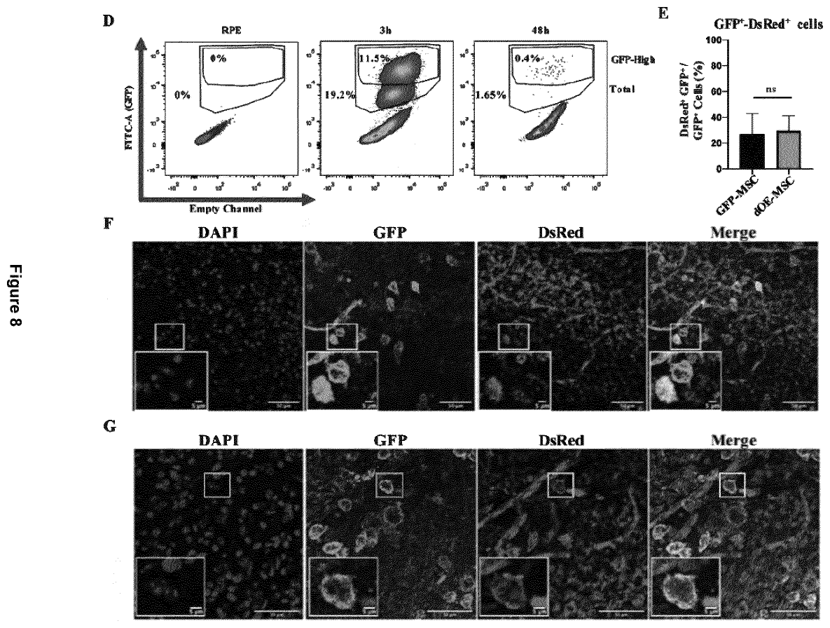

Hybrid or fusion cells for use in accordance with this disclosure can be induced by co-culturing two different cell types which spontaneously fuse together to generate first heterokaryons (i.e. having one cytoplasm and 2 separated nuclei), which then convert in synkaryons (i.e. 1 cytoplasm with 1 nucleus with the double DNA content 4N). The skilled person is well aware from the published literature how to generate desirable hybrid cells by fusion of two different cells in vitro (see e.g. Frade et al., (2019), Science Advances, 5(10), 16 Oct. 2019; Lluis et al., (2010), Stem Cells, 28(11):1940-9). Alternatively, cell fusion can occur in vivo—e.g. in mice after transplanting bone marrow derived stem cells, or after transplanting MSCs in mice, as demonstrated in FIG. 8G (see also Pesaresi et al., (2018), eBioMedicine, 30:38-51; Sanges et al., (2016), J. Clin. Investig., 126(8):3104-3116).

A particular hybrid cell of interest in the context of this disclosure is a hybrid (produced in vitro) between a Muller Glia cell and an MSC. Such a fused hybrid cell is pluripotent and may then be transplanted into a recipient subject as described herein.

### Example 1

Damage-Dependent Soluble Factors Chemoattract MSCs:

Tissue injury induces the release of chemotactic factors (Zlotnik et al., (2006), Genome Biol., 7(12):243). For this reason, the inventors hypothesised the peak of the acute injury response to be concomitant with the maximum secretion of soluble molecules able to chemoattract MSCs. In order to identify such a peak, the expression of Interleukin1-β(II1-β) was analysed at multiple time points following NMDA injection (FIG. 1A), and at various post-natal days in rd10 mice (FIG. 1B). In accordance with previously published studies (e.g. see Chang et al., (2007), Vision Research, 47(5):624-33), we found that II1-β expression peaked 24 hours post-NMDA-injection, and at P18 in the rd10 mouse, eventually reaching low levels one month post-NMDA-damage, and at six months of age (adult) for the rd10 mice.

Transwell-based chemotactic assays were then performed using mouse eyecups, 24-hours post NMDA injection and at P18 for the rd10 mouse (FIG. 1C). We found that media conditioned by degenerating retina stimulated migration of MSCs to a greater extent than media from control samples (see FIGS. 1D, 1E; and FIGS. 2A, 2B). Given the high structural and functional similarity that exists among injury-dependent molecules in mouse and human (Zlotnik & Yoshie (2012), Immunity, 36(5):705-16), we also decided to perform chemotactic experiments using conditioned media from cultured human retina (FIG. 1G). It was found that MSCs migrated more efficiently both towards NMDA-damaged retina and towards retina from patients with RP (FIG. 1F; and FIG. 2C).

In conclusion, the results indicate that upon retinal damage soluble factors are secreted and they can chemoattract MSCs.

### Example 2

Chemotactic Pathways that Elicit Migration of MSCs

We next profiled the inflammatory chemokines present in murine retinal lysates, prepared at 24 hours post-NMDA injection and at P18 for the rd10 mouse. Experiments focused on the two largest and most extensively characterised families of chemokines, i.e. the CC and the CXC. Compared to their control counterparts, degenerating retina showed heightened levels of multiple inflammatory chemokines (FIGS. 2D, 1E). Most notably, levels of Ccl6, Ccl12, and Cxcl16 were increased in both models. In contrast, Cxcl5 was increased only in NMDA-damaged retina, and Ccl5, Cxcl9 and Cxcl10 were increased only in the rd10 retina. Results from the cytokine arrays were validated by gene expression analysis, performed at multiple time points following NMDA injections and postnatal days in the rd10 mice (FIGS. 3A and 3D). It was found that chemokine expression followed a trend that was similar to that of II1-β (FIGS. 3B, 3C, 3E, 3F). A strong, damage-dependent upregulation of Ccl5, Ccl6, Ccl12, Cxcl9, Cxcl10 and Cxcl16, 24 hours post-NMDA injection and at P18 for the rd10 mouse (FIGS. 2F and 2G) was confirmed.

Gene expression changes were also investigated in human retina isolated from deceased donors and cultured ex vivo (FIGS. 3G, 3H). In these studies injury responses were detected both in retina exposed to NMDA and in retina from patients with RP, as indicated by the upregulation of IL1-β and TNF-α (FIG. 3G). Upregulation of IL1-β and TNF-α was accompanied by increased expression of multiple CC and CXC chemokines, including CCL5, CCL22, CCL23 (mCc/6), CXCL3 (mCxcl1), CXCL10, CXCL11 and CXCL16 (FIG. 3H).

Based on the expression data from degenerating retina, various CC and CXC ligand-receptor axes were identified that could potentially be involved in the recruitment of migratory cells. These included Ccl5/Ccl6/Ccl12-Ccr1/Ccr3/Ccr5; Cxcl9/Cxcl10-Cxcr3; and Cxcl16-Cxcr6 (FIG. 2H). The Cxcl1/Cxcl2/Cxcl5-Cxcr2 axis was then further investigated, as it has been shown to recruit MSCs in other degenerative contexts (e.g. Alexeev et al., (2016), Stem Cell Research & Therapy, 7(1):124). Therefore, transwell assays in the presence of small molecule antagonists of chemokine receptors were performed (FIGS. 2I, 2J; and FIG. 3I). In particular, inhibitors of the Ccr1, Ccr3 and Ccr5 receptors were used, along with a Cxcr2 inhibitor and a Cxcr6 inhibitor. Of note, combined inhibition of the three Ccrs receptors was chosen as all three can be bound by Ccl5. In the context of both NMDA- (FIG. 2I) and RP- (FIG. 2J) induced degenerations, we found that migration of MSCs could be significantly reduced either by inhibition of the Cxcr6 receptor, or by combined inhibition of Ccr1, Ccr3 and Ccr5. However, the chemotactic response of MSCs was not affected by the inhibition of Cxcr2. The combined inhibition of Ccrs and Cxcrs receptors led to an even stronger reduction in MSC migration.

In conclusion, the results of these experiments indicate that Ccr1, Ccr3, Ccr5 and Cxcr6 activation can elicit migration of MSCs in the context of retina degeneration.

### Example 3

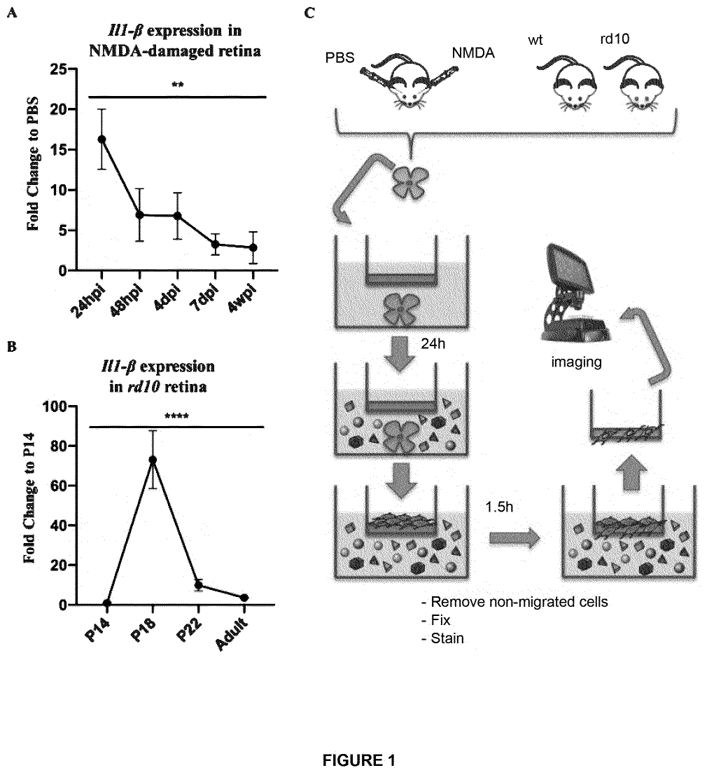

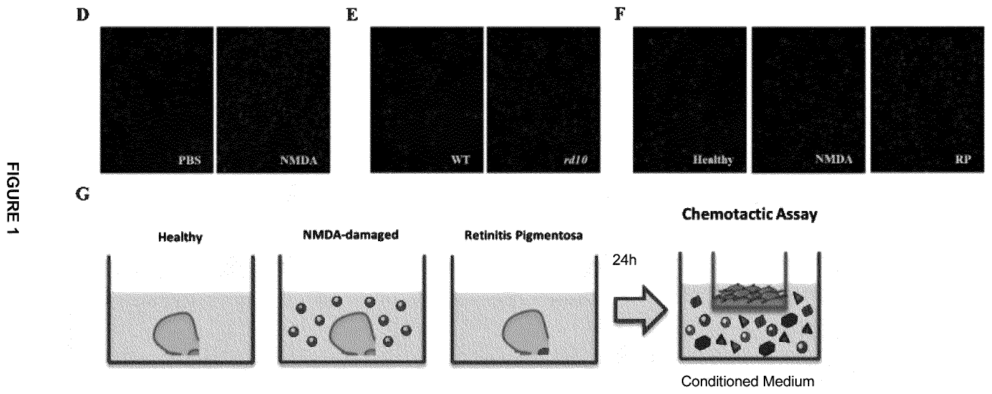

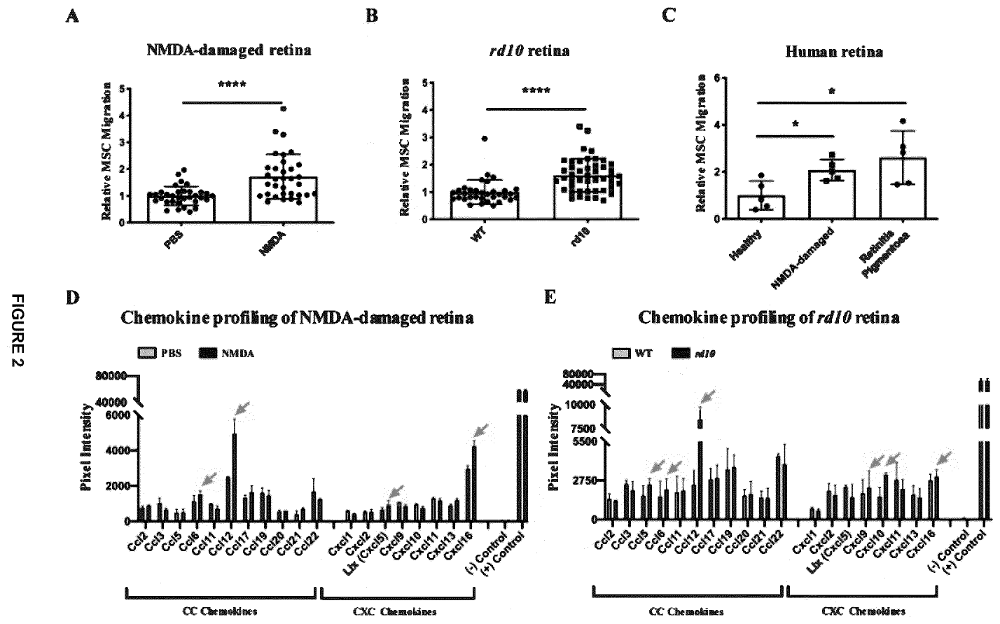

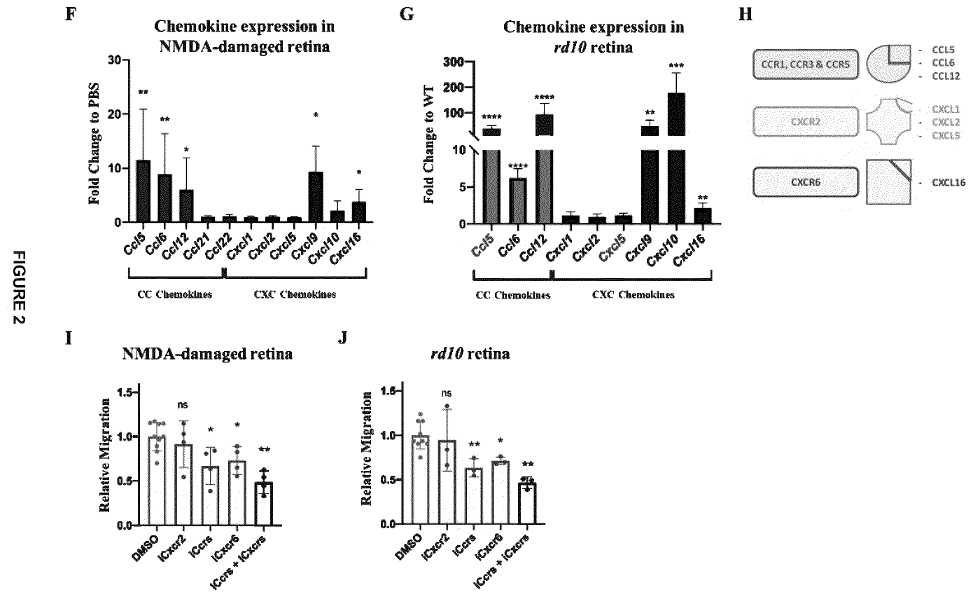

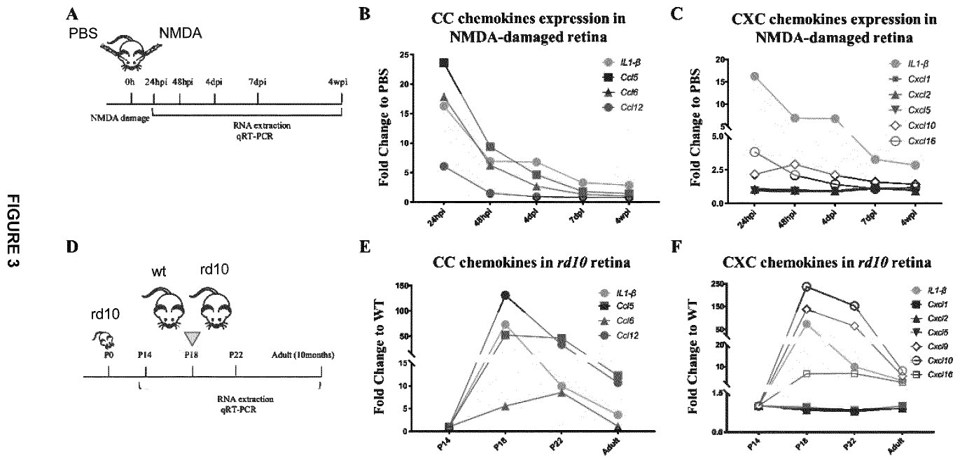

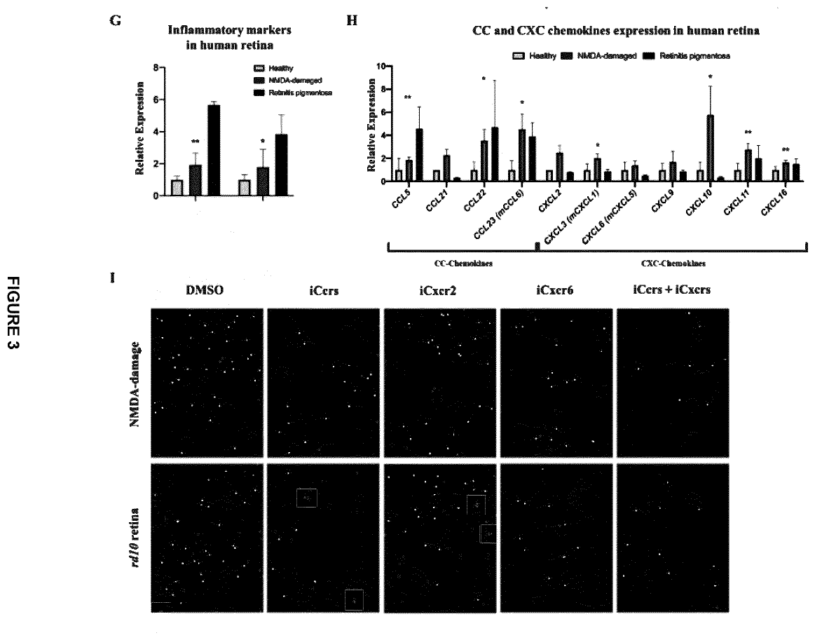

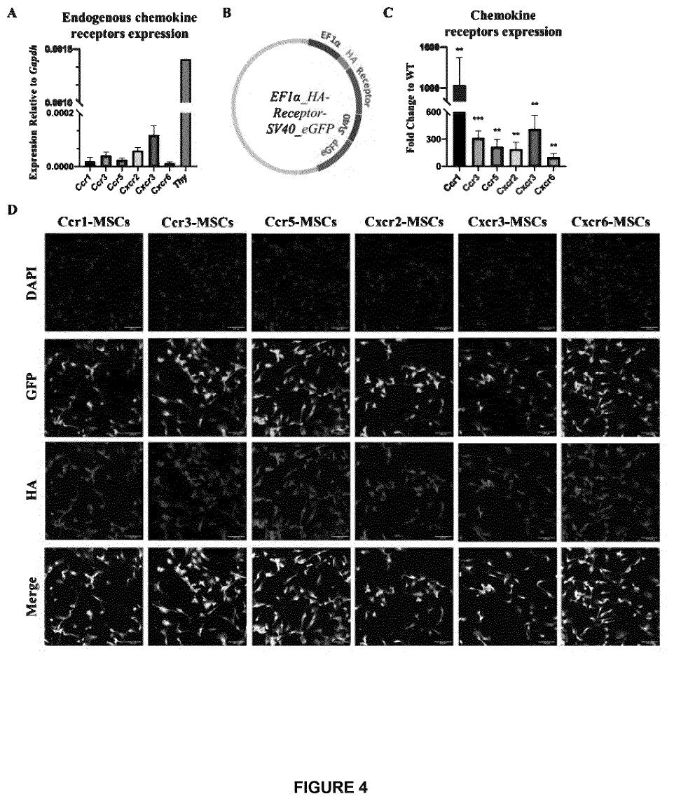

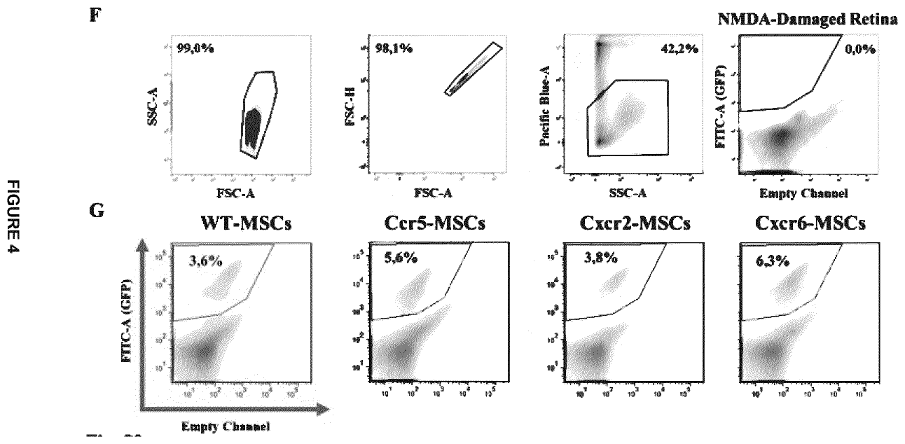

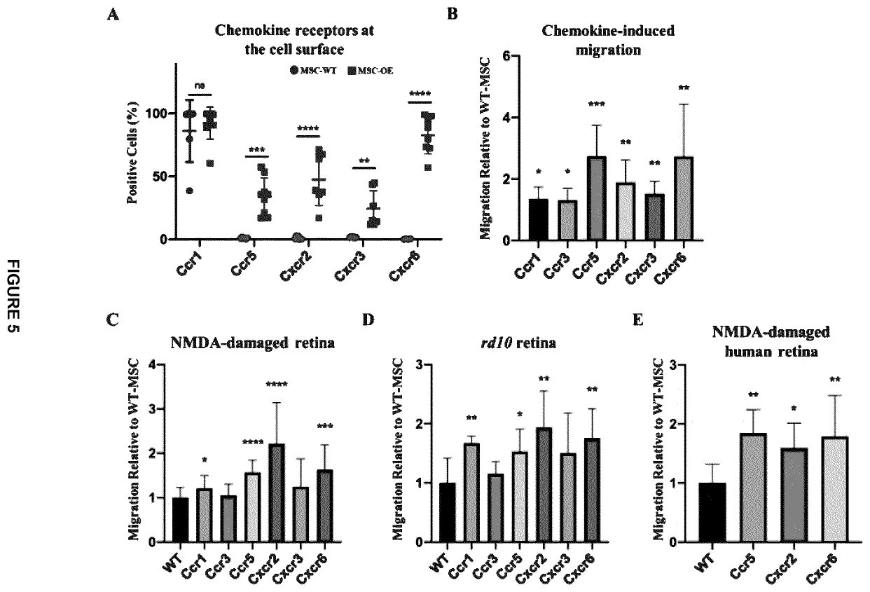

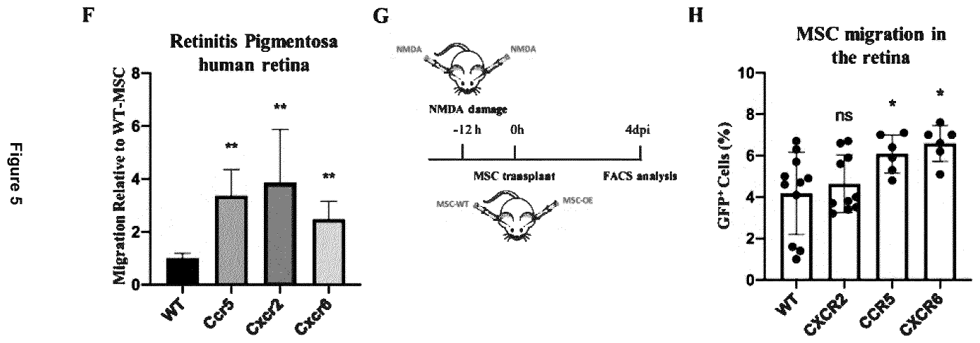

Generating and characterizing MSCs that over-express specific CC and CXC chemokine receptors Next, the endogenous expression levels of Ccr1, Ccr3, Ccr5, Cxcr2, Cxcr3 and Cxcr6 in MSCs were profiled. In accordance with published literature (see e.g. Shen et al., (2018), Cell Death Discovery, 9(2):229), it was found that MSCs expressed chemokine receptors at an almost negligible level when compared to the mesenchymal marker Thy (FIG. 4A). Thus, six distinct over-expressing (OE-MSC) lines were generated, one for each of the aforementioned receptors. Generating OE-MSCs was achieved via lentiviral infection of constructs containing both the HA-tagged receptor and an eGFP reporter (EF1α_HA-Receptor-SV40_eGFP; FIG. 4B). The resulting OE-MSC lines were characterised using multiple approaches. First, it was confirmed that receptor mRNAs were upregulated (FIG. 4C). Second, immunofluorescence was used to confirm the expression of the GFP marker and HA-tag (FIG. 4D). Third, FACS was used to check for the presence of the receptors at the cell surface (FIG. 5A). Finally, it was determined that the receptors were functional. To this end transwell assays were performed to assess the migration of WT- and OE-MSCs towards chemically-defined chemokine gradients (FIG. 5B). In this experiment the following receptor-chemokine pairs were tested: Ccr1/Ccr3/Ccr5-Ccl5; Cxcr2-Cxcl1; Cxcr3-Cxcl10; and Cxcr16-Cxcl6. In all cases, it was found that OE-MSCs migrated significantly more than their WT counterparts (FIG. 5B). This indicated that the exogenously expressed receptors can respond to chemokine gradients established by their cognate ligands.

Collectively, these results demonstrated that OE-MSCs expressed relatively high levels of the transduced chemokine receptors, and that such receptors were functional and correctly localised at the cell surface.

### Example 4

OE-MSCs Display Enhanced Migration

Next, migration of OE-MSCs in transwell assays was investigated using media conditioned by degenerating retina. The results showed that over-expression of Ccr1, Ccr5, Cxcr2 or Cxcr6 could significantly increase the migration of MSCs towards the media conditioned by both NMDA-damaged (FIG. 5C) and rd10 retina (FIG. 5D).

Based on these results, the migration of OE-MSC lines overexpressing Ccr5, Cxcr2 or Cxcr6 was investigated in chemotactic assays with media conditioned by both NMDA-damaged and RP-affected human retina. The chemokine receptor Ccr5 was chosen in this study rather than Ccr1, because the majority of MSCs endogenously express Ccr1 (see FIG. 5A). MSCs from all three overexpressing lines migrated more extensively than WT-MSCs, in both degenerative models (FIGS. 5E, 5F).

Next, the migration of Ccr5, Cxcr2 and Cxcr6 OE-MSCs in vivo was investigated. MSCs were transplanted intravitreally at 12 hour post-NMDA injection. After four days from transplantation (4 dpi), animals were sacrificed, and the percentage of MSCs in the retina was quantified by flow cytometry (FIG. 5G).

The results showed that over-expression of either Ccr5 or Cxcr6 alone led to a significant increase in the percentage of GFP⁺ MSCs in the retina (FIG. 5H; and FIGS. 4F, 4G).

### Example 5

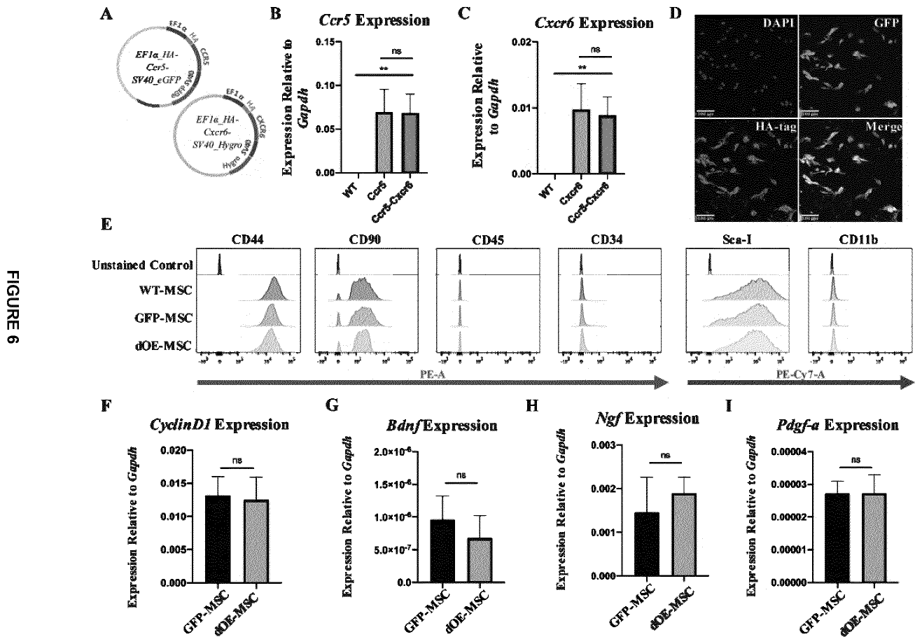

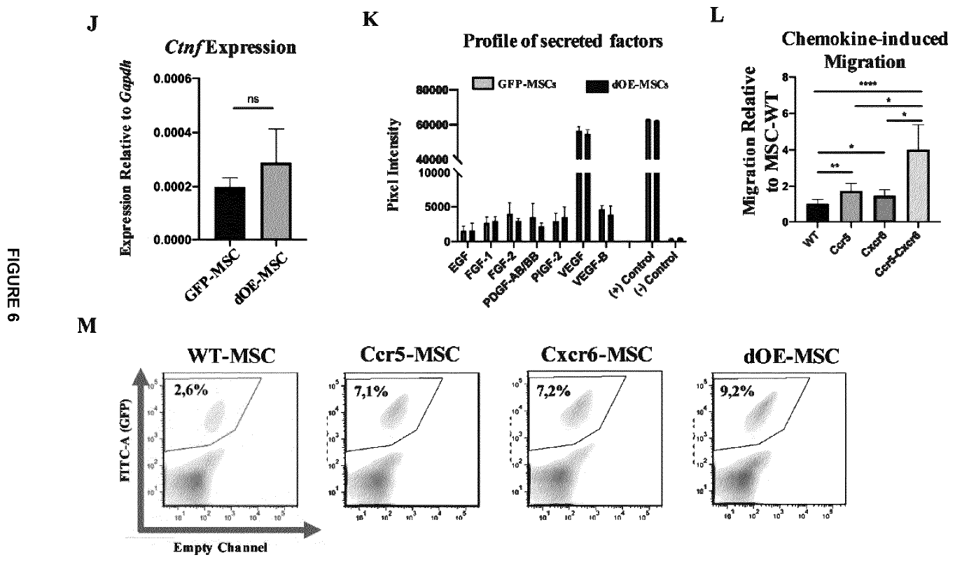

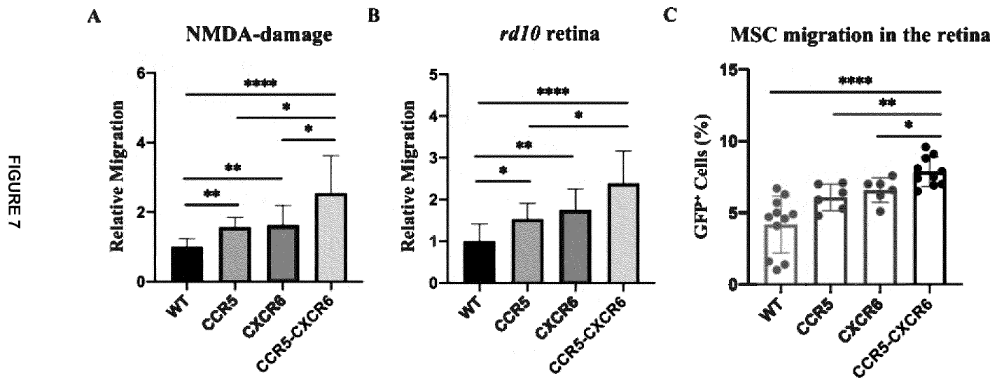

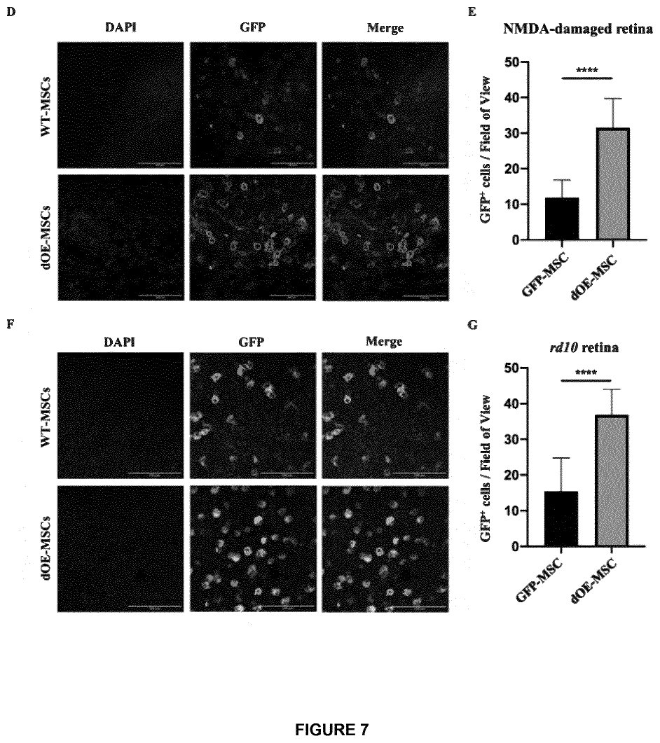

Combined over-expression of chemokine receptors further enhances migration of MSCs Since transplanted OE-MSCs overexpressing either Ccr5 or Cxcr6 showed significantly increased migration in vivo, combined over-expression of the two receptors was tested to investigate whether this would lead to a further improvement in cell migration (and integration) into damaged retina. To test this a double over-expressing (dOE) MSC line was generated. This was achieved via simultaneous infection of MSCs with two lentiviral constructs, each one driving expression of a single receptor (FIG. 6A). First, it was ensured that dOE-MSCs were over-expressing both Ccr5 and Cxcr6 (FIGS. 6B, 6C). Of note, mRNA levels of Ccr5 and Cxcr6 in dOE-MSCs were found to be comparable to that of single expressing OE-MSC lines (i.e. Ccr5-MSC and Cxcr6-MSC). Second, it was confirmed by immunofluorescence that the cells expressed the GFP and the HA tags (FIG. 6D). Third, flow cytometry was performed to ensure that the phenotype of GFP- and dOE-MSCs had not been altered due to lentiviral infection. It was found that WT-, GFP- and dOE-MSCs expressed the same stem cell surface markers. All populations were positive for the mesenchymal markers CD44, CD90 and Sca-I and negative for CD45, CD34 and CD11 b (FIG. 6E). Additionally, since chemokine signaling can be involved in the regulation of cell proliferation, it was confirmed that GFP- and dOE-MSCs showed comparable expression of cyclin D1 (FIG. 6F), which is a marker of proliferative cells. The expression of multiple factors that are known to mediate the neuroprotective paracrine effects of MSCs in the retina were also measured: brain-derived neurotrophic factor (Bdnf), nerve growth factor (Ng/), platelet-derived growth factor alpha (Pdgf-a) and ciliary neurotrophic factor (Cntf). Any statistically significant difference in the expression of such neurotrophic factors between GFP- and dOE-MSCs were identified (FIGS. 6G, 6H, 6I and 6J). It was also demonstrated that the secretory profiles of cells were comparable by analysing the media conditioned by either GFP- or dOE-MSCs. For example, it was determined that there were no significative changes in the secretion of EGF, FGF-1, PDGF, PIGF-2, and VEGF (FIG. 6K). Lastly, chemotactic assays towards a chemically-defined gradient of Ccl5 and Cxcl16 were performed to ensure that exogenously expressed Ccr5 and Cxcr6 were functional (FIG. 6L). In addition, the migration of dOE-MSCs towards medium conditioned by degenerating retina was tested. It was found that MSCs expressing both chemokine receptors exhibited desirable migration along a Ccl5 and Cxcl16 gradient. Furthermore, dOE-MSCs demonstrated enhanced migration in comparison to WT-MSCs and MSCs expressing either receptor alone (FIGS. 7A, 7B). Finally, in vivo migration of dOE cells was investigated by FACS analysis after transplantation. This showed that the number of GFP⁺ cells in the retina was highest for MSCs expressing both Ccr5 and Cxcr6 (FIG. 7C and FIG. 6M).

To investigate long-term migration and integration of genetically-modified cells, either WT- or dOE-MSCs were transplanted into NMDA-damaged retina following intravitreal transplantation and P18 rd10 retina following subretinal transplantation. Animals were sacrificed 3 weeks post-transplantation and the number of GFP⁺ MSCs in retinal flat mounts was counted (FIGS. 7D, 7E, 7F and 7G). It was found that, compared to the WT-controls, the number of dOE-MSCs was 2.7 and 2.4 times higher in NMDA-damaged and rd10 retina, respectively (FIGS. 7E, 7G). Interestingly, the presence of GFP⁺ cells in areas that were not visibly damaged by the NMDA was not observed, as evidenced by a high number of βIII-tubulin⁺ cells (FIG. 8A), suggesting that MSCs are selectively attracted to the area of damage.

To exclude the possibility that the observed GFP⁺ cells were retinal cells that had phagocytosed apoptotic MSCs, an in vitro assay was performed to investigate the dynamics of GFP degradation following phagocytosis by retinal pigmented epithelial (RPE) cells (FIGS. 8B, 8C and 8D). In this study it was established that RPE cells could phagocytose and rapidly clear apoptotic bodies derived from GFP-MSCs. RPE cells transiently became GFP-positive, with a peak at 1-3 hours from the exposure to the apoptotic bodies. Within 24 hours, most RPE cells had already degraded the internalised GFP and appeared to be GFP-negative. After 72 hours, the entire RPE population was negative for GFP expression (FIGS. 8B, 8C and 8D). These results suggested that the GFP coming from apoptotic MSCs would be rapidly cleared by phagocytic cells of the retina immediately after transplantation. Thus, it is very unlikely that the GFP-positive cells observed in the tissue at 3 wpi are indeed phagocytic retinal cells. To further support these observations, GFP-MSCs and dOE-MSCs were also transplanted intra vitreously in NMDA-damaged transgenic mice ubiquitously expressing DsRed. In this model, all retinal cells are positive for DsRed. Therefore, if a DsRed⁺ retinal cell phagocytosed a GFP⁺ MSC, then the two fluorescent markers would co-localise. Alternatively, the co-localisation of the two markers might be the result of cell fusion. Indeed, it has previously been shown that retinal cells can fuse with transplanted bone marrow-derived stem cells (e.g. Sanges et al., (2016), J. Clin. Investig., 126(8):3104-16). In these studies it was founds that at 3 wpi, less than 30% of the GFP⁺ cells were DsRed⁺ (FIGS. 8E, 8F and 8G). This might indicate that transplanted MSCs had undergone apoptosis and that apoptotic bodies had been phagocytosed by retinal cells shortly before the mice were sacrificed. Alternatively, and more likely, cell fusion might have occurred; indeed, we found that some GFP⁺-DsRed⁺ cells contained two separated nuclei (FIG. 8G). Either way, importantly, more than 70% of the GFP-positive cells were DsRed-negative in the long-term (FIG. 8E). In addition, there was no significant difference in the DsRed⁺-GFP⁺/GFP⁺ ratio between GFP- and dOE-MSCs (DsRed⁺-GFP⁺/GFP⁺_{GFP-MSC}=27,2%; DsRed⁺-GFP⁺/GFP⁺_{dOE-MSC}=29,6%).

In conclusion, even though the possibility of short-term phagocytosis events or cell fusion for a percentage of the transplanted cells cannot be excluded, these results suggest that approx. 70% of the GFP-expressing cells observed in the long-term are indeed derived from the transplanted MSCs.

### Example 6

Transplanted Genetically-Modified MSCs Rescue Retinal Degeneration

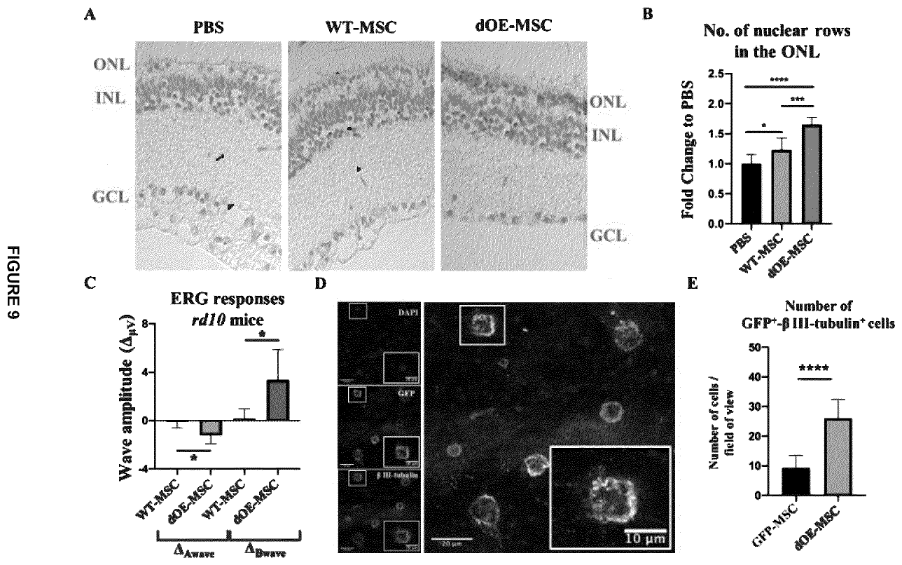

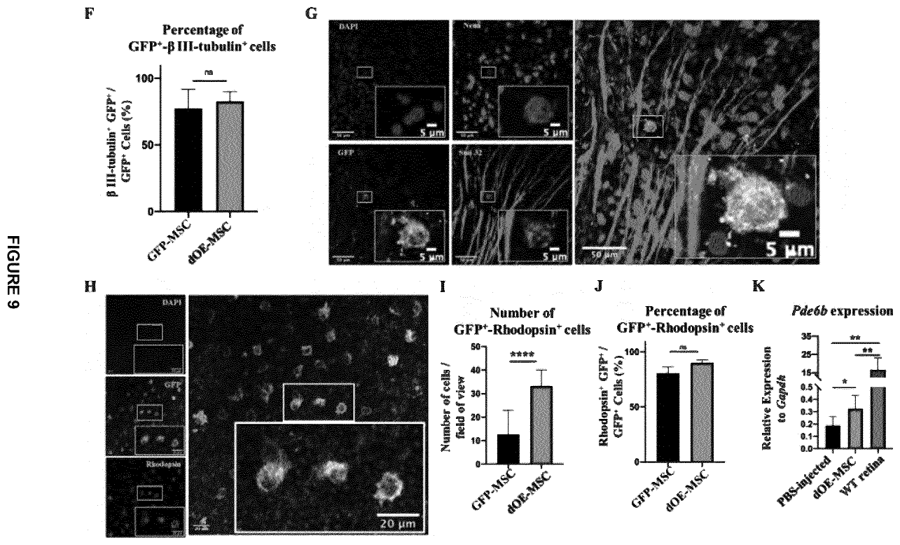

As noted above, MSCs have been previously demonstrated to rescue tissue degeneration through their paracrine activity (e.g. (Baraniak & McDevitt (2010), Regenerative Medicine, 5(1):121-43; Johnson et al., (2014), Brain, 137(Pt 2):503-19). To investigate the potential beneficial effects of dOE-MSCs transplantation, the number of rows of photoreceptor nuclei were counted in sections from rd10 mice that had received an injection of PBS in one eye, and transplantation of either WT- or dOE-MSCs in the other eye (FIGS. 9A and 9B). It was found that retinal sections from control (PBS-injected) eyes contained an average of 1.5 rows, which is indicative of severe photoreceptor degeneration. The number of rows was slightly increased following transplantation of MSCs. However, the outer nuclear layer (ONL) was significantly thicker when dOE-MSCs, rather than WT cells, had been transplanted (FIGS. 9A and 9B).

Scotopic electroretinographic (ERG) responses from dark-adapted rd10 animals were also recorded. Each animal had one eye treated with either WT- or dOE-MSCs; the other eye was used as an internal (PBS-injected) control. We found that the mice could be segregated into two groups. Animals in the first group (n_{WT}=5; n_{OE}=5; n_{WT+OE}=10) showed very small ERG amplitudes. In contrast, animals in the second group (n_{WT}=3; n_{OE}=4; n_{WT+OE}=9) had more prominent ERG amplitudes. The inventors hypothesise that these differences may be due to slower progression of the photoreceptor degeneration in the latter group. For both groups, the differences in A-wave and B-wave amplitudes (Δ_{A}, Δ_{B}) between control (PBS-injected) and experimental (WT/dOE-MSC-injected) eyes was measured. It was found that, compared to controls, eyes transplanted with MSCs expressing Ccr5 and Cxcr6 displayed a significant increase in both A- and B-wave amplitudes (FIG. 9C).

### Example 7

Transplanted MSCs Express Retinal-Specific Markers in the Long-Term

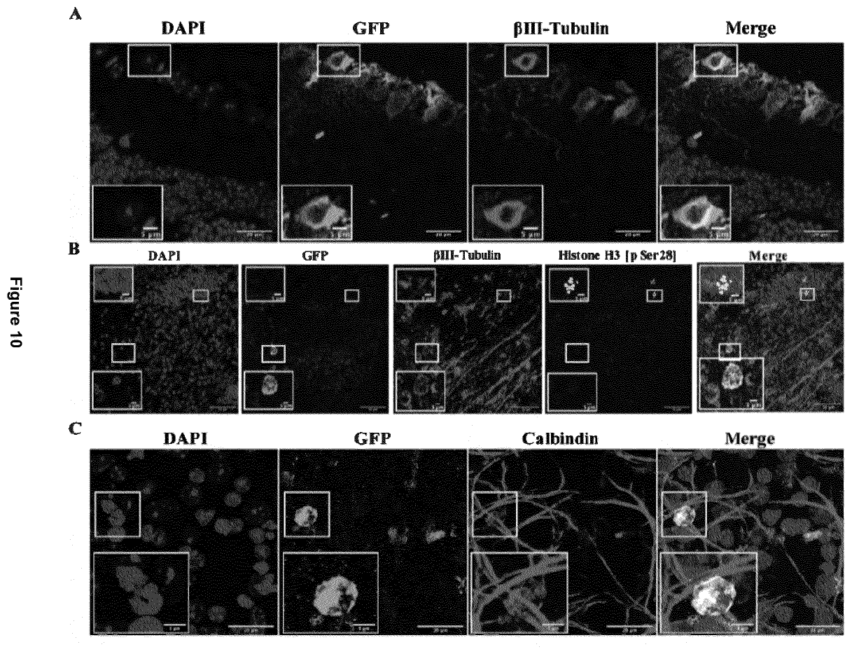

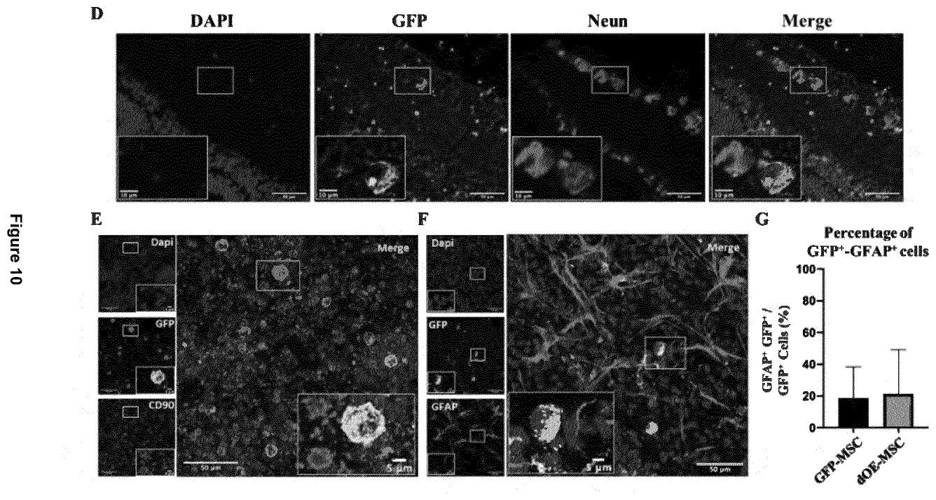

Even though they have been predominantly studied for their paracrine activity, MSCs possess a degree of plasticity that allows their conversion into a variety of cell types, including neuronal cells. In order to assess whether MSCs could change their phenotype after transplantation, retinal flat mounts from NMDA-treated animals were stained for βIII-tubulin, a neuron-specific marker expressed by ganglion cells and retinal interneurons. In these studies, it was found that most of the GFP⁺ MSCs were positive for βIII-tubulin and appeared integrated in the ganglion cell layer (GCL) (FIGS. 9D, 9E and 9F; FIG. 10A). βIII-tubulin is exclusively expressed in neurons within the CNS. Nonetheless, there is some evidence that it might also be expressed by other cell types (e.g. fibroblasts, keratinocytes and various cancer cell lines) during metaphase and anaphase (Jouhilahti et al., (2008), J. Hist. Cytochem., 56(12):1113-9). In this direction, retinal flat mounts were stained for βIII-tubulin and phospho-histone H3 (Ser10), a marker of mitotic cells. GFP-positive cells were positive for βIII-tubulin but negative for phospho-histone H3 (FIG. 10B), suggesting that MSCs transplanted into retina could indeed acquire expression of neuronal-specific markers in the long-term. To further support this hypothesis, stained retinal flat mounts and retinal sections were stained for additional neuronal markers. In this study, it was found that, 3 wpi, some of the GFP⁺ MSCs expressed Neun, Smi-32 and Calbindin (FIG. 9G; FIGS. 10C and 10D). Importantly, it was found that, 3 wpi, MSCs had lost expression of the mesenchymal marker CD90 (Thy) (FIG. 10E). Moreover, MSCs were largely negative for glial marker GFAP expression (FIG. 10F), with only 18.7% of the GFP-MSCs and 21.4% of the dOE-MSCs expressing GFAP 3 wpi (FIG. 10G).

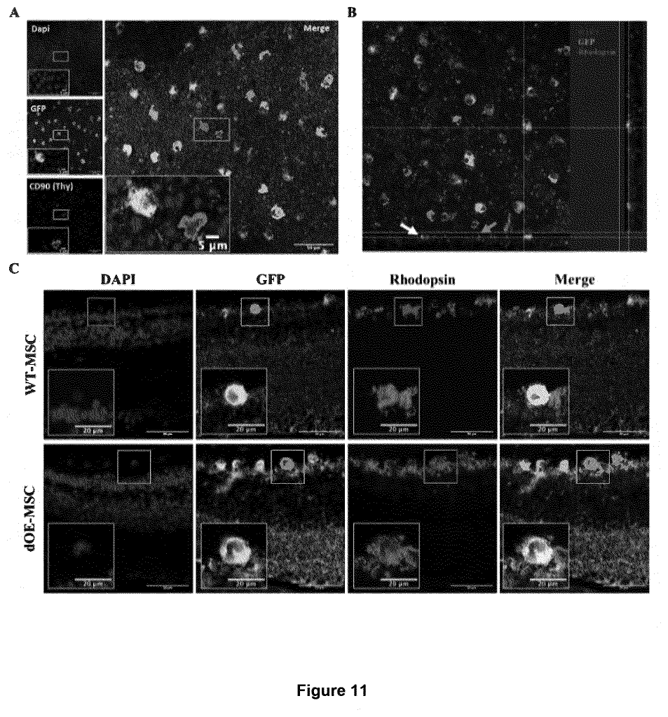

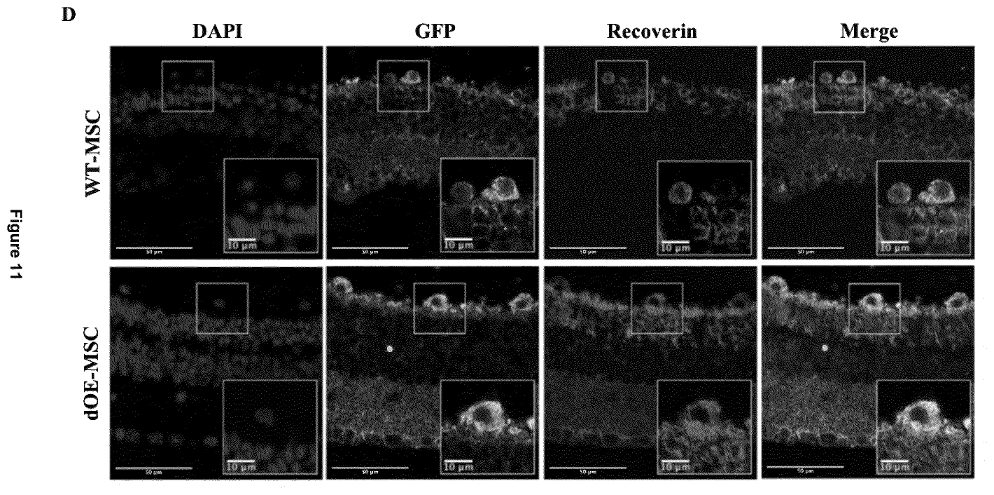

Similarly, in the rd10 retina, MSCs lost expression of CD90 (Thy) (FIG. 11A). The majority of the transplanted GFP⁺ MSCs were positive for the photoreceptor-specific markers Rhodopsin (FIGS. 9H, 9I, 9J; and FIG. 11C) and Recoverin (FIG. 11D). Moreover, the transplanted MSCs were apparently located within the same layer as Rhodopsin⁺ photoreceptors (FIG. S7B), although they did not fully integrate in the ONL, likely due to their large size. Compared to controls, the total number of GFP⁺/βIII-tubulin⁺ and of GFP⁺/Rhodopsin⁺ dOE-MSCs was significantly increased (FIG. 4E, I). Interestingly, however, we found that the proportion of cells that were positive for βIII-tubulin or Rhodopsin was comparable for WT- and dOE-MSCs (FIGS. 9F and 9J). This indicates that whilst the number of transplanted MSCs that migrate into the retina is dependent upon the expression of Ccr5 and Cxcr6, both WT- and dOE-MSCs, once integrated into the retina, can express retinal specific markers. RT-PCR analysis of rd10 retina at 3 wpi was also performed. It was found that transplanting dOE-MSCs could partially restore expression of the Pde6b gene (FIG. 9), further suggesting that partially integrated MSCs in the ONL can express genes characteristic of photoreceptors.

In conclusion, upon combined exogenous expression of Ccr5 and Cxcr6, transplanted MSCs can more efficiently migrate towards the damaged host retina. In agreement with published literature, such transplanted MSCs can then delay the death of neighboring retinal cells, most likely through their potent survival-promoting paracrine activity. At the same time, they can also acquire expression of genes characteristic of ganglion neurons and photoreceptors, highlighting the potential role that cell transdifferentiation may play in delaying retinal degeneration.

Other variations of the invention will be apparent to the skilled person without departing from the scope of the appended claims.

### DISCUSSION

In this work, CC and CXC inflammatory chemokines upregulated in two distinct models of retinal degeneration were profiled. Stem cells were then engineered that displayed improved migration toward media conditioned by degenerating retina from both mice and humans. In vivo, these cells could be efficiently chemoattracted and were demonstrated to beneficially integrate into host retina, thanks to the engagement of chemokine receptors, such as Ccr5 and Cxcr6 receptors, by a subset of the identified CC and CXC chemokines. Importantly, transplantation of MSCs expressing exogenous chemokine receptors, such as Ccr5 and/or Cxcr6 receptors, resulted in a beneficially thicker ONL, which was indicative of a rescue of the endogenous photoreceptor cells in a model of retinitis pigmentosa (RP).

MSCs transplantation thus appears to ameliorate the degenerative phenotype of retina via two distinct mechanisms.

First, there is evidence to suggest that transplanted MSCs may promote the survival of endogenous cells through their paracrine and neuroprotective activity (e.g. see Levkovitch-Verbin et al., (2010), Investigative Ophthalmology and Visual Science, 51(12):6394-6400). The neurotrophic factors secreted by MSCs can reduce inflammation, apoptosis and fibrosis while enhancing neuronal survival and differentiation (Tzameret et al., (2015), Stem Cell Research, 15(2):387-94). It has also been suggested that the neuroprotective and anti-inflammatory properties of MSCs may be mediated by extracellular vesicles (EVs) (see e.g. Keshtkar et al., (2018), Stem Cell Research & Therapy, 9(1):63). Indeed, MSC-derived EVs have been shown to reduce cell death and prevent apoptosis in numerous disease models, including bone and cartilage degeneration, neurological disorders, liver injury, kidney failure, muscle degeneration and cardiovascular diseases. Intravitreal administration of MSC-derived EVs has been shown to enhance functional recovery while decreasing neuro-inflammation and apoptosis in models of retinal ischemia (Mathew et al., (2019), Biomaterials, 197:146-160), glaucoma and autoimmune uveitis (Shigemoto-Kuroda et al., (2017), Stem Cell Reports, 8(5):1214-25). Interestingly, the protection conferred by EVs seems to be higher when multiple administrations are performed or when EVs are injected in high doses. In this direction, transplantation of OE-MSCs and dOE-MSCs with improved migratory capability over WT-MSCs could result in an enhanced rescue of the degenerative phenotype, due to the release of EVs close to the injury site/areas of damaged retina.

Second, it should also be noted that in the medium-to-long term, MSCs lose expression of the mesenchymal marker CD90 (Thy), while acquiring expression of retina-specific neuronal and glial markers. These results suggest—without being bound by theory—the possibility that transplanted MSCs may convert into functional retinal cell types and thereby replace lost cells. Electrophysiological responses measured at the level of individual cells might give useful insights in the future. Likewise, the membrane potentials characteristic of neurons, or their response to neurotransmitters, could be investigated.

There results also showed that intravitreally transplanted MSCs could reach the ganglion cell layer and integrate within the tissue. However, the data suggests that subretinally transplanted MSCs may not fully integrate within the photoreceptor nuclear layers, most likely due to their large size. In this regard, cultured MSCs are over 20 μm in diameter, which might limit or impede their penetration through the outer limiting membrane and inside the layer of tightly compacted photoreceptors. Therefore, it is envisaged that therapeutic approaches specifically aiming at cell replacement in the ONL might particularly employ genetically-modified photoreceptor precursor cells expressing exogenous chemokine receptors; such as Ccr5, Cxcr6, Ccr1 and/or Cxcr2. Strategies facilitating the penetration of such genetically-modified cells through the outer limiting membrane are envisaged to further enhance cell integration into damaged ONL. On the basis of these considerations, the inventors hypothesise that the paracrine activity of transplanted MSCs may be largely responsible for the ameliorated degenerative phenotype, and that all such therapeutic effects are enhanced by the enhanced migration and integration of genetically-modified cells according to this disclosure towards and into damaged/diseased retina.

Significantly, only a small percentage of the total MSC population expresses either Ccr5 (<1%) or Cxcr6 (<0.5%) on their surface. The results described herein are consistent with published literature, and highlights the limited repertoire of chemokine receptors that MSCs endogenously express. It is also important to consider that MSC chemokine receptor profile may be sensitive to time in culture (89). More specifically, prolonged ex vivo cell culturing and expansion might lead to substantial downregulation of chemokine receptor expression. Genetic modification of cultured MSCs would thus allow to overcome such problems.

In the past, CC and CXC chemokines have been shown to synergise to increase leukocyte recruitment to inflamed tissues (Proudfoot & Uguccioni (2016), Frontiers in Immunology, 7:183). The specific mechanisms regulating such synergy have not been clearly elucidated yet. However, it has been suggested that synergistic effects could result from receptor heterodimerisation or chemokine cooperation at the level of intracellular signal transduction. Either way, synergistic interactions between chemokines that are concomitantly released can contribute to the enhancement and the fine-tuning of inflammatory responses. Genetically-modified cells according to this disclosure that express more than one exogenous chemokine receptor may take advantage of the existence of such cooperative mechanisms to boost the recruitment of exogenously transplanted MSCs via combined expression chemokine receptors, such as Ccr5 and Cxcr6.

Notably, the subsets of upregulated chemokines were strikingly similar and largely overlapping in both of the degenerative models tested in this study. Interestingly, inflammatory responses of the retina are mainly orchestrated by Muller glial cells (MGCs), retinal pigment epithelial cells (RPECs) and activated microglia, independent of the cell type that is initially damaged (Rutar et al., (2015), J. Neuroinflammation, 12:8). For instance, Ccl5 in the P18 rd10 mouse retina is produced by microglial cells and MGCs of the INL (Zeng et al., (2006), Investigative Ophthalmology & Visual Science, 47(13):5772). The existence of these highly comparable, site-(rather than disease-) specific patterns of chemokine upregulation may desirably make the therapeutic uses and methods, as well as the genetically-modified cells and pharmaceutical compositions disclosed herein widely applicable to different diseases and disorders of the eye and retina. For example, the genetically-modified cells of the invention may therefore have utility in the treatment of patients with other types of retinopathies, such as AMD or optic neuropathies.

A recent study reported that homogenates from injured brains play a repulsive role on MSC migration (Andrzejewska et al., (2020), Theranostics, 10(15):6615-28). Here, we used conditioned media rather than tissue homogenates for ex vivo transwell assays. By doing so, we have specifically investigated migration of MSCs towards the soluble factors released by the damaged retina, which, in vivo, are responsible for chemoattraction. When tissue homogenates are used, instead, intracellular factors are also released which might have an effect on the investigations.

Survival and integration of transplanted cells can be affected by the administration route used. Systemic administration by intravenous injection is commonly used for MSCs, as it is safe and allows for infusion of a large number of cells. However, it generally shows low efficiency with respect to homing to the injury site: for example, MSCs may get trapped in the lungs and cleared out of the patient (se e.g. Assis et al., (2010), Cell Transplantation, 19(2):219-230). Additionally, even if they could escape lung entrapment, their migration into the retina would be further inhibited by the blood-retinal barrier. This could explain why systemically transplanted MSCs can be short-lived and why they generally fail to reach the retina and to exert neuroprotective effects (Johnson et al., (2010), Investigative Ophthalmology & Visual Science, 51(4):2051-9). In contrast, direct delivery into tissues can allow MSCs to escape systemic clearance and to persist locally up to several weeks (Boulland et al., (2012), Cell Transplantation, 21(8):1743-59). As a consequence, in the context of the treatment of eye/retinal diseases, disorders and damage, MSCs are preferably administered via either intravitreal or subretinal injections. In this study, locally transplanted MSCs could survive at least three weeks in order to exert therapeutic effects which may extend significantly beyond this period. Of note, the suitability of each administration route may vary depending on the type and on the extent of tissue damage. As a general rule, intravitreal injection is preferred when ganglions and/or inner neuronal layer (INL) neuronal cells are damaged, whereas subretinal administration is the standard route in the context of photoreceptor loss.

The strategies for therapeutic uses and methods disclosed herein are considered to have an extremely high degree of adaptability and versatility, as they could potentially be applied to any type of transplanted cells. As a significant example, it is envisaged that the homing/localisation of genetically-modified retinal and/or photoreceptor precursors cells, which are inherently good at differentiating into mature retinal neurons, but display very poor migratory capabilities may be significantly enhanced by way of the invention.

The success of the approaches disclosed herein is likely dependent on the secretion of chemotactic factors into the vitreous cavity. Importantly, this is known to happen during retinal degeneration in human patients (e.g. Yoshimura et al., (2009), PLoS One, 4(12):e8158). Indeed, there seems to be a correlation between the number of inflammatory cells recruited in the vitreous cavity and the visual function of the patient: the higher the former, the lower the latter. For instance, CXCL16 levels in the aqueous humor of wet AMD patients positively correlate with lesion size (Liu et al., (2016), Molecular Vision, 22:352-61). Importantly, in human RP patients, inflammation is chronic. Even though stronger inflammatory reactions are generally found in younger patients with active disease processes, the inflammatory state continues even after photoreceptor loss (Yoshida et al., (2013), Ophthalmology, 120(1):100-5). Such persistent inflammation means that the levels of chemokines released by the tissue will be elevated throughout the patient's lifespan. This is consistent with the results presented herein, which showed that conditioned medium from the retina of elderly RP patients can strongly chemoattract the genetically-modified MSCs of the invention. In this regard, the samples used in the above studies were obtained from patients that had already passed away at an age of at least 65 and, therefore, had already gone through the acute phase of photoreceptor loss.

It is further envisaged that genetically modified cells according to the invention, such as OE-MSC lines, may particularly be generated via AAV infection, rather than lentiviral vectors (as used in Examples herein), because lentiviral vectors may randomly integrate into the genome, with the risk of potentially harmful and/or undesirable side effects, such as genetic mutations. AAV vectors have a specific site of integration, which may provide for a safer and more efficacious genetically-modified cell for use in therapy/a clinical setting. Indeed, a number of valid alternatives to lentiviral infection are available, including Sendai viruses (SeV) and also adenoviral (AV) vectors. Alternatively, non-viral systems (e.g. naked DNA or synthetic mRNAs) could also or alternatively be used (Hardee et al., (2017), Genes (Basel), 8(2):65).

### CONCLUSIONS

To study chemokine-mediated migration of genetically-modified cells the inventors used two distinct degeneration models. First, a pharmacological model, N-methyl-D-aspartate (NMDA)-induced excitotoxicity. NMDA induces acute loss of ganglion and amacrine neurons (see e.g. Sabel et al., (1995), Experimental Brain Research, 106(1):93-105; Sucher et al., (1997), Vision Research, 37(24):3483-93). In parallel, the rd10 mouse model of autosomal recessive retinitis pigmentosa (RP) was used. The rd10 mouse carries a missense point mutation in exon 13 of the rod-specific β subunit of the cGMP-phosphodiesterase (Pde6b) gene (e.g. Chang et al., (2002), Vision Research, 42(4):517-25). Pde6b is involved in the phototransduction cascade, and its absence induces progressive loss of both rod and cone photoreceptors. The peak of retinal inflammation in response to cellular damage and photoreceptor loss occurs at post-natal day 18 (P18) (e.g. Chang et al., (2002), Vision Research, 42(4):517-25). Additionally, experiments were performed using human retina isolated from deceased donors and cultured ex vivo. Retina cultured under control conditions were compared either to retina exposed to NMDA or to retina of deceased patients affected by RP.

Significantly, the inventors identified damage-dependent chemokines that are secreted by degenerating retina, and demonstrated that these chemokines function as chemoattractants for MSCs. Moreover, the inventors showed that expression of exogenous Ccr5 and Cxcr6 in MSCs significantly improved migration of MSCs both ex vivo and in vivo. This ameliorated neuronal death and improved electrophysiology response. In conclusion, this study shows that genetic manipulation of MSCs can significantly advance efforts to optimise cell therapy-based regenerative approaches.

Cell therapy approaches hold great potential for treating retinopathies, which are currently incurable. This study addresses the problem of inadequate migration and integration of transplanted cells into the host retina. To this end, the inventors have identified the chemokines that were most upregulated during retinal degeneration and that could chemoattract therapeutic cells, such as genetically-modified mesenchymal stem cells (MSCs). The results were observed using a pharmacological model of ganglion/amacrine cell degeneration and a genetic model of retinitis pigmentosa, from both mice and human retina. Remarkably, MSCs over-expressing Ccr5 and Cxcr6, which are receptors bound by a subset of the identified chemokines, displayed improved migration after transplantation in the degenerating retina. They also led to enhanced rescue of cell death and to preservation of electrophysiological function. Overall, it was demonstrated that chemokines released from the degenerating retina can drive migration of transplanted stem cells, and that over-expression of chemokine receptors can improve cell therapy-based regenerative approaches.

It can be appreciated that the concepts as discussed and exemplified in relation to the treatment of retinal diseases can be applied to the treatment of tissue damage and/or degenerative diseases in other contexts. As described above in relation to the retina, it is possible to determine chemokine involvement in damaged or degenerative tissues. This is contemplated to allow for the determination of which chemokine receptors can be exogenously expressed in therapeutic stem cells for the treatment of such tissues. For example, stem cells expressing Cxcr2 are contemplated to be of enhanced use in the treatment of disorders of skin and mucosal surfaces, such as epidermolysis bullosa or radiation-induced oral mucositis (Alexeev et al., (2016), Stem Cell Research & Therapy, 7(1):124; Shen et al., (2018), Cell Death Discovery, 9(2):229). Ccr1-expressing cells are contemplated to be of use in the treatment of damaged myocardium, such as infarcted myocardium (J. Huang et al. (2010) Circ Res 106, 1753-1762). To conclude, the inventions disclosed herein provide viable approaches to the challenge of achieving effective delivery and engraftment at the site of injury, and it show that genetic manipulation of stem cells prior to transplantation may provide for novel stem cell therapies for various tissues, in particular for the eye, especially for the treatment of retinopathies, and for achieving visual restoration.

In any embodiments of the invention, the exogenous nucleic acid(s) encoding chemokine receptors can include one or more of SEQ ID NOs: 87 to 116, which represent exemplary coding sequences for human CCR1 (SEQ ID NO: 87); human CCR3 (SEQ ID NOs: 88 to 95); human CCR5 (SEQ ID NOs: 96 to 98); human CXCR2 (SEQ ID NOs: 99 to 104); human CXCR6 (SEQ ID NOs: 105 to 108); mouse CCR1 (SEQ ID NO: 109); mouse CCR3 (SEQ ID NOs: 110 to 111); mouse CCR5 (SEQ ID NOs: 112 to 113); mouse CXCR2 (SEQ ID NOs: 114 to 115); or mouse CXCR6 (SEQ ID NO: 116). Variants, mutants and fragments of these sequences are also contemplated in this regard, where such sequences encode polypeptides having the effect of the relevant chemokine receptors. For example, the exogenous nucleic acids may have a sequence that is 80%, 85%, 90%, 95%, 98%, 99%, or 100% similar or identical to any one or more of SEQ ID NOs 87 to 116.

### CLAUSES

Various aspects and embodiments of the disclosure are defined in the following clauses.
1. A genetically-modified stem cell comprising an exogenous nucleic acid encoding a chemokine receptor, wherein the exogenous nucleic acid is operably linked to at least one promoter and/or enhancer sequence for expression of the chemokine receptor in the genetically-modified stem cell.
2. The genetically-modified stem cell of Clause 1, which comprises one or more exogenous nucleic acid, each of the one or more exogenous nucleic acid encoding a chemokine receptor selected from a group consisting of:
   (i) Ccr5, Cxcr6, Ccr1, Cxcr2 and/or Ccr3;
   (ii) Ccr5, Cxcr6, Ccr1 and/or Cxcr2; or
   (iii) Ccr5 and/or Cxcr6.
3. The genetically-modified stem cell of Clause 1 or Clause 2, which comprises an exogenous nucleic acid encoding the Ccr5 chemokine receptor and/or an exogenous nucleic acid encoding the Cxcr6 chemokine receptor.
4. A genetically-modified stem cell comprising an exogenous nucleic acid, wherein the exogenous nucleic acid is capable of causing expression or causing an increase in expression of an endogenous chemokine receptor in the genetically-modified stem cell, wherein the endogenous chemokine receptor is not expressed in the unmodified stem cell, or is expressed at a lower level in the unmodified stem cell.
5. The genetically-modified stem cell of Clause 4, wherein the exogenous nucleic acid encodes a polypeptide capable of causing expression or causing an increase in expression of an endogenous chemokine receptor in the genetically-modified stem cell; and wherein the exogenous nucleic acid is operably linked to at least one promoter and/or enhancer sequence for expression of the polypeptide in the genetically-modified stem cell; optionally wherein the exogenous nucleic acid encodes a polypeptide involved in gene editing; for example, a CRISPR-associated nuclease or zinc finger nuclease.
6. The genetically-modified stem cell of Clause 4 or Clause 5, wherein the endogenous chemokine receptor is selected from one or more chemokine receptor selected from a group consisting of:
   (i) Ccr5, Cxcr6, Ccr1, Cxcr2 and/or Ccr3;
   (ii) Ccr5, Cxcr6, Ccr1 and/or Cxcr2; or
   (iii) Ccr5 and/or Cxcr6.
7. The genetically-modified stem cell of any of Clauses 4 to 6, wherein the exogenous chemokine receptor is the Ccr5 chemokine receptor and/or the Cxcr6 chemokine receptor.
8. The genetically-modified stem cell of any preceding Clause, wherein the exogenous nucleic acid encodes a human chemokine receptor sequence, or a mouse chemokine receptor sequence
9. The genetically-modified stem cell of any preceding Clause, wherein the stem cell is selected from the group consisting of: pluripotent stem cells, multipotent stem cells and unipotent stem cells.
10. The genetically-modified stem cell of any preceding Clause, wherein the stem cell is a hybrid pluripotent cell, optionally wherein the hybrid pluripotent cell is selected from the group consisting of a fusion between a Muller Glia cell and a bone marrow derived stem cell, a fusion between an MSC or bone marrow derived stem cells with a neuron cell or a precursor cell, or a fusion between an iPSC-derived progenitor cell with a Muller Glia cell.
11. The genetically-modified stem cell of any preceding Clause, wherein the stem cell is an embryonic stem cell (ES cell), pluripotent stem cell (PS cell), induced pluripotent stem cell (iPS cell), mesenchymal stem cell (MSC), bone marrow-derived stem cell, retinal progenitor cell or photoreceptor precursor cell.
12. The genetically-modified stem cell of any preceding Clause, wherein the stem cell is a photoreceptor precursor cell, horizontal cell precursor, bipolar cell precursor, amacrine cell precursor, ganglion cell precursor, retinal pigment epithelial precursor cell or Muller precursor cell.
13. The genetically-modified stem cell of any preceding Clause, wherein the stem cell is selected from the group consisting of: an induced pluripotent stem cell (iPS cell), a mesenchymal stem cell (MSC) and a photoreceptor precursor cell (such as a rod precursor cell or a cone precursor cell).
14. The genetically-modified stem cell of any preceding Clause, wherein the stem cell is a mesenchymal stem cell (MSC).
15. The genetically-modified stem cell of any preceding Clause, wherein the stem cell is:
   (i) a mesenchymal stem cell (MSC); or
   (ii) a photoreceptor precursor cell.
16. The genetically-modified stem cell of any preceding Clause, wherein the stem cell is a CD34+ stem cell.
17. The genetically-modified stem cell of any preceding Clause, wherein the stem cell is selected from the group consisting of: a human stem cell, a non-human primate stem cell, a pig stem cell, a dog stem cell, a cat stem cell, or a rodent stem cell (including a mouse stem cell, a hamster stem cell and a rabbit stem cell).
18. The genetically-modified stem cell of any preceding Clause, wherein the stem cell is a human stem cell, a non-human primate stem cell, or a mouse stem cell.
19. The genetically-modified stem cell of any preceding Clause, wherein the at least one promoter and/or enhancer sequence is an inducible promoter and/or enhancer sequence.
20. The genetically-modified stem cell of any of Clauses 1 to 18, wherein the at least one promoter and/or enhancer sequence is constitutively active.
21. The genetically-modified stem cell of any preceding Clause, wherein the exogenous nucleic acid is integrated into the genome of the genetically-modified stem cell.
22. The genetically-modified stem cell of Clause 21, wherein the exogenous nucleic acid is integrated into a predetermined position in the genome of the genetically-modified stem cell.
23. The genetically-modified stem cell of any preceding Clause, wherein the exogenous nucleic acid is comprised within a transposon system.
24. The genetically-modified stem cell of any of Clauses 1 to 20, wherein the exogenous nucleic acid is not integrated into the genome of the genetically-modified stem cell; for example, wherein the exogenous nucleic acid is episomal or extrachromosomal.
25. The genetically-modified stem cell of any of Clauses 1 to 20, wherein the exogenous nucleic acid is RNA, typically mRNA.
26. The genetically-modified stem cell of any preceding Clause, which is essentially free of exogenous viral genetic factors.
27. The genetically-modified stem cell of any of Clauses 1 to 23, wherein the exogenous nucleic acid is part of a proviral sequence; for example, a proviral sequence that is integrated into the stem cell genome.
28. The genetically-modified stem cell of Clause 23, wherein the proviral sequence is selected from the group consisting of: retroviruses (such as influenza, simian immunodeficiency virus (SIV), human immunodeficiency virus (HIV), lentivirus, and Moloney murine leukaemia); adenoviruses; adeno-associated viruses (AAV); sendai virus (SeV); herpes simplex virus (HSV); and chimeric viruses.
29. The genetically-modified stem cell of Clause 27 or Clause 28, wherein the proviral sequence is from:
   (i) an adeno-associated virus (AAV);
   (ii) a sendai virus (SeV);
   (iii) an AAV vector selected from the group consisting of: AAV1, AAV2, AAV3, AAV4, AAV5, AAV6, AAV6.2, AAV7, AAV8, AAV9, rhlO, AAVrh64RI, AAVrh64R2 and rh8 or variants thereof; or (iv) an AAV vector selected from AAV2/1 and AAV2/9 viral subtypes.
30. The genetically-modified stem cell of any preceding Clause, which comprises an endogenous or an exogenous expression cassette that expresses and secretes polypeptide molecules that maintain or enhance the activity, survival and/or number of cells in proximity to the genetically-modified stem cell.
31. The genetically-modified stem cell of any preceding Clause, which further comprises an exogenous nucleic acid encoding a neuroprotective factor, an anti-angiogenic factor and/or a fragment or variant thereof; optionally, wherein the neuroprotective factor comprises a GLP-1 peptide or fragment or variant thereof, and the anti-angiogenic factor comprises endostatin, or a fragment or variant thereof.
32. The genetically-modified stem cell of any preceding Clause, which is capable of secreting one or more endogenous proteins or peptides as paracrine factors; optionally, wherein the paracrine factor is selected from VEGF, IL6, IL8, GDNF, NT3, NeuroD1 and/or MCP1.
33. The genetically-modified stem cell of any preceding Clause, which further comprises an exogenous nucleic acid encoding a selectable or screenable marker which is operably linked to at least one promoter and/or enhancer sequence for expression of the selectable or screenable marker in the genetically-modified stem cell.
34. The genetically-modified stem cell of Clause 33, wherein the at least one promoter and/or enhancer sequence operably linked to the selectable or screenable maker comprises:
   (i) an inducible transcriptional responsive regulatory element for expressing of the screenable marker, for example, wherein the inducible transcriptional responsive regulatory element comprises a drug responsive regulatory element; or
   (ii) a constitutive promoter and/or enhancer combination.
35. The genetically-modified stem cell of any preceding Clause, which is isolated.
36. A pharmaceutical composition comprising the genetically-modified cell according to any of Clauses 1 to 35.
37. The pharmaceutical composition of Clause 36, further comprising at least one pharmaceutically acceptable diluent or excipient.
38. The pharmaceutical composition of Clause 36 or Clause 37, which is formulated as an injectable composition.
39. The pharmaceutical composition of any of Clauses 36 to 38, which is formulated as an aqueous solution or suspension.
40. The pharmaceutical composition of any of Clauses 36 to 39, which comprises between:
   (i) about 50,000 and 500,000 genetically-modified cells per μl;
   (ii) about 75,000 and 300,000 genetically-modified cells per μl; or
   (iii) about 100,000 and 200,000 genetically-modified cells per μl.
41. The pharmaceutical composition of any of Clauses 36 to 40, which is for intraocular administration; for example, for intravitreal administration or for subretinal administration.
42. A genetically-modified cell according to any of Clauses 1 to 35, or the pharmaceutical composition of any of Clauses 36 to 41, for use in a method for the treatment of tissue damage and/or a degenerative disease.
43. The genetically-modified cell for use according to Clause 42, or the pharmaceutical composition for use according to Clause 42, wherein the tissue damage and/or degenerative disease is a neurodegenerative disease.
44. The genetically-modified cell for use according to Clause 43, or the pharmaceutical composition for use according to Clause 43, wherein the neurodegenerative disease is selected from the group consisting of: Parkinson's Disease (PD), Huntington's disease (HD), Alzheimer's Disease (AD), frontotemporal dementia (FTD) and amyotrophic lateral sclerosis (ALS).
45. The genetically-modified cell for use according to Clause 42, or the pharmaceutical composition for use according to Clause 42, wherein the tissue damage and/or degenerative disease is damage to nervous tissue, typically to the spinal cord or brain, optionally ischemic damage.
46. The genetically-modified cell for use according to Clause 42, or the pharmaceutical composition for use according to Clause 42, wherein the tissue damage and/or degenerative disease is myocardial damage, typically resulting from myocardial infarction.
47. The genetically-modified cell for use according to Clause 42, or the pharmaceutical composition for use according to Clause 42, wherein the tissue damage and/or degenerative disease is a disorder of the skin, typically epidermolysis bullosa or radiation-induced oral mucositis.
48. The genetically-modified cell for use according to Clause 42, or the pharmaceutical composition for use according to Clause 42, wherein the tissue damage and/or degenerative disease is a disorder of the bones or joints, typically arthritis, arthrosis, pseudoarthrosis, infections, resection or trauma.
49. The genetically-modified cell for use according to Clause 42, or the pharmaceutical composition for use according to Clause 42, wherein the tissue damage and/or degenerative disease is a disorder of the lung, typically resulting from smoking, surgery, trauma, or infection
50. A genetically-modified cell according to any of Clauses 1 to 35, or the pharmaceutical composition of any of Clauses 36 to 41, for use in a method for the treatment of an eye disease or disorder; such as a retinopathy and/or damaged retina.
51. The genetically-modified cell for use according to Clause 50, or the pharmaceutical composition for use according to Clause 50, wherein the eye disease or disorder is caused by a degeneration of the retina (retinal disease), such as: retinitis pigmentosa (RP), macular degeneration (MD), age related macular degeneration (AMD or ARMD), macula edema due of other reasons, retinal vessel occlusions, diabetic retinopathy, glaucoma and other optic neuropathies, etc., and conditions associated therewith; Bassen-kornzweig syndrome, choroideremia, gyrate atrophy, Refsum syndrome, Usher syndrome, color blindness, blue cone monochromacy, achromatopsia, incomplete achromatopsia, oligocone trichromacy, Stargardt's Disease, Bardet-Biedl syndrome, Bornholm eye disease, Best's Disease and Leber's congenital amaurosis.
52. The genetically-modified cell for use according to Clause 51, or the pharmaceutical composition for use according to Clause 51, wherein the eye disease or disorder is selected from retinitis pigmentosa (RP), macular degeneration (MD), age related macular degeneration (AMD or ARMD), diabetic retinopathy, Stargardt's Disease and glaucoma.
53. The genetically-modified cell for use according to any of Clauses 50 to 52, or the pharmaceutical composition for use according to any of Clauses 50 to 52, wherein the method comprises administering the genetically-modified cell or pharmaceutical composition in combination with one or more additional therapeutic agents; optionally, wherein the genetically-modified cell or pharmaceutical composition is administered separately, sequentially or simultaneously with the one or more additional therapeutic agent.
54. The genetically-modified cell for use according to Clause 53, or the pharmaceutical composition for use according to Clause 53, wherein the one or more additional therapeutic agent is selected from the group consisting of: a neuroprotective factor, an anti-angiogenic factor and/or a fragment or variant thereof; optionally, wherein the neuroprotective factor comprises a GLP-1 peptide or fragment or variant thereof, and the anti-angiogenic factor comprises endostatin, or a fragment or variant thereof.
55. The genetically-modified cell for use according to any of Clauses 50 to 54, or the pharmaceutical composition for use according to any of Clauses 50 to 54, wherein the method comprises intraocular injection of the genetically-modified cell or the pharmaceutical composition into the intravitreal space or into the subretinal space.
56. The genetically-modified cell for use according to Clause 55, or the pharmaceutical composition for use according to Clause 55, wherein the method comprises intraocular injection of:
   (i) between about 50,000 and 2,000,000 genetically-modified cells;
   (ii) between about 57,000 and 1,500,000 genetically-modified cells;
   (iii) between about 100,000 and 1,000,000 genetically-modified cells;
   (iv) between about 150,000 and 750,000 genetically-modified cells;
   (v) between about 200,000 and 600,000 genetically-modified cells; or
   (vi) between about 250,000 and 500,000 genetically-modified cells.
57. The genetically-modified cell for use according to Clause 55 or Clause 56, or the pharmaceutical composition for use according to Clause 55 or Clause 56, wherein the method comprises intraocular injection of between about 1 and 50 μl of solution comprising the genetically-modified cells, between about 2 and 25 μl of solution comprising the genetically-modified cells, between about 3 and 20 μl of solution comprising the genetically-modified cells, between about 4 and about 15 μl of solution comprising the genetically-modified cells, or between about 5 and about 10 μl of solution comprising the genetically-modified cells.
58. The genetically-modified cell for use according to any of Clauses 50 to 57, or the pharmaceutical composition for use according to any of Clauses 50 to 57, wherein the method comprises intraocular injection of the genetically-modified cell or pharmaceutical composition into the intravitreal space or into the subretinal space.
59. The genetically-modified cell for use according to any of Clauses 50 to 58, or the pharmaceutical composition for use according to any of Clauses 50 to 58, wherein the genetically-modified cell is an MSC, and wherein the method comprises intraocular injection of the genetically-modified MSC or pharmaceutical composition comprising the genetically-modified MSC into the intravitreal space such that the genetically-modified MSC integrates into the ganglion cell layer (GCL) and/or cooperates with the ganglion cell layer (GCL) to repair and/or protect and/or increase the thickness of the GCL; and/or wherein the genetically-modified MSC integrates into the inner nuclear layer (INL) and/or cooperates with the inner nuclear layer (INL) to repair and/or protect and/or increase the thickness of the INL.
60. The genetically-modified cell for use according to any of Clauses 50 to 58, or the pharmaceutical composition for use according to any of Clauses 50 to 58, wherein the genetically-modified cell is an MSC, and wherein the method comprises intraocular injection of the genetically-modified MSC or pharmaceutical composition comprising the genetically-modified MSC into the subretinal space such that the genetically-modified MSC integrates into the outer nuclear layer (ONL) and/or cooperates with photoreceptor cells of the outer nuclear layer (ONL) to repair and/or protect and/or increase the thickness of the ONL.
61. The genetically-modified cell for use according to any of Clauses 50 to 58, or the pharmaceutical composition for use according to any of Clauses 50 to 58, wherein the genetically-modified cell is a photoreceptor precursor cell, and wherein the method comprises intraocular injection of the genetically-modified photoreceptor precursor cell or pharmaceutical composition comprising the photoreceptor precursor cell into the subretinal space such that the photoreceptor precursor cell integrates into the outer nuclear layer (ONL) and/or cooperates with photoreceptor cells of the outer nuclear layer (ONL) to repair and/or protect and/or increase the thickness of the ONL.
62. The genetically-modified cell for use according to any of Clauses 42 to 61, or the pharmaceutical composition for use according to any of Clauses 50 to 61, wherein the genetically-modified cell is adapted to secrete one or more endogenous proteins or peptides as paracrine factors so as to repair, maintain and/or protect subject cells; optionally, wherein the paracrine factor is selected from VEGF, IL6, IL8, GDNF, NT3, NeuroD1 and/or MCP1.
63. The genetically-modified cell for use according to any of Clauses 50 to 62, or the pharmaceutical composition for use according to any of Clauses 50 to 62, wherein the genetically-modified cell comprises an exogenous nucleic acid encoding Ccr5 and/or an exogenous nucleic acid encoding the Cxcr6.
64. The genetically-modified cell for use according to any of Clauses 50 to 63, or the pharmaceutical composition for use according to any of Clauses 50 to 63, wherein the method comprises subretinal implantation of a sheet of retinal pigmented epithelium (RPE) in combination with intraocular injection of a genetically-modified photoreceptor precursor cell into the subretinal space.
65. A method for treating or alleviating tissue damage and/or a degenerative disease in a subject, the method comprising:
   introducing into the subject a genetically-modified stem cell according to any of Clauses 1 to 35.
66. A method for treating or alleviating an eye disease or disorder (such as a retinopathy and/or damaged retina), in a subject, the method comprising:
   introducing into the subject's eye a genetically-modified stem cell according to any of Clauses 1 to 35.
67. The method according to Clause 66, wherein the eye disease or disorder is selected from the group consisting of: an eye disease or disorder caused by a degeneration of the retina (retinal disease), retinitis pigmentosa (RP), macular degeneration (MD), age related macular degeneration (AMD or ARMD), macula edema due of other reasons, retinal vessel occlusions, diabetic retinopathy, glaucoma and other optic neuropathies, etc., and conditions associated therewith; Bassen-kornzweig syndrome, choroideremia, gyrate atrophy, Refsum syndrome, Usher syndrome, color blindness, blue cone monochromacy, achromatopsia, incomplete achromatopsia, oligocone trichromacy, Stargardt's Disease, Bardet-Biedl syndrome, Bornholm eye disease, Best's Disease and Leber's congenital amaurosis.
68. The method according to Clause 66 or 67, wherein the eye disease or disorder is selected from retinitis pigmentosa (RP), macular degeneration (MD), age related macular degeneration (AMD or ARMD), diabetic retinopathy, Stargardt's Disease and glaucoma.
69. The method according to any of Clauses 65 to 68, wherein the method comprises administering the genetically-modified stem cell in combination with one or more additional therapeutic agents.
70. The method according to Clause 69, wherein the genetically-modified stem cell is administered separately, sequentially or simultaneously with the one or more additional therapeutic agent.
71. The method according to Clause 69 or Clause 70, wherein the one or more additional therapeutic agent is selected from the group consisting of: a neuroprotective factor, an anti-angiogenic factor and/or a fragment or variant thereof; optionally, wherein the neuroprotective factor comprises a GLP-1 peptide or fragment or variant thereof, and the anti-angiogenic factor comprises endostatin, or a fragment or variant thereof.
72. The method according to any of Clauses 66 to 71, wherein the method comprises intraocular injection of the genetically-modified stem cell; optionally, wherein the method comprises injecting the genetically-modified stem cell into the intravitreal space or into the subretinal space.
73. The method according to any of Clauses 66 to 72, wherein the method comprises intraocular injection of:
   (i) between about 50,000 and 2,000,000 genetically-modified cells;
   (ii) between about 57,000 and 1,500,000 genetically-modified cells;
   (iii) between about 100,000 and 1,000,000 genetically-modified cells;
   (iv) between about 150,000 and 750,000 genetically-modified cells;
   (v) between about 200,000 and 600,000 genetically-modified cells; or
   (vi) between about 250,000 and 500,000 genetically-modified cells.
74. The method according to any of Clauses 66 to 73, wherein the method comprises intraocular injection of between about 1 and 50 μl of solution comprising the genetically-modified cells, between about 2 and 25 μl of solution comprising the genetically-modified cells, between about 3 and 20 μl of solution comprising the genetically-modified cells, between about 4 and about 15 μl of solution comprising the genetically-modified cells, or between about 5 and about 10 μl of solution comprising the genetically-modified cells.
75. The method according to any of Clauses 66 to 74, wherein the genetically-modified cell is an MSC, and wherein the method comprises intraocular injection of the genetically-modified MSC into the intravitreal space such that the genetically-modified MSC integrates into the ganglion cell layer (GCL) and/or cooperates with the ganglion cell layer (GCL) to repair and/or protect and/or increase the thickness of the GCL; and/or wherein the genetically-modified MSC integrates into the inner nuclear layer (INL) and/or cooperates with the inner nuclear layer (INL) to repair and/or protect and/or increase the thickness of the INL.
76. The method according to any of Clauses 66 to 75, wherein the genetically-modified cell is an MSC, and wherein the method comprises intraocular injection of the genetically-modified MSC into the subretinal space such that the genetically-modified MSC integrates into the outer nuclear layer (ONL) and/or cooperates with photoreceptor cells of the outer nuclear layer (ONL) to repair and/or protect and/or increase the thickness of the ONL.
77. The method according to any of Clauses 66 to 76, wherein the genetically-modified cell is a photoreceptor precursor cell, and wherein the method comprises intraocular injection of the genetically-modified photoreceptor precursor cell into the subretinal space such that the genetically-modified photoreceptor precursor cell integrates into the outer nuclear layer (ONL) and/or cooperates with photoreceptor cells of the outer nuclear layer (ONL) to repair and/or protect and/or increase the thickness of the ONL.
78. The method according to any of Clauses 66 to 74, wherein the method comprises intraocular injection of a genetically-modified photoreceptor precursor cell into the subretinal space such that the genetically-modified photoreceptor precursor cell integrates into the outer nuclear layer (ONL) and/or cooperates with photoreceptor cells of the outer nuclear layer (ONL) to repair and/or protect and/or increase the thickness of the ONL;
   in combination with intraocular injection of a genetically-modified MSC into the intravitreal space such that the genetically-modified MSC integrates into the ganglion cell layer (GCL) and/or cooperates with the ganglion cell layer (GCL) to repair and/or protect and/or increase the thickness of the GCL.
79. The method according to Clause 77 or Clause 78, which comprises subretinal implantation of a sheet of retinal pigmented epithelium (RPE) in combination with intraocular injection of the genetically-modified photoreceptor precursor cell into the subretinal space.
80. The method according to any of Clauses 66 to 79, wherein the genetically-modified cell is adapted to secrete one or more endogenous proteins or peptides as paracrine factors so as to repair, maintain and/or protect subject cells; optionally, wherein the paracrine factor is selected from VEGF, IL6, IL8, GDNF, NT3 and/or MCP1.
81. The method according to any of Clauses 65 to 67, wherein the genetically-modified cell comprises an exogenous nucleic acid encoding Ccr5 and/or an exogenous nucleic acid encoding the Cxcr6.
82. A modified viral vector packaging a recombinant viral-based genome, for use in a method for treating a subject suffering from tissue damage and/or a degenerative disease, the method comprising: administering to the subject one or more of the modified viral vectors packaging a recombinant viral-based genome, wherein the recombinant viral-based genome comprises a cDNA insert encoding an exogenous chemokine receptor polypeptide; wherein, in use, the one or more of the modified viral vectors infects and causes increased expression of the chemokine receptor in target cells of the subject; optionally, wherein the cDNA insert is operably linked to at least one promoter and/or enhancer sequence for expression of the exogenous chemokine receptor polypeptide in target cells of the subject.
83. A modified viral vector packaging a recombinant viral-based genome, for use in a method for treating a subject suffering from an eye disease or disorder, the method comprising: administering to the subject one or more of the modified viral vectors packaging a recombinant viral-based genome, wherein the recombinant viral-based genome comprises a cDNA insert encoding an exogenous chemokine receptor polypeptide; wherein, in use, the one or more of the modified viral vectors infects and causes increased expression of the chemokine receptor in retinal cells of the subject; optionally, wherein the cDNA insert is operably linked to at least one promoter and/or enhancer sequence for expression of the exogenous chemokine receptor polypeptide in retinal cells of the subject.
84. The modified viral vector for use according to Clause 82 or Clause 83, wherein the viral vector is selected from the group consisting of: retroviruses (such as influenza, simian immunodeficiency virus (SIV), human immunodeficiency virus (HIV), lentivirus, and Moloney murine leukaemia); adenoviruses; adeno-associated viruses (AAV); sendai virus (SeV); herpes simplex virus (HSV); and chimeric viruses.
85. The modified viral vector for use according to any of Clauses 82 Clauseto 84, wherein the viral vector is selected from:
   (i) an adeno-associated virus (AAV);
   (ii) a sendai virus (SeV);
   (iii) an AAV vector selected from the group consisting of: AAV1, AAV2, AAV3, AAV4, AAV5, AAV6, AAV6.2, AAV7, AAV8, AAV9, rhlO, AAVrh64RI, AAVrh64R2 and rh8 or variants thereof; or
   (iv) an AAV vector selected from AAV2/1 and AAV2/9 viral subtypes.
86. An RNA molecule for use in a method of treating a subject suffering from tissue damage and/or a degenerative disease, or from an eye disease or disorder, the method comprising: administering to the subject one or more of the RNA molecules, wherein the RNA molecule encodes at least one exogenous chemokine receptor polypeptide; wherein, in use, the RNA molecule leads to expression of the chemokine receptor in target cells of the subject.
87. The RNA molecule for use according to Clause 86, wherein the RNA molecule comprises mRNA.
88. The RNA molecule for use according to Clause 86 or 87, wherein the RNA molecule is encapsulated or otherwise associated with a delivery particle, optionally wherein the delivery particle is selected from one or more of lipid nanoparticles; liposomes; nanosomes; and lipidoid nanoparticles.
89. The modified viral vector for use according to any of Clauses 82 to 85 or the RNA molecule for use according to any of Clauses 86 to 88, wherein the chemokine receptor is selected from one or more chemokine receptor selected from a group consisting of:
   (i) Ccr5, Cxcr6, Ccr1, Cxcr2 and/or Ccr3;
   (ii) Ccr5, Cxcr6, Ccr1 and/or Cxcr2; or
   (iii) Ccr5 and/or Cxcr6.
90. The modified viral vector for use according to any of Clauses 82 to 85 or 89, or the RNA molecule for use according to any of Clauses 86 to 89, wherein the exogenous chemokine receptor is the Ccr5 chemokine receptor and/or the Cxcr6 chemokine receptor.
91. The modified viral vector for use according to any of Clauses 83 to 85, 89 or 90, or the RNA molecule for use according to any of Clauses 86 to 90, wherein the eye disease or disorder is caused by a degeneration of the retina (retinal disease), such as: retinitis pigmentosa (RP), macular degeneration (MD), age related macular degeneration (AMD or ARMD), macula edema due of other reasons, retinal vessel occlusions, diabetic retinopathy, glaucoma and other optic neuropathies, etc., and conditions associated therewith; Bassen-kornzweig syndrome, choroideremia, gyrate atrophy, Refsum syndrome, Usher syndrome, color blindness, blue cone monochromacy, achromatopsia, incomplete achromatopsia, oligocone trichromacy, Stargardt's Disease, Bardet-Biedl syndrome, Bornholm eye disease, Best's Disease and Leber's congenital amaurosis.
92. The modified viral vector for use according to any of Clauses 83 to 85 or 89 to 91, or the RNA molecule for use according to any of Clauses 86 to 91, wherein the eye disease or disorder is selected from retinitis pigmentosa (RP), macular degeneration (MD), age related macular degeneration (AMD or ARMD), diabetic retinopathy, Stargardt's Disease and glaucoma.
93. The modified viral vector for use according to any of Clauses 82 to 85 or 89 to 92, or the RNA molecule for use according to any of Clauses 86 to 91, wherein the method comprises administering to the subject the viral-based vector comprising a cDNA insert encoding an exogenous chemokine receptor polypeptide, or the RNA molecule encoding at least one exogenous chemokine receptor polypeptide, in combination with administering to the subject a viral-based vector comprising a cDNA insert encoding a secretable polypeptide molecule or an RNA molecule encoding a secretable polypeptide molecule that is capable of maintaining or enhancing the activity, survival and/or number of cells in proximity to a cell in which the secretable polypeptide is expressed.
94. The modified viral vector for use according to Clause 93, or the RNA molecule for use according to Clause 93, wherein the secretable polypeptide molecule is a neuroprotective factor, an anti-angiogenic factor and/or a fragment or variant thereof; optionally, wherein the neuroprotective factor comprises a GLP-1 peptide or fragment or variant thereof, and the anti-angiogenic factor comprises endostatin, or a fragment or variant thereof.
95. The modified viral vector for use according to Clause 93 or Clause 94, or the RNA molecule for use according to Clause 93 or 94, wherein the secretable polypeptide molecule is a paracrine factor; optionally, wherein the paracrine factor is selected from VEGF, IL6, IL8, GDNF, NT3, NeuroD1 and/or MCP1.
96. The modified viral vector for use according to any of Clauses 82 to 85, or 89 to 95, or the RNA molecule for use according to any of Clauses 86 to 95, wherein the method comprises:
   (i) non-systemic administration of the modified viral vector or RNA molecule;
   (ii) systemic administration of the modified viral vector or RNA molecule; or
   (iii) intraocular administration of the modified viral vector or RNA molecule.
97. The modified viral vector for use according to Clause 96 or the RNA molecule for use according to Clause 96, wherein intraocular administration comprises:
   (i) injecting the modified viral vector or RNA molecule into the intravitreal space such that the modified viral vector infects or the RNA molecule is delivered to cells of the ganglion cell layer (GCL); or
   (ii) injecting the modified viral vector or RNA molecule into the subretinal space such that the modified viral vector infects or the RNA molecule is delivered to cells of the outer nuclear layer (ONL).
98. The modified viral vector for use according to any of Clauses 82 to 85 or 89 to 97, wherein the at least one promoter and/or enhancer sequence is:
   (i) an inducible transcriptional responsive regulatory element; optionally a drug responsive regulatory element; or
   (ii) a constitutive promoter and/or enhancer combination.
99. The modified viral vector for use according to any of Clauses 82 to 85 or 89 to 98, or the RNA molecule for use according to any of Clauses 86 to 97, wherein the method comprises contacting a stem cell with the modified viral vector or RNA molecule under conditions to allow the viral vector to infect the stem cell, or the RNA molecule to enter the stem cell, to form a genetically-modified stem cell.
100. The modified viral vector for use according to Clause 99, or the RNA molecule for use according to Clause 99, wherein the step of contacting the stem cell with the modified viral vector or RNA molecule occurs before the genetically-modified stem cell is administered to the subject; e.g. wherein the step of contacting the stem cell with the modified viral vector or RNA molecule occurs in vitro or ex vivo.
101. The modified viral vector for use according to Clause 99 or 100, or the RNA molecule for use according to Clause 99 or 100, wherein the stem cell is selected from the group consisting of: pluripotent stem cells, multipotent stem cells and unipotent stem cells.
102. The modified viral vector for use according to any of Clauses 99 to 101, or the RNA molecule for use according to any of Clauses 99 to 101, wherein the stem cell is an embryonic stem cell (ES cell), pluripotent stem cell (PS cell), induced pluripotent stem cell (iPS cell), mesenchymal stem cell (MSC), bone marrow-derived stem cell, retinal progenitor cell, photoreceptor precursor cell, or hybrid pluripotent cell (e.g. a fusion between a Muller Glia cell and a bone marrow derived stem cell, a fusion between an MSC or bone marrow derived stem cells with a neuron cell, or a fusion between an iPSC-derived progenitor cell with a Muller Glia cell).
103. The modified viral vector for use according to any of Clauses 99 to 102, or the RNA molecule for use according to any of Clauses 99 to 102, wherein the stem cell is a photoreceptor precursor cell, horizontal cell precursor, bipolar cell precursor, amacrine cell precursor, ganglion cell precursor, Muller cell precursor or retinal pigment epithelial precursor cell.
104. The modified viral vector for use according to any of Clauses 99 to 103, or the RNA molecule for use according to any of Clauses 99 to 103, wherein the stem cell is selected from the group consisting of: an induced pluripotent stem cell (iPS cell), a mesenchymal stem cell (MSC) and a photoreceptor precursor cell (such as a rod precursor cell or a cone precursor cell).
105. The modified viral vector for use according to any of Clauses 99 to 104, or the RNA molecule for use according to any of Clauses 99 to 104, wherein the stem cell is:
   (i) a mesenchymal stem cell (MSC); or
   (ii) a photoreceptor precursor cell.
106. The modified viral vector for use according to any of Clauses 99 to 105, or the RNA molecule for use according to any of Clauses 99 to 105, wherein the stem cell is selected from the group consisting of: a human stem cell, a non-human primate stem cell, a pig stem cell, a dog stem cell, a cat stem cell, or a rodent stem cell (including a mouse stem cell, a hamster stem cell and a rabbit stem cell).
107. The modified viral vector for use according to any of Clauses 99 to 106, or the RNA molecule for use according to any of Clauses 99 to 106, wherein the stem cell is a human stem cell, a non-human primate stem cell, or a mouse stem cell.
108. A modified viral vector packaging a recombinant viral-based genome, for use in a method for treating a subject suffering from tissue damage and/or a degenerative disease, or from an eye disease or disorder, the method comprising: administering to the subject one or more of the modified viral vectors packaging a recombinant viral-based genome, wherein the recombinant viral-based genome comprises a cDNA insert encoding an exogenous chemokine polypeptide; wherein, in use, the one or more of the modified viral vectors infects and causes increased expression of the chemokine in target cells of the subject; optionally, wherein the cDNA insert is operably linked to at least one promoter and/or enhancer sequence for expression of the exogenous chemokine polypeptide in target cells of the subject.
109. The modified viral vector for use according to Clause 108, wherein the viral vector is selected from the group consisting of: retroviruses (such as influenza, simian immunodeficiency virus (SIV), human immunodeficiency virus (HIV), lentivirus, and Moloney murine leukaemia); adenoviruses; adeno-associated viruses (AAV); sendai virus (SeV); herpes simplex virus (HSV); and chimeric viruses.
110. The modified viral vector for use according to Clause 108 or 109, wherein the viral vector is selected from:
   (i) an adeno-associated virus (AAV);
   (ii) a sendai virus (SeV);
   (iii) an AAV vector selected from the group consisting of: AAV1, AAV2, AAV3, AAV4, AAV5, AAV6, AAV6.2, AAV7, AAV8, AAV9, rhlO, AAVrh64RI, AAVrh64R2 and rh8 or variants thereof; or
   (iv) an AAV vector selected from AAV2/1 and AAV2/9 viral subtypes.
111. An RNA molecule for use in a method of treating a subject suffering from tissue damage and/or a degenerative disease, or from an eye disease or disorder, the method comprising: administering to the subject one or more of the RNA molecules, wherein the RNA molecule encodes at least one exogenous chemokine polypeptide; wherein, in use, the RNA molecule leads to expression of the chemokine in target cells of the subject.
112. The RNA molecule for use according to Clause 111, wherein the RNA molecule comprises mRNA.
113. The RNA molecule for use according to Clause 111 or 112, wherein the RNA molecule is encapsulated or otherwise associated with a delivery particle, optionally wherein the delivery particle is selected from one or more of lipid nanoparticles; liposomes; nanosomes; and lipidoid nanoparticles.
114. The modified viral vector for use according to any of Clauses 108 to 110, or the RNA molecule for use according to any of Clauses 111 to 113, wherein the exogenous chemokine is a suitable ligand for one or more chemokine receptor selected from a group consisting of:
   (i) Ccr5, Cxcr6, Ccr1, Cxcr2 and/or Ccr3;
   (ii) Ccr5, Cxcr6, Ccr1 and/or Cxcr2; or
   iii) Ccr5 and/or Cxcr6.
115. The modified viral vector for use according to any of Clauses 108 to 110 or 114, or the RNA molecule for use according to any of Clauses 111 to 114, wherein the exogenous chemokine is selected from the group consisting of CCL3, CCL5, CCL7, CCL23, CCL4, CCL3L1, CCL11, CCL26, CCL13, IL8, CXCL1, CXCL2, CXCL5, and/or CCL16.
116. The modified viral vector for use according to any of Clauses 108 to 110, 114 or 115, or the RNA molecule for use according to any of Clauses 111 to 114, wherein the eye disease or disorder is caused by a degeneration of the retina (retinal disease), such as: retinitis pigmentosa (RP), macular degeneration (MD), age related macular degeneration (AMD or ARMD), macula edema due of other reasons, retinal vessel occlusions, diabetic retinopathy, glaucoma and other optic neuropathies, etc., and conditions associated therewith; Bassen-kornzweig syndrome, choroideremia, gyrate atrophy, Refsum syndrome, Usher syndrome, color blindness, blue cone monochromacy, achromatopsia, incomplete achromatopsia, oligocone trichromacy, Stargardt's Disease, Bardet-Biedl syndrome, Bornholm eye disease, Best's Disease and Leber's congenital amaurosis.
117. The modified viral vector for use according to any of Clauses 108 to 110 or 114 to 116, or the RNA molecule for use according to any of Clauses 111 to 116, wherein the eye disease or disorder is selected from retinitis pigmentosa (RP), macular degeneration (MD), age related macular degeneration (AMD or ARMD), diabetic retinopathy, Stargardt's Disease and glaucoma.
118. The modified viral vector for use according to any of Clauses 108 to 110 or 114 to 117, or the, or the RNA molecule for use according to any of Clauses 111 to 117, wherein the method comprises administering to the subject the viral-based vector comprising a cDNA insert encoding an exogenous chemokine polypeptide, or the RNA molecule encoding at least one exogenous chemokine polypeptide, in combination with administering to the subject a viral-based vector comprising a cDNA insert encoding a secretable polypeptide molecule or an RNA molecule encoding a secretable polypeptide molecule that is capable of maintaining or enhancing the activity, survival and/or number of cells in proximity to a cell in which the secretable polypeptide is expressed.
119. The modified viral vector for use according to Clause 118, or the RNA molecule for use according to Clause 118, wherein the secretable polypeptide molecule is a neuroprotective factor, an anti-angiogenic factor and/or a fragment or variant thereof; optionally, wherein the neuroprotective factor comprises a GLP-1 peptide or fragment or variant thereof, and the anti-angiogenic factor comprises endostatin, or a fragment or variant thereof.
120. The modified viral vector for use according to Clause 118 or 119, or the RNA molecule for use according to Clause 118 or 119, wherein the secretable polypeptide molecule is a paracrine factor; optionally, wherein the paracrine factor is selected from VEGF, IL6, IL8, GDNF, NT3, NeuroD1 and/or MCP1.
121. The modified viral vector for use according to any of Clauses 108 to 110 or 114 to 120, or the RNA molecule for use according to any of Clauses 111 to 120, wherein the method comprises:
   (i) non-systemic administration of the modified viral vector or RNA molecule;
   (ii) systemic administration of the modified viral vector or RNA molecule; or
   (iii) intraocular administration of the modified viral vector or RNA molecule.
122. The modified viral vector for use according to Clause 121 or the RNA molecule for use according to Clause 121, wherein intraocular administration comprises:
   (i) injecting the modified viral vector or RNA molecule into the intravitreal space such that the modified viral vector infects or the RNA molecule is delivered to cells of the ganglion cell layer (GCL); or
   (ii) injecting the modified viral vector or RNA molecule into the subretinal space such that the modified viral vector infects or the RNA molecule is delivered to cells of the outer nuclear layer (ONL).
123. The modified viral vector for use according to any of Clauses 108 to 110 or 114 to 122, wherein the at least one promoter and/or enhancer sequence is:
   (i) an inducible transcriptional responsive regulatory element; optionally a drug responsive regulatory element; or
   (ii) a constitutive promoter and/or enhancer combination.
124. The modified viral vector for use according to any of Clauses 108 to 110 or 114 to 123, or the RNA molecule for use according to any of Clauses 111 to 122, wherein the method further comprises administering the genetically-modified stem cell according to any of Clauses 1 to 35, the pharmaceutical composition of any of Clauses 36 to 41, or the genetically modified cell for use according to any of Clauses 42 to 64 to the subject.
125. The modified viral vector for use according to Clause 124, or the RNA molecule for use according to Clause 124, wherein the genetically-modified stem cell, the pharmaceutical composition, or the genetically modified cell for use is administered to the subject concurrently with, subsequent to, or consecutively with the modified viral vector or RNA molecule.
126. The modified viral vector for use according to Clause 124 or 125, or the RNA molecule for use according to Clause 124 or 125, wherein the exogenous chemokine encoded by the modified viral vector or RNA molecule is a suitable ligand for the exogenous chemokine receptor comprised by the genetically-modified stem cell.
127. A pharmaceutical composition comprising the modified viral vector for use according to any of Clauses 82 to 85 or 89 to 110 or 114 to 126, or the RNA molecule for use according to any of Clauses 86 to 107 or 111 to 122 or 124 to 126, and a pharmaceutically acceptable carrier and/or excipient.
128. The pharmaceutical composition according to Clause 108, which is formulated as an injectable composition; optionally, wherein the injectable composition is suitable for intraocular administration.
129. A method of producing a genetically-modified stem cell, comprising contacting a stem cell with one or more modified viral vectors packaging a recombinant viral-based genome, wherein the recombinant viral-based genome comprises a cDNA insert encoding an exogenous chemokine receptor polypeptide, such that the one or more modified viral vectors infects and causes increased expression of the chemokine receptor in the stem cell; optionally, wherein the cDNA insert is operably linked to at least one promoter and/or enhancer sequence for expression of the exogenous chemokine receptor polypeptide in the stem cell.
130. The method of Clause 129, wherein the viral vector is selected from the group consisting of: retroviruses (such as influenza, simian immunodeficiency virus (SIV), human immunodeficiency virus (HIV), lentivirus, and Moloney murine leukaemia); adenoviruses; adeno-associated viruses (AAV); sendai virus (SeV); herpes simplex virus (HSV); and chimeric viruses.
131. The method of Clause 129 or 130, wherein the viral vector is selected from:
   (i) an adeno-associated virus (AAV);
   (ii) a sendai virus (SeV);
   (iii) an AAV vector selected from the group consisting of: AAV1, AAV2, AAV3, AAV4, AAV5, AAV6, AAV6.2, AAV7, AAV8, AAV9, rhlO, AAVrh64RI, AAVrh64R2 and rh8 or variants thereof; or
   (iv) an AAV vector selected from AAV2/1 and AAV2/9 viral subtypes.
132. The method of any of Clauses 129 to 131, wherein the at least one promoter and/or enhancer sequence is:
   (i) an inducible transcriptional responsive regulatory element; optionally a drug responsive regulatory element; or
   (ii) a constitutive promoter and/or enhancer combination
133. A method of producing a genetically-modified stem cell, comprising contacting a stem cell with one or more RNA molecules encoding at least one exogenous chemokine receptor polypeptide; wherein, in use, the RNA molecule leads to expression of the chemokine receptor in the stem cell, optionally wherein the RNA molecule comprises mRNA.
134. The method according to Clause 134, wherein the RNA molecule is encapsulated or otherwise associated with a delivery particle, optionally wherein the delivery particle is selected from one or more of lipid nanoparticles; liposomes; nanosomes; and lipidoid nanoparticles.
135. The method according to any of Clauses 129 to 134, wherein the chemokine receptor is selected from one or more chemokine receptor selected from a group consisting of:
   (i) Ccr5, Cxcr6, Ccr1, Cxcr2 and/or Ccr3;
   (ii) Ccr5, Cxcr6, Ccr1 and/or Cxcr2; or
   (iii) Ccr5 and/or Cxcr6.
136. The method according to any of Clauses 129 to 135, wherein the stem cell is selected from the group consisting of: pluripotent stem cells, multipotent stem cells and unipotent stem cells.
137. The method according to any of Clauses 129 to 136, wherein the stem cell is an embryonic stem cell (ES cell), pluripotent stem cell (PS cell), induced pluripotent stem cell (iPS cell), mesenchymal stem cell (MSC), bone marrow-derived stem cell, retinal progenitor cell, photoreceptor precursor cell, or hybrid pluripotent cell (e.g. a fusion between a Muller Glia cell and a bone marrow derived stem cell, a fusion between an MSC or bone marrow derived stem cells with a neuron cell, or a fusion between an iPSC-derived progenitor cell with a Muller Glia cell).
138. The method according to any of Clauses 129 to 137, wherein the stem cell is a photoreceptor precursor cell, horizontal cell precursor, bipolar cell precursor, amacrine cell precursor, ganglion cell precursor, Muller cell precursor or retinal pigment epithelial precursor cell.
139. The method according to any of Clauses 129 to 138, wherein the stem cell is selected from the group consisting of: an induced pluripotent stem cell (iPS cell), a mesenchymal stem cell (MSC) and a photoreceptor precursor cell (such as a rod precursor cell or a cone precursor cell).
140. The method according to any of Clauses 129 to 139, wherein the stem cell is:
   (i) a mesenchymal stem cell (MSC); or
   (ii) a photoreceptor precursor cell.
141. The method according to any of Clauses 129 to 140, wherein the stem cell is selected from the group consisting of: a human stem cell, a non-human primate stem cell, a pig stem cell, a dog stem cell, a cat stem cell, or a rodent stem cell (including a mouse stem cell, a hamster stem cell and a rabbit stem cell).
142. The method according to any of Clauses 129 to 141, wherein the stem cell is a human stem cell, a non-human primate stem cell, or a mouse stem cell.

In any of the above aspect, embodiments and Clauses, and in the below claims (as appropriate), it is envisaged that the stem cell may be replaced with a somatic cell. For example, a somatic cell selected from a photoreceptor cell, horizontal cell, bipolar cell, amacrine cell, ganglion cell, Muller cell or retinal pigment epithelial cell, which is genetically modified to comprise an exogenous nucleic acid encoding a chemokine receptor, wherein the exogenous nucleic acid is operably linked to at least one promoter and/or enhancer sequence for expression of the chemokine receptor in the genetically-modified stem cell or somatic cell. It should also be appreciated that the pluripotent cell disclosed herein may be a fusion cell, for example, comprising a fusion between an MSC and a somatic cell, wherein the resultant fusion is pluripotent. Suitably, the somatic cell is a cell of the eye; particularly a retinal cell, such as a photoreceptor, horizontal cell, bipolar cell, amacrine cell, ganglion cell, retinal pigment epithelial cell or Muller cell. A particularly suitable cell fusion comprises an MSC—Muller Glia cell fusion.

Thus, although specific embodiments have been described, it would be apparent to the skilled person that modifications and variations are possible without departing from the spirit and scope of the invention, which is defined by the appended claims. As such, the appended claims intend to cover any such variations. Further, it would be apparent to the skilled person that many features described in relation to particular embodiments are combinable and envisaged for combination with features described in relation to other embodiments.

1. A genetically-modified stem cell comprising an exogenous nucleic acid encoding a chemokine receptor, wherein the exogenous nucleic acid is operably linked to at least one promoter and/or enhancer sequence for expression of the chemokine receptor in the genetically-modified stem cell.

2. The genetically modified stem cell of claim 1, which comprises one or more exogenous nucleic acid, each of the one or more exogenous nucleic acids encoding a chemokine receptor selected from the group consisting of:
(i) Ccr5, Cxcr6, Ccr1, Cxcr2, and/or Ccr3;
(ii) Ccr5, Cxcr6, Ccr1, and/or Cxcr2; or
(iii) Ccr5 and/or Cxcr6;
wherein the exogenous nucleic acid encodes a human chemokine receptor sequence, or a mouse chemokine receptor sequence.

3. The genetically-modified stem cell of claim 1, wherein the one or more exogenous nucleic acids comprises a sequence that is at least 80%, 85%, 90%, 95%, 98%, 99%, or 100% similar or identical to any one or more of SEQ ID NOs 87 to 116.

4. The genetically-modified stem cell of claim 1, wherein the stem cell is selected from the group consisting of: pluripotent stem cells, multipotent stem cells, and unipotent stem cells.

5. The genetically-modified stem cell of claim 1, wherein the stem cell is:
(i) a mesenchymal stem cell (MSC);
(ii) a photoreceptor precursor cell, horizontal cell precursor, biopolar cell precursor, amacrine cell precursor, Muller cell precursor, ganglion cell precursor, or retinal pigment epithelial precursor cell; or
(iii) a hybrid pluripotent cell.

6. The genetically-modified stem cell of claim 1, wherein the exogenous nucleic acid is:
(i) integrated into the genome of the genetically-modified stem cell; or
(ii) extrachromosomal or episomal.

7. The genetically-modified stem cell of claim 1, wherein the exogenous nucleic acid is part of a proviral sequence, wherein the proviral sequence is selected from the group consisting of: retroviruses; adenoviruses; adeno-associated viruses (AAV); sendai virus (SeV); herpes simplex virus (HSV); and chimeric viruses.

8. (canceled)

9. The genetically-modified stem cell of claim 1, wherein the exogenous nucleic acid is RNA.

10. The genetically-modified stem cell of claim 1, which is capable of secreting one or more paracrine factors selected from VEGF, IL6, IL8, GDNF, NT3, and/or MCP1.

11. The genetically-modified stem cell of claim 1, further comprising an exogenous nucleic acid encoding a selectable or screenable marker which is operably linked to at least one promoter and/or enhancer sequence for expression of the selectable or screenable marker in the genetically-modified stem cell.

12. A pharmaceutical composition comprising the genetically-modified stem cell according to any claim 1.

13. The pharmaceutical composition of claim 12, wherein the composition is formulated for intraocular administration.

14. A method for the treatment of tissue damage, degenerative disease, and/or an eye disease or disorder in a subject in need thereof, the method comprising administering to the subject the pharmaceutical composition according to claim 12.

15. (canceled)

16. The method according to claim 14, wherein the eye disease or disorder is selected from the group consisting of retinitis pigmentosa (RP), macular degeneration (MD), age related macular degeneration (AMD or ARMD), macula edema due of other reasons, retinal vessel occlusions, diabetic retinopathy, glaucoma, Bassen-kornzweig syndrome, choroideremia, gyrate atrophy, Refsum syndrome, Usher syndrome, color blindness, blue cone monochromacy, achromatopsia, incomplete achromatopsia, oligocone trichromacy, Stargardt's Disease, Bardet-Biedl syndrome, Bomholm eye disease, Best's Disease, and Leber's congenital amaurosis.

17. The method according to claim 16, wherein the method comprises intraocular injection of the genetically-modified pharmaceutical composition into the intravitreal space or into the subretinal space.

18. A method for treating a subject suffering from an eye disease or disorder, the method comprising: administering to the subject one or more the modified viral vectors packaging a recombinant viral-based genome, wherein the recombinant viral-based genome comprises a cDNA insert encoding an exogenous chemokine receptor polypeptide; wherein, the one or more modified viral vectors infects and causes increased expression of the chemokine receptor in retinal cells of the subject; and wherein the cDNA insert is operably linked to at least one promoter and/or enhancer sequence for expression of the exogenous chemokine receptor polypeptide in retinal cells of the subject.

19-23. (canceled)

24. A method of producing a genetically-modified stem cell, comprising contacting a stem cell with one or more modified viral vectors packaging a recombinant viral-based genome, wherein the recombinant viral-based genome comprises a cDNA insert encoding an exogenous chemokine receptor polypeptide, such that the one or more modified viral vectors infects and causes increased expression of the chemokine receptor in the stem cell.

25. (canceled)

26. The genetically-modified stem cell according to claim 5, wherein the hybrid pluripotent cell is a product of a fusion between a Muller Glia cell and a bone marrow derived stem cell, a fusion between an MSC or bone marrow derived stem cells with a neuron cell, or a fusion between an iPSC-derived progenitor cell with a Muller Glia cell.

27. The method according to claim 17, wherein the genetically-modified cell is an MSC, and wherein the method comprises intraocular injection of the pharmaceutical composition comprising the genetically-modified MSC into the intravitreal space such that the genetically-modified MSC integrates into and/or cooperates with the ganglion cell layer (GCL) and/or the inner nuclear layer (INL) to repair and/or protect and/or increase the thickness of the GCL and/or INL.

28. The method according to claim 17, wherein the genetically-modified cell is a photoreceptor precursor cell, and wherein the method comprises intraocular injection of the pharmaceutical composition comprising the photoreceptor precursor cell into the subretinal space such that the photoreceptor precursor cell integrates into the outer nuclear layer (ONL) and/or cooperates with photoreceptor cells of the outer nuclear layer (ONL) to repair and/or protect and/or increase the thickness of the ONL.


SEQUENCE LISTING (116 sequences)

SEQ ID NO: 1 - DNA, 23 bp, Synthetic Sequence
  mIl1-beta FW primer
gccaccttttgacagtgatgaga

SEQ ID NO: 2 - DNA, 20 bp, Synthetic Sequence
  mIl1-beta RV primer
ggacagcccaggtcaaaggt

SEQ ID NO: 3 - DNA, 21 bp, Synthetic Sequence
  mCcl5 FW primer
tgcagaggactctgagacagc

SEQ ID NO: 4 - DNA, 19 bp, Synthetic Sequence
  mCcl5 RV primer
gagtggtgtccgagccata

SEQ ID NO: 5 - DNA, 21 bp, Synthetic Sequence
  mCcl6 FW primer
tctttatccttgtggctgtcc

SEQ ID NO: 6 - DNA, 20 bp, Synthetic Sequence
  mCcl6 RV primer
tggagggttatagcgacgat

SEQ ID NO: 7 - DNA, 20 bp, Synthetic Sequence
  mCcl12 FW primer
ggtattggctggaccagatg

SEQ ID NO: 8 - DNA, 18 bp, Synthetic Sequence
  mCcl12 RV primer
gggacactggctgcttgt

SEQ ID NO: 9 - DNA, 20 bp, Synthetic Sequence
  mCxcl1 FW primer
agactccagccacactccaa

SEQ ID NO: 10 - DNA, 18 bp, Synthetic Sequence
  mCxcl1 RV primer
tgacagcgcagctcattg

SEQ ID NO: 11 - DNA, 20 bp, Synthetic Sequence
  mCxcl2 FW primer
actccagactccagccacac

SEQ ID NO: 12 - DNA, 20 bp, Synthetic Sequence
  mCxcl2 RV primer
cagttcactggccacaacag

SEQ ID NO: 13 - DNA, 24 bp, Synthetic Sequence
  mCxcl5 FW primer
tcttgggtgtgttaagagtgttct

SEQ ID NO: 14 - DNA, 24 bp, Synthetic Sequence
  mCxcl5 RV primer
cacagcagctttctaaaaccataa

SEQ ID NO: 15 - DNA, 20 bp, Synthetic Sequence
  mCxcl9 FW primer
ccatgaagtccgctgttctt

SEQ ID NO: 16 - DNA, 21 bp, Synthetic Sequence
  mCxcl9 RV primer
tgagggatttgtagtggatcg

SEQ ID NO: 17 - DNA, 20 bp, Synthetic Sequence
  mCxcl10 FW primer
atcagcaccatgaacccaag

SEQ ID NO: 18 - DNA, 20 bp, Synthetic Sequence
  mCxcl10 RV primer
ttccctatggccctcattct

SEQ ID NO: 19 - DNA, 18 bp, Synthetic Sequence
  mCxcl11 FW primer
gcggctgctgagatgaac

SEQ ID NO: 20 - DNA, 20 bp, Synthetic Sequence
  mCxcl11 RV primer
cgcccctgtttgaacataag

SEQ ID NO: 21 - DNA, 23 bp, Synthetic Sequence
  mCxcl16 FW primer
tgaactagtggactgctttgagc

SEQ ID NO: 22 - DNA, 20 bp, Synthetic Sequence
  mCxcl16 RV primer
gcaaatgtttttggtggtga

SEQ ID NO: 23 - DNA, 20 bp, Synthetic Sequence
  hIL1-beta FW primer
tacctgtcctgcgtgttgaa

SEQ ID NO: 24 - DNA, 23 bp, Synthetic Sequence
  hIL1-beta RV primer
tctttgggtaatttttgggatct

SEQ ID NO: 25 - DNA, 21 bp, Synthetic Sequence
  hTNF-alpha FW primer
cagcctcttctccttcctgat

SEQ ID NO: 26 - DNA, 20 bp, Synthetic Sequence
  hTNF-alpha RV primer
gccagagggctgattagaga

SEQ ID NO: 27 - DNA, 18 bp, Synthetic Sequence
  hCCL2 FW primer
agtctctgccgcccttct

SEQ ID NO: 28 - DNA, 19 bp, Synthetic Sequence
  hCCL2 RV primer
gtgactggggcattgattg

SEQ ID NO: 29 - DNA, 20 bp, Synthetic Sequence
  hCCL5 FW primer
cgctgtcatcctcattgcta

SEQ ID NO: 30 - DNA, 20 bp, Synthetic Sequence
  hCCL5 RV primer
ggtgtggtgtccgaggaata

SEQ ID NO: 31 - DNA, 20 bp, Synthetic Sequence
  hCCL21 FW primer
tccatcccagctatcctgtt

SEQ ID NO: 32 - DNA, 19 bp, Synthetic Sequence
  hCCL21 RV primer
agctcctttgggtctgcac

SEQ ID NO: 33 - DNA, 22 bp, Synthetic Sequence
  hCCL22 FW primer
cgtggtgaaacacttctactgg

SEQ ID NO: 34 - DNA, 22 bp, Synthetic Sequence
  hCCL22 RV primer
ccttatccctgaaggttagcaa

SEQ ID NO: 35 - DNA, 20 bp, Synthetic Sequence
  hCCL23 FW primer
ccaggaggatgaaggtctcc

SEQ ID NO: 36 - DNA, 23 bp, Synthetic Sequence
  hCCL23 RV primer
catcatgaactctgtctctgcat

SEQ ID NO: 37 - DNA, 20 bp, Synthetic Sequence
  hCXCL1 FW primer
tcctgcatcccccatagtta

SEQ ID NO: 38 - DNA, 20 bp, Synthetic Sequence
  hCXCL1 RV primer
cttcaggaacagccaccagt

SEQ ID NO: 39 - DNA, 22 bp, Synthetic Sequence
  hCXCL2 FW primer
cccatggttaagaaaatcatcg

SEQ ID NO: 40 - DNA, 20 bp, Synthetic Sequence
  hCXCL2 RV primer
cttcaggaacagccaccaat

SEQ ID NO: 41 - DNA, 26 bp, Synthetic Sequence
  hCXCL3 FW primer
aaatcatcgaaaagatactgaacaag

SEQ ID NO: 42 - DNA, 18 bp, Synthetic Sequence
  hCXCL3 RV primer
ggtaagggcagggaccac

SEQ ID NO: 43 - DNA, 18 bp, Synthetic Sequence
  hCXCL6 FW primer
gtccttcgggctccttgt

SEQ ID NO: 44 - DNA, 20 bp, Synthetic Sequence
  hCXCL6 RV primer
cagcacagcagagacaggac

SEQ ID NO: 45 - DNA, 20 bp, Synthetic Sequence
  hCXCL9 FW primer
tgttcccctttgcttcattc

SEQ ID NO: 46 - DNA, 19 bp, Synthetic Sequence
  hCXCL9 RV primer
gaaaggcactgcattgtgg

SEQ ID NO: 47 - DNA, 23 bp, Synthetic Sequence
  hCXCL10 FW primer
gaaagcagttagcaaggaaaggt

SEQ ID NO: 48 - DNA, 26 bp, Synthetic Sequence
  hCXCL10 RV primer
gacatatactccatgtagggaagtga

SEQ ID NO: 49 - DNA, 19 bp, Synthetic Sequence
  hCXCL11 FW primer
agtgtgaagggcatggcta

SEQ ID NO: 50 - DNA, 20 bp, Synthetic Sequence
  hCXCL11 RV primer
tcttttgaacatggggaagc

SEQ ID NO: 51 - DNA, 20 bp, Synthetic Sequence
  hCXCL16 FW primer
gccctttcctatgtgctgtg

SEQ ID NO: 52 - DNA, 24 bp, Synthetic Sequence
  hCXCL16 RV primer
caggtatataatgaaccggcagat

SEQ ID NO: 53 - DNA, 23 bp, Synthetic Sequence
  mCcr1 FW primer
ctgtgtggacaaaatactctgga

SEQ ID NO: 54 - DNA, 20 bp, Synthetic Sequence
  mCcr1 RV primer
tggggtaggcttctgtgaaa

SEQ ID NO: 55 - DNA, 20 bp, Synthetic Sequence
  mCcr3 FW primer
gaatcaaagagctggggtca

SEQ ID NO: 56 - DNA, 18 bp, Synthetic Sequence
  mCcr3 RV primer
caggaggccgatgatgaa

SEQ ID NO: 57 - DNA, 23 bp, Synthetic Sequence
  mCcr5 FW primer
caactttggggtgataacaagtg

SEQ ID NO: 58 - DNA, 25 bp, Synthetic Sequence
  mCcr5 RV primer
tggtaaagattatttctgggagaga

SEQ ID NO: 59 - DNA, 20 bp, Synthetic Sequence
  mCxcr2 FW primer
caggaccaggaatgggagta

SEQ ID NO: 60 - DNA, 20 bp, Synthetic Sequence
  mCxcr2 RV primer
tcccctccaaatatccccta

SEQ ID NO: 61 - DNA, 20 bp, Synthetic Sequence
  mCxcr3 FW primer
gtggccaagtcagtcacctc

SEQ ID NO: 62 - DNA, 20 bp, Synthetic Sequence
  mCxcr3 RV primer
cccacaaaggcatagagcag

SEQ ID NO: 63 - DNA, 21 bp, Synthetic Sequence
  mCxcr6 FW primer
ccagctttaagtatgccatcg

SEQ ID NO: 64 - DNA, 20 bp, Synthetic Sequence
  mCxcr6 RV primer
ttaaggcaagcccgaaagta

SEQ ID NO: 65 - DNA, 19 bp, Synthetic Sequence
  mCyclinD1 FW primer
gagattgtgccatccatgc

SEQ ID NO: 66 - DNA, 21 bp, Synthetic Sequence
  mCyclinD1 RV primer
ctcctccttcgcacttctgct

SEQ ID NO: 67 - DNA, 26 bp, Synthetic Sequence
  mBDNF FW primer
cagtggacatgtccggtgggacggtc

SEQ ID NO: 68 - DNA, 27 bp, Synthetic Sequence
  mBDNF RV primer
ttcttggcaacggcaacaaaccacaac

SEQ ID NO: 69 - DNA, 20 bp, Synthetic Sequence
  mNGF FW primer
tatactggccgcagtgaggt

SEQ ID NO: 70 - DNA, 22 bp, Synthetic Sequence
  mNGF RV primer
ggacattgctatctgtgtacgg

SEQ ID NO: 71 - DNA, 18 bp, Synthetic Sequence
  mPDGFa FW primer
gatgaggacctgggcttg

SEQ ID NO: 72 - DNA, 20 bp, Synthetic Sequence
  mPDGFa RV primer
gatcaactcccggggtatct

SEQ ID NO: 73 - DNA, 24 bp, Synthetic Sequence
  mCTNF FW primer
gacctgactgctcttatggaatct

SEQ ID NO: 74 - DNA, 19 bp, Synthetic Sequence
  mCTNF RV primer
gcctggaggttctcttgga

SEQ ID NO: 75 - DNA, 25 bp, Synthetic Sequence
  mCcr1-CDS FW primer
atggagatttcagatttcacagaag

SEQ ID NO: 76 - DNA, 19 bp, Synthetic Sequence
  mCcr1-CDS RV primer
tcagaagccagcagagagc

SEQ ID NO: 77 - DNA, 24 bp, Synthetic Sequence
  mCcr3-CDS FW primer
atggcattcaacacagatgaaatc

SEQ ID NO: 78 - DNA, 24 bp, Synthetic Sequence
  mCcr3-CDS RV primer
ctaaaacaccacagagatttcttg

SEQ ID NO: 79 - DNA, 23 bp, Synthetic Sequence
  mCcr5-CDS FW primer
atggattttcaagggtcagttcc

SEQ ID NO: 80 - DNA, 24 bp, Synthetic Sequence
  mCcr5-CDS RV primer
tcataaaccagtagaaacttcatg

SEQ ID NO: 81 - DNA, 24 bp, Synthetic Sequence
  mCxcr2-CDS FW primer
atgggagaattcaaggtggataag

SEQ ID NO: 82 - DNA, 23 bp, Synthetic Sequence
  mCxcr2-CDS RV primer
ttagagggtagtagaggtgtttg

SEQ ID NO: 83 - DNA, 24 bp, Synthetic Sequence
  mCxcr3-CDS FW primer
atgtaccttgaggttagtgaacgt

SEQ ID NO: 84 - DNA, 20 bp, Synthetic Sequence
  mCxcr3-CDS RV primer
ttacaagcccaggtaggagg

SEQ ID NO: 85 - DNA, 23 bp, Synthetic Sequence
  mCxcr6-CDS FW primer
atggatgatgggcatcaagagtc

SEQ ID NO: 86 - DNA, 25 bp, Synthetic Sequence
  mCxcr6-CDS RV primer
ctacaattggaacatactggtggtc

SEQ ID NO: 87 - DNA, 2646 bp, Homo sapiens
  human CCR1
agacttcacggacaaagtcccttggaaccagagagaagccgggatggaaactccaaacaccacagaggactatgacacgaccacagagtttgactatggggatgcaactccgtgccagaaggtgaacgagagggcctttggggcccaactgctgccccctctgtactccttggtatttgtcattggcctggttggaaacatcctggtggtcctggtccttgtgcaatacaagaggctaaaaaacatgaccagcatctacctcctgaacctggccatttctgacctgctcttcctgttcacgcttcccttctggatcgactacaagttgaaggatgactgggtttttggtgatgccatgtgtaagatcctctctgggttttattacacaggcttgtacagcgagatctttttcatcatcctgctgacgattgacaggtacctggccatcgtccacgccgtgtttgccttgcgggcacggaccgtcacttttggtgtcatcaccagcatcatcatttgggccctggccatcttggcttccatgccaggcttatacttttccaagacccaatgggaattcactcaccacacctgcagccttcactttcctcacgaaagcctacgagagtggaagctgtttcaggctctgaaactgaacctctttgggctggtattgcctttgttggtcatgatcatctgctacacagggattataaagattctgctaagacgaccaaatgagaagaaatccaaagctgtccgtttgatttttgtcatcatgatcatcttttttctcttttggaccccctacaatttgactatacttatttctgttttccaagacttcctgttcacccatgagtgtgagcagagcagacatttggacctggctgtgcaagtgacggaggtgatcgcctacacgcactgctgtgtcaacccagtgatctacgccttcgttggtgagaggttccggaagtacctgcggcagttgttccacaggcgtgtggctgtgcacctggttaaatggctccccttcctctccgtggacaggctggagagggtcagctccacatctccctccacaggggagcatgaactctctgctgggttctgactcagaccataggaggccaacccaaaataagcaggcgtgacctgccaggcacactgagccagcagcctggctctcccagccaggttctgactcttggcacagcatggagtcacagccacttgggatagagagggaatgtaatggtggcctggggcttctgaggcttctggggcttcagtcttttccatgaacttctcccctggtagaaagaagatgaatgagcaaaaccaaatattccagagactgggactaagtgtaccagagaagggcttggactcaagcaagatttcagatttgtgaccattagcatttgtcaacaaagtcacccacttcccactattgcttgcacaaaccaattaaacccagtagtggtgactgtgggctccattcaaagtgagctcctaagccatgggagacactgatgtatgaggaatttctgttcttccatcacctccccccccccgccaccctcccactgccaaagaacttggaaatagtgatttccacagtgactccactctgagtcccagagccaatcagtagccagcatctgcctccccttcactcccaccgcaggatttgggctcttggaatcctggggaacatagaactcatgacggaagagttgagacctaacgagaaatagaaatggggaactactgctggcagtggaactaagaaagcccttaggaagaatttttatatccactaaaatcaaacaattcagggagtgggctaagcacgggccatatgaataacatggtgtgcttcttaaaatagccataaaggggagggactcatcatttccatttacccttcttttctgactatttttcagaatctctcttcttttcaagttgggtgatatgttggtagattctaatggctttattgcagcgattaataacaggcaaaaggaagcagggttggtttcccttctttttgttcttcatctaagccttctggttttatgggtcagagttccgactgccatcttggacttgtcagcaaaaaaaaaaaataataataataataaggcctgctgtgtaagctgacagtatttgtagctgatagggggttgggaggaaagtgtctactaggagggtggggtgagattctgtgttgatgtaggaggccgagaaggcccttaactcaaagtagcttatttatccaaaatgttctggatgcatcatctccaaccaaggaccccttatttatcatgcctttgttctcttttccctcagatgtatatttctttaaaaataattttcctaataacaaaacttatttctaaaacagcttaaaaattcaaagaaaaaccccaaacactgacattacctacacttccactacccaaagacaaaatgtgcccactgtgtgcttttgagtgtattttcttttagtttgttttttgttgggtgcatatttatgataataacaatgatggacttcaattgtactcactgttctattgttggttttaattagcagcaagttgtgatcactttcccaggtgaataaatcatttcaaagca

SEQ ID NO: 88 - DNA, 2790 bp, Homo sapiens
  human CCR3 X2
gaccctggccgtcatcaggttcaacctcatattcaactgctgggccttgtcttctggccacacactctctccaaatcagcgatggtggggaactgaggagccagaggccattgtggaagaactccaagaaaacaatctccaggttatcacatctaaagcctgagcaagatttgaggctgggaagaggtggatcccagaattggaagaaggagtggtgacgcctaagctatcactggacatatcaaggacttcactaaattagcagctgctatggattggattatgctattatgaataagaatgctgatgggagcacacacaaaccatttgttcctcagtccattttcctcctcaaaagcctggaatgtgccattgatcagtgggagatgtacctggacagacccatgaaaagagatcaacaagttccacccaagggaccctatttttcctaatttcatttgaaatggcttctaattgtccttctttcattcctgcttcctaccagttttacagctttttctggtttcaaatgtgaactcacatacactctcatttttcctcatcacaaccccaagtgacccaatggtcctcactttcgatataagtaaaggaggctctgcattaagggcttgtccaaggcacgcagctgagaggcgctaggactggctccatttccatctctattctcactgactttgactacccagaaccccaacatgtggggcctcagtattcgatcaattattctattaagaagcaaaaacaattccccgcattggccccagttattaagcatttctcagatttaccttgagaaatgcccatcggcctgtatattcacatcttcacccttgtcccttcctcctagaaaggagaaagtcagttggatgccctctgaggaactagtgcatggcttaactgtccttccatgactcctgccttatctgttttctattttcctccttttccaccgaagtctataatctcaagaaaagcaggcactggccttagggctcctggcctaagaaatatcaagtccagtgagaaatcccattgactgacccctcctgcttacccctttgtgatggagaagctcccaggggtttgctttttgcatgttaccaggcctaactcagcatcaccaggggcaagaaaaggaaagtaacctaaactaatgctgcttataattgtaattattgtaatagttaattactgtgattgtacatgtgtaacagacaaaatgtgtatttttttcacagctgctgtggattggattatgccatttggaataagaatgctgttaagagcacacaagccaggttcctcaaggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaata

SEQ ID NO: 89 - DNA, 1726 bp, Homo sapiens
  human CCR3 X4
accctggccgtcatcaggttcaacctcatattcaactgctgggccttgtcttctggccacacactctctccaaatcagcgatggtggggaactgaggagccagaggccattgtggaagaactccaagaaaacaatctccaggttatcacatctaaagcctgagcaagatttgaggctgggaagaggtggatcccagaattggaagaaggagtggtgacgcctaagctatcactggacatatcaaggacttcactaaattagcagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaata

SEQ ID NO: 90 - DNA, 1817 bp, Homo sapiens
  human CCR3 X3
ccctggccgtcatcaggttcaacctcatattcaactgctgggccttgtcttctggccacacactctctccaaatcagcgatggtggggaactgaggagccagaggccattgtggaagaactccaagaaaacaatctccaggttatcacatctaaagcctgagcaagatttgaggctgggaagaggtggatcccagaattggaagaaggagtggtgacgcctaagctatcactggacatatcaaggacttcactaaattagcaggtaccactggtcttcttgtgcttatccgggcaagaacttatcgaaatacaatagaagtttttacttagaagagattttcagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaa

SEQ ID NO: 91 - DNA, 1786 bp, Homo sapiens
  human CCR3 transcript 3
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagctgctgtggattggattatgccatttggaataagaatgctgttaagagcacacaagccaggttcctcaaggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 92 - DNA, 2609 bp, Homo sapiens
  human CCR3 X1
caggtaccactggtcttcttgtgcttatccgggcaagaacttatcgaaatacaatagaagtttttacttagaagagattttcagctgctatggattggattatgctattatgaataagaatgctgatgggagcacacacaaaccatttgttcctcagtccattttcctcctcaaaagcctggaatgtgccattgatcagtgggagatgtacctggacagacccatgaaaagagatcaacaagttccacccaagggaccctatttttcctaatttcatttgaaatggcttctaattgtccttctttcattcctgcttcctaccagttttacagctttttctggtttcaaatgtgaactcacatacactctcatttttcctcatcacaaccccaagtgacccaatggtcctcactttcgatataagtaaaggaggctctgcattaagggcttgtccaaggcacgcagctgagaggcgctaggactggctccatttccatctctattctcactgactttgactacccagaaccccaacatgtggggcctcagtattcgatcaattattctattaagaagcaaaaacaattccccgcattggccccagttattaagcatttctcagatttaccttgagaaatgcccatcggcctgtatattcacatcttcacccttgtcccttcctcctagaaaggagaaagtcagttggatgccctctgaggaactagtgcatggcttaactgtccttccatgactcctgccttatctgttttctattttcctccttttccaccgaagtctataatctcaagaaaagcaggcactggccttagggctcctggcctaagaaatatcaagtccagtgagaaatcccattgactgacccctcctgcttacccctttgtgatggagaagctcccaggggtttgctttttgcatgttaccaggcctaactcagcatcaccaggggcaagaaaaggaaagtaacctaaactaatgctgcttataattgtaattattgtaatagttaattactgtgattgtacatgtgtaacagacaaaatgtgtatttttttcacagctgctgtggattggattatgccatttggaataagaatgctgttaagagcacacaagccaggttcctcaaggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaata

SEQ ID NO: 93 - DNA, 1717 bp, Homo sapiens
  human CCR3 transcript 1
accactggtcttcttgtgcttatccgggcaagaacttatcgaaatacaatagaagtttttacttagaagagattttcagcagatgagaagctggtaacagagaccaaaatagtttggagactaaagaatcattgcacatttcactgctgagttgtattggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 94 - DNA, 1698 bp, Homo sapiens
  human CCR3 transcript 4
accactggtcttcttgtgcttatccgggcaagaacttatcgaaatacaatagaagtttttacttagaagagattttcagctgctgtggattggattatgccatttggaataagaatgctgttaagagcacacaagccagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 95 - DNA, 1638 bp, Homo sapiens
  human CCR3 transcript 2
accactggtcttcttgtgcttatccgggcaagaacttatcgaaatacaatagaagtttttacttagaagagattttcagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 96 - DNA, 3661 bp, Homo sapiens
  human CCR5 transcript variant A
cttcagatagattatatctggagtgaagaatcctgccacctatgtatctggcatagtattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataa

SEQ ID NO: 97 - DNA, 3426 bp, Homo sapiens
  human CCR5 transcript variant B
cttcagatagattatatctggagtgaagaatcctgccacctatgtatctggcatagtctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataa

SEQ ID NO: 98 - DNA, 3358 bp, Homo sapiens
  human CCR5 transcript variant C
agaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataa

SEQ ID NO: 99 - DNA, 2656 bp, Homo sapiens
  human CXCR2 transcript 2 NM_001168298.2
agtggtgatagctgagaatatgcagccgttttctccttcctgggtacagtgctattctgcctagagctctgactaccacccaaccttgaggcacagtgaagacatcggtggccactccaataacagcaggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgttta

SEQ ID NO: 100 - DNA, 2622 bp, Homo sapiens
  human CXCR2 transcript X2 XM_017003990.1
gcagtgtctttgttgagcagcagaggggagctctgactaccacccaaccttgaggcacagtgaagacatcggtggccactccaataacagcaggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttata

SEQ ID NO: 101 - DNA, 2642 bp, Homo sapiens
  human CXCR2 transcript X1 XM_005246530.3
aaaacattcagagacagaaggtggatagacaaatctccaccttcagactgctctgactaccacccaaccttgaggcacagtgaagacatcggtggccactccaataacagcaggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttata

SEQ ID NO: 102 - DNA, 2803 bp, Homo sapiens
  human CXCR2 transcript X4 XM_017003992.1
agagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttata

SEQ ID NO: 103 - DNA, 2853 bp, Homo sapiens
  human CXCR2 transcript 1 NM_001557.4
agagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgttta

SEQ ID NO: 104 - DNA, 2533 bp, Homo sapiens
  human CXCR2 transcript X3 XM_017003991.1
ataggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttata

SEQ ID NO: 105 - DNA, 2323 bp, Homo sapiens
  human CXCR6 transcript 3 NM_001386436.1
cttcttccgctggaggtagtcaacccctgtacgcggtttccttctcctgctctcctgcttatagaacaattgttcctccagaacaattcctctcagccatagtggctctgctcccaccagatgatctctcagctcatcctggtgacttgcaggggtacaggtctgccggctatgttgcccaggctggcctcaaactcctggcttcaatgatcctctcatcttagcctcttgagtactgggactataagtgcccactgccacgcctggctggttaggctacttttctgtggatcacagcccatcatccaccaatgccttgccaacaactgtaaatgtctcatccctgaatcctggaactgataacagctctgccaggtcttcaggtaaggagagcaggccaacagtctgacttccaagataaatctgcagcaggtgttcatcagaacagacaccatggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttataggccttgccagggtttcgagaagctgctctggaatttgcaagtcatggctgtgccctcttgatgtggtgaggcaggctttgtttatagcttgcgcattctcatggagaagttatcagacactctggctggtttggaatgcttcttctcaggcatgaacatgtactgttctcttcttgaacactcatgctgaaagcccaagtagggggtctaaaatttttaaggactttccttcctccatctccaagaatgctgaaaccaagggggatgacatgtgactcctatgatctcaggttctccttgattgggactggggctgaaggttgaagaggtgagcacggccaacaaagctgttgatggtaggtggcacactgggtgcccaagctcagaaggctcttctgactactgggcaaagagtgtagatcagagcagcagtgaaaacaagtgctggcaccaccaggcacctcacagaaatgagatcaggctctgcctcaccttggggcttgacttttgtataggtagatgttcagattgctttgattaatccagaataactagcaccagggactatgaatgggcaaaactgaattataagaggctgataattccagtggtccatggaatgcttgaaaaatgtgcaaaacagcgtttaagactgtaatgaatctaagcagcatttctgaagtggactctttggtggctttgcattttaaaaatgaaattttccaatgtctgccacacaaacgtatgtaaatgtatatacccacacacatacacacatatgtcatatattactagcatatgagtttcatagctaagaaataaaactgttaaagtctccaaa

SEQ ID NO: 106 - DNA, 2274 bp, Homo sapiens
  human CXCR6 transcript 4 NM_001386437.1
cttcttccgctggaggtagtcaacccctgtacgcggtttccttctcctgctctcctgcttatagaacaattgttcctccagaacaattcctctcagccatagtggctctgctcccaccagatgatctctcagctcatcctggtgacttgcaggggtacaggtctgccggctatgttgcccaggctggcctcaaactcctggcttcaatgatcctctcatcttagcctcttgagtactgggactataagtgcccactgccacgcctggctggttaggctacttttctgtggatcacagcccatcatccaccaatgccttgccaacaactgtaaatgtctcatccctgaatcctggaactgataacagctctgccaggtcttcaggtgttcatcagaacagacaccatggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttataggccttgccagggtttcgagaagctgctctggaatttgcaagtcatggctgtgccctcttgatgtggtgaggcaggctttgtttatagcttgcgcattctcatggagaagttatcagacactctggctggtttggaatgcttcttctcaggcatgaacatgtactgttctcttcttgaacactcatgctgaaagcccaagtagggggtctaaaatttttaaggactttccttcctccatctccaagaatgctgaaaccaagggggatgacatgtgactcctatgatctcaggttctccttgattgggactggggctgaaggttgaagaggtgagcacggccaacaaagctgttgatggtaggtggcacactgggtgcccaagctcagaaggctcttctgactactgggcaaagagtgtagatcagagcagcagtgaaaacaagtgctggcaccaccaggcacctcacagaaatgagatcaggctctgcctcaccttggggcttgacttttgtataggtagatgttcagattgctttgattaatccagaataactagcaccagggactatgaatgggcaaaactgaattataagaggctgataattccagtggtccatggaatgcttgaaaaatgtgcaaaacagcgtttaagactgtaatgaatctaagcagcatttctgaagtggactctttggtggctttgcattttaaaaatgaaattttccaatgtctgccacacaaacgtatgtaaatgtatatacccacacacatacacacatatgtcatatattactagcatatgagtttcatagctaagaaataaaactgttaaagtctccaaa

SEQ ID NO: 107 - DNA, 1968 bp, Homo sapiens
  human CXCR6 transcript 1 NM_006564.2
acatcacagaagtagaggcagaccttgcttcatgagcaagctcatctctggaacaaactggcaaagcatctctgctggtgttcatcagaacagacaccatggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttataggccttgccagggtttcgagaagctgctctggaatttgcaagtcatggctgtgccctcttgatgtggtgaggcaggctttgtttatagcttgcgcattctcatggagaagttatcagacactctggctggtttggaatgcttcttctcaggcatgaacatgtactgttctcttcttgaacactcatgctgaaagcccaagtagggggtctaaaatttttaaggactttccttcctccatctccaagaatgctgaaaccaagggggatgacatgtgactcctatgatctcaggttctccttgattgggactggggctgaaggttgaagaggtgagcacggccaacaaagctgttgatggtaggtggcacactgggtgcccaagctcagaaggctcttctgactactgggcaaagagtgtagatcagagcagcagtgaaaacaagtgctggcaccaccaggcacctcacagaaatgagatcaggctctgcctcaccttggggcttgacttttgtataggtagatgttcagattgctttgattaatccagaataactagcaccagggactatgaatgggcaaaactgaattataagaggctgataattccagtggtccatggaatgcttgaaaaatgtgcaaaacagcgtttaagactgtaatgaatctaagcagcatttctgaagtggactctttggtggctttgcattttaaaaatgaaattttccaatgtctgccacacaaacgtatgtaaatgtatatacccacacacatacacacatatgtcatatattactagcatatgagtttcatagctaagaaataaaactgttaaagtctccaaa

SEQ ID NO: 108 - DNA, 2025 bp, Homo sapiens
  human CXCR6 transcript 2 NM_001386435.1
agcatccttccttgctagagagaaagggcactttggtccctggaaagacagaggcaagcagcagcatcggagacactgctcccagtcaggactcaaagtcagcgacagaagtgtttctgagtggattaggaaaggtgttcatcagaacagacaccatggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttataggccttgccagggtttcgagaagctgctctggaatttgcaagtcatggctgtgccctcttgatgtggtgaggcaggctttgtttatagcttgcgcattctcatggagaagttatcagacactctggctggtttggaatgcttcttctcaggcatgaacatgtactgttctcttcttgaacactcatgctgaaagcccaagtagggggtctaaaatttttaaggactttccttcctccatctccaagaatgctgaaaccaagggggatgacatgtgactcctatgatctcaggttctccttgattgggactggggctgaaggttgaagaggtgagcacggccaacaaagctgttgatggtaggtggcacactgggtgcccaagctcagaaggctcttctgactactgggcaaagagtgtagatcagagcagcagtgaaaacaagtgctggcaccaccaggcacctcacagaaatgagatcaggctctgcctcaccttggggcttgacttttgtataggtagatgttcagattgctttgattaatccagaataactagcaccagggactatgaatgggcaaaactgaattataagaggctgataattccagtggtccatggaatgcttgaaaaatgtgcaaaacagcgtttaagactgtaatgaatctaagcagcatttctgaagtggactctttggtggctttgcattttaaaaatgaaattttccaatgtctgccacacaaacgtatgtaaatgtatatacccacacacatacacacatatgtcatatattactagcatatgagtttcatagctaagaaataaaactgttaaagtctccaaa

SEQ ID NO: 109 - DNA, 2831 bp, Mus musculus
  mouse Ccr1 NM_009912.4
atgagtcctaggaacagctcattgtcacacacagagttcaactcaactccatccaacccctccctacacaattcttgtagggcagagctacctctgagaagactggaactttactggaactggagcagagaggaaagacagaacactttagtgaacttggaccccaggaattggccactggtgagcactgtagtgcttcataggcagccaaccataaaaaagaagaactttacaccattcttgtcattctgaaagactataaaaccagagtctaatctccatcatcatacaggaagccaagaacttcctactaggttgggaccttgaaccttgaattcataaagatcctcaaaggcccagaaacaaagtctgtgtggacaaaatactctggaaacacagactcactgtctttaacagaaaagtaaattcagaagctattattcctgaccagttcctcagcaaaggatggagatttcagatttcacagaagcctaccccacaactacagaatttgactatggggactccactccatgccaaaagactgctgtaagagcctttggggctggactcctgccccccctgtattctctagtgttcatcattggagtggtgggcaatgtcctagtgattctggtgctcatgcagcataggaggcttcaaagcatgaccagcatctacctgttcaacctggctgtctctgatctggtcttccttttcactttacctttctggattgactacaagttgaaagacgactggatttttggtgatgccatgtgcaagcttctctctgggttttattacctgggtttatacagtgagatcttctttatcatcctgttgacgattgacagatacctggccattgtccatgctgtgtttgccctgagggcccgaactgttacttttggcatcatcaccagtattatcacctgggccctagccatcttagcttccatgcctgccttatacttttttaaggcccagtgggagttcactcaccgtacctgtagccctcatttcccctacaagagcctgaagcagtggaagaggtttcaagctctaaagctaaaccttcttggactaattttgcctctgttagtcatgataatctgctatgcagggatcatcagaattctgctcagaagacccagtgagaagaaggtcaaagccgtgcgtctgatatttgctattactcttctattcttcctcctctggaccccctacaatctgagtgtatttgtttctgctttccaagatgttctattcaccaatcagtgtgagcagagtaagcaactggacctggccatgcaggtgactgaggtgattgcctacacccactgttgtgtcaacccaatcatttatgtttttgtgggtgaacggttctggaagtaccttcggcagctgtttcaaaggcatgtggctataccactggcaaaatggctgcccttcctctctgtggaccaactagaaaggaccagttctatatctccatccacaggagaacatgagctctctgctggcttctgattcagaccataggtgtcaaccaaggaaggtctaagaagagaatgaggagacagtatatagctctccaagactgatactgacagttcttacagtcaggattcccaatcttttagggtcaaaggaaacttagaggtagcttaggtataagtcacctctttaattgtttccttgaacttctctctgttagaaaggagatgaataaacaaaatggggcattccagaaactggaataaaggtgacaatgagtggcatgggcccaataagagataggatctgtggttattaatttaaaaacaaagccactacttttactgaattcaattcctaatgatgttatattctgaaaaaaattaaagcattgtaatagagaagaattttgcatatttgcatattgacatatattataaagtataagatgttatgtcaaaaaattaaatctgtaacaaactaaagtttttctatccaattattttaaataattagcaaagatgcaaatagatcagaggataaatgcaatagcattgatatgaaaatactgatcaaaaggaaatgacaattaaaatataaaagttcttttggatgctgaaagtagtgaataaatattatttcattttactaacaaaaatgcacattacaggtccagatatggcaaaaacagtcactgacttgcagtttgatacaaatgtaaattagtgcagtatttctgaaaaggaatttgaaaagttcaagaatactcaaacaattttagggtgatattaacaaatgctattaaaatgtcatatagtttaataatttcagaaatattaaatgtcagtcaatatggtatattttaaagaatttttattgatctgggaatatgttcataatattactaaattaaaatggaaaattaaaaactggcaaatggctcactggaaaaggcacttaccaccaaccttaacaacctgagttcaattttaggaactcacatgatgaaaggaaagaactgattctaacaaatagtcctttgatctctaaatgtgcacatatggagacatacacccaaataaattaatgcaaaaaatatagaattcttttggcccatattttatatatgaaaagaacaaactaaaagaatacatcaatccattttcctctgctataataaaatccccaaagctgatactttataaagaataaaggtttgtttagcttgaaactttggagactcagcatgacattctgctcagctctcataagagctccatcagtgcatcactgcacggaataggcttcatgatctgagcacaggcaagaag

SEQ ID NO: 110 - DNA, 3343 bp, Mus musculus
  mouse Ccr3 transcript X1 XM_017313119.3
gttcatcttgatatgtcaccagcctcttgttgatgtctcactgaatcgagagataaaaacctctctggaagtgaatggagtaaagtagtcgcagtggtaccacagacttccatcttctggaacagaccttccagtggttccgaattctctgagagatggagcttctgaatcaacatcttcctaatggatggatggattaatgggtatatagacagcagtgaaacttaagttactactggactcataaaggacttagcaaaattcaccagagacaagtagaatggcattcaacacagatgaaatcaagactgtggttgaaagctttgagaccacaccctatgaatatgagtgggcaccaccctgtgaaaaagtcagaatcaaagagctggggtcatggctcctgcctccactgtactccctggtgttcatcatcggcctcctgggcaacatgatggttgtgttgatcctcataaagtacaggaagctacaaattatgactaatatctacctgttcaacttggcaatttctgacctgctctttctcttcactgtcccattctggattcactatgttctgtggaatgagtggggttttggccactacatgtgcaaaatgctgtctgggttttattacctggccttgtacagcgagatctttttcatcatcctgctgacaattgacagatacctggctatcgtccatgctgtgtttgcccttcgagcccgaactgtgacttttgctactatcaccagtatcattacctggggccttgcaggactggcagcattgcctgaatttatcttccatgagtctcaagacagctttggagagttttcctgcagtcctcgctatccagagggtgaagaagacagctggaaacgtttccatgctctaagaatgaatatctttggtctagctcttcctctcctcattatggttatctgctactcaggaatcattaaaactctgctgagatgtcccaataaaaaaaaacacaaggccatccgtcttatttttgttgttatgatagtcttttttattttttggaccccgtacaacctggttctccttttttctgcttttcacagcacatttttagagaccagctgtcagcagagtaaacatctggacctggccatgcaggtgactgaggtgattgcctacacccactgctgtattaatccagtaatctacgcctttgttggtgagaggttccggaaacaccttcggctctttttccacagaaatgtggcagtttacctgggaaaatatattccgtttcttcctggtgagaaaatggaaagaacaagctctgtctccccatcaactggggagcaagaaatctctgtggtgttttagttgggcagagaaaattgtcacttattccatggactgaaagatgaagcaaacacattaagtcagtcacaatgacctctagctcagtcatttgtactctccatgcaatgctgatgctctcaaagacctgtaaacacacataataactgcagaatatcttcatctaccccaagatcattagtagaggacatgtgctggccagttgacaattcatcaaacatgacatgaaaaagacagctttttttttttattctccaaagagttatacatgttctgattcacttaaatgttaaataatccattcacaagtaggaaggtttaaaaaatatttaaactacttttgccactttttgacatcagtgaaatgtttcacaaaatgcatttattttatttcctaacgtaagtagttctttttcttcttgatgaatggctttctttttcctctttttaaaaaaattattatttgaatatagactggatgttatatcttgtttaattttttcttaaaagtcaaatgaaatcatatttcctattagtgattgaaagcatcttggaaggaacacactctcctcataatgccagttttatgttgaaaaacaggctggggccatctgtattgagaagtccaggtgtggtgctgtcgttaattcttcctagacttgacatcataagtacctggagggtatagtgacacatgcacaggtaactcctattacaggagaagagtagaaatttggaaacagacatcaagattaacagatgaagtaagactggaaacccctttctaccggccctcacataccaaatagaggatgtacaagaaaagtatatgatatcacacagaaagaagttggcactatgcaaataacccatgaatgataagaattcatatgatgcagtgcttttgggtgtttgtcttttcacagatcattagcctacttctatcctcaagaatatctttttatctttcttaataaactgcctgtgtttctatcattatctatatgttcattattctggcacacagaccctagaaatctcaggaggtgctctctggattgaagtgtgcacttatagcaccacctcagtgcttatgactcatgtttgaagtgaggtctgagcatcaacaacacgttcccaggtggtggacgtgatacttgtcccaagatcacactttcagaattatgttgtaactgtgatgcgttcagagactcaacaattgaaaatgaggatgtcacaagtgttgtaggaggaatatatgggctctttagatgtttgataaggttatatgctatcatggtcagctgataagatatgctagtctatctgaaatattactatagggggaacttacagttctatcagtctatcagttttatacagttgagcttttcttgagtatatgtgtttagtactatgaactagaagctgtgagagacacaaagatgaaggaggaaaatggagtggctatggctcaggcacctttctctcattcctttacttctgattatagcttcttggggatgtaacttctaagcctttacttaacttggcacttttcttttgggtatagcatagtcatctcatgattattttgaagtagagattaccttggtctagtaaccatgacaactatttgagcagaacaatgaagaggtaagttagtcagtagtgatgctattggagttactgatactccatagtagaaggaaatatagggtcatagtcccctcatcttgtattacaaccattactctctccttctccaaattgctcatgatcttgggagatgataaagatatataagattggaagagttacttgagagtgtgactccatgatttcattttcatgaggtcattatccataatgggatgagattttttgtgatgagacagctgtttgagggtaactcactcttttaagtatcccttcaataatgctctgtaagtaggctcaataaactcattggtttgctaagttgaa

SEQ ID NO: 111 - DNA, 3273 bp, Mus musculus
  mouse Ccr3 NM_009914.4
agtaaataacaaaacggaagaactgtaacaagttggtatctctctgaaaacttgcaaaacctgagaagctagcctttttaaaacaggaagctatgatgtttactacctgactggtgtatatgatcaagtgttatctctgtttcattagcagtgaaacttaagttactactggactcataaaggacttagcaaaattcaccagagacaagtagaatggcattcaacacagatgaaatcaagactgtggttgaaagctttgagaccacaccctatgaatatgagtgggcaccaccctgtgaaaaagtcagaatcaaagagctggggtcatggctcctgcctccactgtactccctggtgttcatcatcggcctcctgggcaacatgatggttgtgttgatcctcataaagtacaggaagctacaaattatgactaatatctacctgttcaacttggcaatttctgacctgctctttctcttcactgtcccattctggattcactatgttctgtggaatgagtggggttttggccactacatgtgcaaaatgctgtctgggttttattacctggccttgtacagcgagatctttttcatcatcctgctgacaattgacagatacctggctatcgtccatgctgtgtttgcccttcgagcccgaactgtgacttttgctactatcaccagtatcattacctggggccttgcaggactggcagcattgcctgaatttatcttccatgagtctcaagacagctttggagagttttcctgcagtcctcgctatccagagggtgaagaagacagctggaaacgtttccatgctctaagaatgaatatctttggtctagctcttcctctcctcattatggttatctgctactcaggaatcattaaaactctgctgagatgtcccaataaaaaaaaacacaaggccatccgtcttatttttgttgttatgatagtcttttttattttttggaccccgtacaacctggttctccttttttctgcttttcacagcacatttttagagaccagctgtcagcagagtaaacatctggacctggccatgcaggtgactgaggtgattgcctacacccactgctgtattaatccagtaatctacgcctttgttggtgagaggttccggaaacaccttcggctctttttccacagaaatgtggcagtttacctgggaaaatatattccgtttcttcctggtgagaaaatggaaagaacaagctctgtctccccatcaactggggagcaagaaatctctgtggtgttttagttgggcagagaaaattgtcacttattccatggactgaaagatgaagcaaacacattaagtcagtcacaatgacctctagctcagtcatttgtactctccatgcaatgctgatgctctcaaagacctgtaaacacacataataactgcagaatatcttcatctaccccaagatcattagtagaggacatgtgctggccagttgacaattcatcaaacatgacatgaaaaagacagctttttttttttattctccaaagagttatacatgttctgattcacttaaatgttaaataatccattcacaagtaggaaggtttaaaaaatatttaaactacttttgccactttttgacatcagtgaaatgtttcacaaaatgcatttattttatttcctaacgtaagtagttctttttcttcttgatgaatggctttctttttcctctttttaaaaaaattattatttgaatatagactggatgttatatcttgtttaattttttcttaaaagtcaaatgaaatcatatttcctattagtgattgaaagcatcttggaaggaacacactctcctcataatgccagttttatgttgaaaaacaggctggggccatctgtattgagaagtccaggtgtggtgctgtcgttaattcttcctagacttgacatcataagtacctggagggtatagtgacacatgcacaggtaactcctattacaggagaagagtagaaatttggaaacagacatcaagattaacagatgaagtaagactggaaacccctttctaccggccctcacataccaaatagaggatgtacaagaaaagtatatgatatcacacagaaagaagttggcactatgcaaataacccatgaatgataagaattcatatgatgcagtgcttttgggtgtttgtcttttcacagatcattagcctacttctatcctcaagaatatctttttatctttcttaataaactgcctgtgtttctatcattatctatatgttcattattctggcacacagaccctagaaatctcaggaggtgctctctggattgaagtgtgcacttatagcaccacctcagtgcttatgactcatgtttgaagtgaggtctgagcatcaacaacacgttcccaggtggtggacgtgatacttgtcccaagatcacactttcagaattatgttgtaactgtgatgcgttcagagactcaacaattgaaaatgaggatgtcacaagtgttgtaggaggaatatatgggctctttagatgtttgataaggttatatgctatcatggtcagctgataagatatgctagtctatctgaaatattactatagggggaacttacagttctatcagtctatcagttttatacagttgagcttttcttgagtatatgtgtttagtactatgaactagaagctgtgagagacacaaagatgaaggaggaaaatggagtggctatggctcaggcacctttctctcattcctttacttctgattatagcttcttggggatgtaacttctaagcctttacttaacttggcacttttcttttgggtatagcatagtcatctcatgattattttgaagtagagattaccttggtctagtaaccatgacaactatttgagcagaacaatgaagaggtaagttagtcagtagtgatgctattggagttactgatactccatagtagaaggaaatatagggtcatagtcccctcatcttgtattacaaccattactctctccttctccaaattgctcatgatcttgggagatgataaagatatataagattggaagagttacttgagagtgtgactccatgatttcattttcatgaggtcattatccataatgggatgagattttttgtgatgagacagctgtttgagggtaactcactcttttaagtatcccttcaataatgctctgtaagtaggctcaataaactcattggtttgctaagtt

SEQ ID NO: 112 - DNA, 6579 bp, Mus musculus
  mouse Ccr5 transcript X1 XM_036154589.1
ctccaccctctctgcaagaaactgtgtttcttactttatactgtcttcatgttagatttgtacagctctcctagccagaggaggtgagacatccgttccccctacaagagactctggctcttgcaggatggattttcaagggtcagttccgacctatagctatgacatcgattatggtatgtcagcaccctgccaaaaaatcaatgtgaaacaaattgcggctcagctcctgcccccactctactccctggtattcatctttggttttgtgggtaacatgatggtcttcctcatcttgataagctgcaaaaagctgaagagcgtgactgatatctacctgctcaacctggccatctctgacctgctcttcctgctcacactaccattctgggctcactatgctgcaaatgagtgggtctttgggaacataatgtgtaaagtattcacagggctctatcacattggttattttggtggaatcttcttcattatcctcctgacaattgataggtacttggctattgtccatgctgtgtttgctttaaaagtcagaacggtcaactttggggtgataacaagtgtagtcacttgggcggtggctgtgtttgcctctctcccagaaataatctttaccagatctcagaaagaaggttttcattatacatgcagtcctcattttccacacactcagtatcatttctggaagagtttccaaacattaaagatggtcatcttgagcctgatcctgcctctacttgtcatggtcatctgctactcaggaattctccacaccctgtttcgctgtaggaatgagaagaagaggcacagggctgtgaggctcatctttgccatcatgattgtctactttctcttctggactccctacaacattgtcctcctcctgaccaccttccaggaattctttggactgaataactgcagtagttctaatagactagaccaggccatgcaggcaacagagactcttggaatgacacactgctgcctaaaccctgtcatctatgcctttgttggagagaagttccggagttatctctcagtgttcttccgaaaacacatggtcaaacgcttttgcaaacggtgttcaattttccagcaagacaatcctgatcgtgcaagctcagtctatacccgatccacaggagaacatgaagtttctactggtttatgacctggttgacttttgtgtatcacgtagtttttctatgcagcttgggagtaggaatggttcttttaaaaaaagaaattagtatcatagagggcccaagatacatgcatctttttgatatttatttttagatagattgggtcttttaaaactgaatggggaggttggggtggaggagcagggagaacgagtcttttatcagggccgggaaatatgcacaaagagacttgaggcaggtgccatgacccatatgcaaagggacggacacagggccgatgctgtggcctagagatgacgtgtctcaccgctgggttcctgaaaggcggctgtaaatatgcctgattgccataaagtcgcttcttgctgtctatggatgtgcctgactgccaacagggaagaaccacttctgcctataaaacgtagagtcagcagaacttggggtaaatcggagttagaggtgcataagaacccctaggcttagttaggttgaaatacccattgaggaaacagcaaatacaaaggaagaataaagagtttagccgggaaggtagtctcattttacagccggaatataatgttatctcaggctagcattttgttcctgccttcagacctaaatcctaccacaccgggactgtgaaacacctggattatgaatcatgagcctgaggtctaggaataataataacgtttgtgattttagatgagggctgtttccatagtttgaagccagaactttatcatcttgagcagaagctccaagagatgaggaaagagcaccaatttttctctaatttacttagcagtcatcatctctggaagattcattttagaaacaagttgttgtgcccctcagaagccatgagagtataacgactgctctctgtgttccaggctgagtatgaggacttcagtcacactttccagatggcttctccacacaaacaatgctaagtttggccatttcagaggtttaggattttttgttgtttttgcagttgatattttgaattttagagcagttgagatcttcctagtgaaggctagaggaggaaagaaaggggttagaatctctcaggagattaaagtttctgcctaacaagaggtgttactggtttttctcaagctccgattgtgaaaccagaggcctgggactgtcagcaggaagtgagcatttgcttttttcttccttgtgatccacattcctcccccactctgttgctcagactggcgtcaagctcacgatcctcctgcttacatctcaagttctgagattacaagtatatgtgaacatatccagcggttattttattcattagcatatagaaagttatacgttctttgaagataatgagtcttataaaaagtgctttgtaaaaaaaattgcattttatactttcaatcaagtgtacatttagtgagtagtacgtaaaattatgagagtattttgtaagtagttgttttggagaacgcccccaataatacttgtttaaatatagcgttcttggattaagtgggtggtggtgatgataatatttccttgaaagtatttttagccgttaactttcttccttaaacaatttttcataataatttgttcttaaagatgttatgtccaagcatgcagtttcggagcagtgttgctttgaaagagtgtaaattttaaattgtgcttactctcaatcaaaagagttttaacatatttacgaatttatttcagaagtcaagaatctggttgaaaataaagatatgcaactttatggtctggtatttgagtgggagtcagagagggtgaggcctttctctctctctctctctctctctctctctctctctctcaagtgtgttaataatttcagtaatggaaattctataacactgggatttctgtgcacatgtttcttgtccagagagaggatagatcctccaaggagagtctggaacaatgctgattttcagtctgctacagccccagcttccaaagagggtgtgcaaagatgttcgggctattaatgctcctaatgaaagagacaaacgagcaggaagtaaaatgggctctgagtggtttagtaacttctcaccagattcaaagggttgcttttcatctcttagatttgaaggtagaaagtgttaaagaagagtggaaggcagctgggcttaggagaaaggagtcaggggaagggagccctctatctgctatgataaagccagcttttgtggggatgagggaaagtgtgttaataacttatcttgtggctgtgacaaaatatctgacaaaggcagcttaaagaagaaagttttacggttgttggtagtttgatggaatacattctaccgtggtgtgttaggagtgtgagcttggtggtaatgtcacatctacatccaggaagcaaagaattgaatgctggtgtcagctctctttctcctctttattcagtccaattccccagtccataaaatggtgttgttcatattcagggtgggtcttctctccttggctaatcctttgtggaaacatcctcataggtacagacagaaatgtttatcctaggtagttctaaatccagtaaagcaatcaatcaatactaaccactacaaaagctctctctctctctctctctccctctctctctctctctttctctctctccctttttttatggctgatgtacacactccttcttaccttgccatatgttagatgaatttacaaagaaaaaccacttgaggtattgtataactatgtatatattttctttcataatttaaaaaatactttaatgttccatttggctccatattgttactttttcgatgaattataatttctgtgttattataattctgtaatacctcaagagcttattttgttttaaaaataggggattttgtttgtctattttgagagtttcttctgtagtccaggctgccctggaacttcctctgtagatcaggctggcctcaaatatgaaatatgcccgcctctgcttccaaattgcttggattaaaggtgtgcactaccattacctggctcaaagatgagttttaaagcatacatagtgaaaaataaccccttttcctgattgagccaagctatcaaaactgcaaataacctaggctatattgattctatactgccctccagtggccaactagcaaataacaaagatgtttggcaaccagcatggaccacatcatacaaataaggagtatggcttcactgaattttttactgtttgtgtctttaattccagtgtggacagaggcatatccagagcagctcagggtatttgacacacagctctttctcactgcttctgaagaacaggaagagccccagttgaggatacaagttgacactttgaaataatggtttcaggtggatctgcagaatgctggagcccagaccttgaaaaggctcgggaaattacttataaacaggtattttcacatgcatgaagtgccccagactctccttgcatttcactctaaccctgcatatcccaagtgtttgcagtgttccaatatagttgaaaggcaggtggctttctttgattctttttattctcttttcttcattaccattattattccatccacacggatgaaacattcttacaaaactatacacctttaaaatggaaaagctgttgaggatcactcagtccaggtgcctctctctgtaggtgaaaagcagagtctactccacactctcaaagagggtttcagaggactcaagaattagcaagttttgacagtgttttccaatctcagtgtgcagtcggtgataagccattgaacgagtgggtggggagagggtctgataccacttacaatcatgcctagataaattcaggagatgagtttacaattaatgaaggctacattctagtcctggcttcatcttagctacactttgaaatgacacgggaagttgacaccaagtctcactacaattctacactgaacaactctctttaatcaacccacatgtacctgtgtttctaactcttaggaattccccaggtggttctcaaaatagccaagtttgagacactggtagtgaaaactgggttgaagtctgtcatactggatatgatgtttggaatctgagctttgtcacacagtgtgagggtttggagaatgaagaagatcatgacctggtcaggcaaggttaattcggaaaaagttgaaaagttagcattcagagagtaacttttagatggcctggcacctaatttaaatgtgtgcacatatatgcatgaaacaaagcttggcttgcattagttacttttgtgtctctgtgaccatagtactcagctaaaacaactgaaggaaagagtttactttggctcatagctttagttcattatggggaagtcatagcattgtatcaaagtacatgggtatgtataccagagactgttcatgttacagacgtccagaaagcagagtccagaagcaggggcaagctataactttcaaaggatcatccctagtgccttcaagccagccagacttcacctcctaaagaatctatgggcttaccaatagtgccagtctggacatattttgctatacacaaaacatgaccctgtgagggatatttctgaaagacagatataagttgctctttttgttttgagtcaagacaacatcccctcttgaaactaagtatcactgaacataatgcagctaagtttaaaaataaaaacatcgtggaaatgcttcaggatagtctatatttaccagaaaattctaccaggatttttctacttacatttatttccattatcttagattactcaaaaaatatcctataaagaagttataatgttttctcaatatataatcaaatcaatcatctgttaatgggacagctcagtctttcctgcatggtaaaaaccaaactctgggatgagaaggggacttgagattacttgcatcagtttatgagttctaaaaaaatcattatcaggaatttgaaatcatcatctcatctacacagaatttctacatgaaagtacatggtcagattgattctacaggaaatctgctcaacagagtgggctgatgatcaggaatcattaggctacgtaatagtggcaagataggactattagaagcaagaagcaatgctgggatgaagtctctgaggaccctgcaacccagaacacacccatcaacagctttgtaaaatatagggccatctcaagaaaactcacttgctgaagcctttcatagatggcatctgacactagaaatcattcttagtttgtatatgcataagattgggttaaatctcttacacaaagtataaataaaagtatatctgtagaaaatagttggatatccgatgggaattgtgtataaacatggaaacaacctca

SEQ ID NO: 113 - DNA, 2926 bp, Mus musculus
  mouse Ccr5 NM_009917.5 Ccr5
gtgtttcttactttatactgtcttcatgttagatttgtacagctctcctagccagaggaggtgagacatccgttccccctacaagagactctggctcttgcaggatggattttcaagggtcagttccgacctatagctatgacatcgattatggtatgtcagcaccctgccaaaaaatcaatgtgaaacaaattgcggctcagctcctgcccccactctactccctggtattcatctttggttttgtgggtaacatgatggtcttcctcatcttgataagctgcaaaaagctgaagagcgtgactgatatctacctgctcaacctggccatctctgacctgctcttcctgctcacactaccattctgggctcactatgctgcaaatgagtgggtctttgggaacataatgtgtaaagtattcacagggctctatcacattggttattttggtggaatcttcttcattatcctcctgacaattgataggtacttggctattgtccatgctgtgtttgctttaaaagtcagaacggtcaactttggggtgataacaagtgtagtcacttgggcggtggctgtgtttgcctctctcccagaaataatctttaccagatctcagaaagaaggttttcattatacatgcagtcctcattttccacacactcagtatcatttctggaagagtttccaaacattaaagatggtcatcttgagcctgatcctgcctctacttgtcatggtcatctgctactcaggaattctccacaccctgtttcgctgtaggaatgagaagaagaggcacagggctgtgaggctcatctttgccatcatgattgtctactttctcttctggactccctacaacattgtcctcctcctgaccaccttccaggaattctttggactgaataactgcagtagttctaatagactagaccaggccatgcaggcaacagagactcttggaatgacacactgctgcctaaaccctgtcatctatgcctttgttggagagaagttccggagttatctctcagtgttcttccgaaaacacatggtcaaacgcttttgcaaacggtgttcaattttccagcaagacaatcctgatcgtgcaagctcagtctatacccgatccacaggagaacatgaagtttctactggtttatgacctggttgacttttgtgtatcacgtagtttttctatgcagcttgggagtaggaatggttcttttaaaaaaagaaattagtatcatagagggcccaagatacatgcatctttttgatatttatttttagatagattgggtcttttaaaactgaatggggaggttggggtggaggagcagggagaacgagtcttttatcagggccgggaaatatgcacaaagagacttgaggcaggtgccatgacccatatgcaaagggacggacacagggccgatgctgtggcctagagatgacgtgtctcaccgctgggttcctgaaaggcggctgtaaatatgcctgattgccataaagtcgcttcttgctgtctatggatgtgcctgactgccaacagggaagaaccacttctgcctataaaacgtagagtcagcagaacttggggtaaatcggagttagaggtgcataagaacccctaggcttagttaggttgaaatacccattgaggaaacagcaaatacaaaggaagaataaagagtttagccgggaaggtagtctcattttacagccggaatataatgttatctcaggctagcattttgttcctgccttcagacctaaatcctaccacaccgggactgtgaaacacctggattatgaatcatgagcctgaggtctaggaataataataacgtttgtgattttagatgagggctgtttccatagtttgaagccagaactttatcatcttgagcagaagctccaagagatgaggaaagagcaccaatttttctctaatttacttagcagtcatcatctctggaagattcattttagaaacaagttgttgtgcccctcagaagccatgagagtataacgactgctctctgtgttccaggctgagtatgaggacttcagtcacactttccagatggcttctccacacaaacaatgctaagtttggccatttcagaggtttaggattttttgttgtttttgcagttgatattttgaattttagagcagttgagatcttcctagtgaaggctagaggaggaaagaaaggggttagaatctctcaggagattaaagtttctgcctaacaagaggtgttactggtttttctcaagctccgattgtgaaaccagaggcctgggactgtcagcaggaagtgagcatttgcttttttcttccttgtgatccacattcctcccccactctgttgctcagactggcgtcaagctcacgatcctcctgcttacatctcaagttctgagattacaagtatatgtgaacatatccagcggttattttattcattagcatatagaaagttatacgttctttgaagataatgagtcttataaaaagtgctttgtaaaaaaaattgcattttatactttcaatcaagtgtacatttagtgagtagtacgtaaaattatgagagtattttgtaagtagttgttttggagaacgcccccaataatacttgtttaaatatagcgttcttggattaagtgggtggtggtgatgataatatttccttgaaagtatttttagccgttaactttcttccttaaacaatttttcataataatttgttcttaaagatgttatgtccaagcatgcagtttcggagcagtgttgctttgaaagagtgtaaattttaaattgtgcttactctcaatcaaaagagttttaacatatttacgaatttatttcagaagtcaagaatctggttgaaaataaagatatgcaacttta

SEQ ID NO: 114 - DNA, 3043 bp, Mus musculus
  mouse Cxcr2 NM_009909.3
agtagtttcctcatcacggctgcctcactttcttccagttcaaccagccctgacagctcccaagccttgagtcacagagagttgggagccactctgctcacaaacagcgtcgtagaactactgcaggattaagtttacctcaaagatgggagaattcaaggtggataagttcaacattgaagatttcttcagtggagatcttgatattttcaattatagctctggcatgccctctattctgccagatgctgtcccatgccactcagagaacctggaaatcaacagttatgctgtggttgtaatatacgtcctggtgactctgctgagccttgtggggaactccttggtgatgctggtcatcttatacaaccggagcacctgctctgtcaccgatgtctacctgctgaacctggccattgctgacctgttctttgccctgaccttgcctgtctgggctgcatctaaagtaaatggatggacttttggctcaaccctgtgcaagatattctcatacgtgaaggaggttaccttctacagcagtgttctgctactagcctgcatcagcatggaccgctacctggccattgtacatgccacaagtacactgatccagaagagacacttggtcaagtttgtgtgcatagccatgtggttactatcagtaattctggccctgcccatcttaattctacgaaatcctgttaaggtaaacctttctaccttagtctgctatgaggatgtaggtaacaatacatcccgtttgagggtcgtactgcgtatcctgcctcagacttttggcttcctcgtgccgctgctcatcatgctgttctgctacgggttcacactgcgcaccctctttaaggcccacatggggcagaagcaccgggccatgcgggtcatcttcgctgtcgtccttgtcttcctgctctgctggctgccctacaacctggttctgttcacagacaccctcatgagaaccaagctgatcaaggagacctgtgagcgccgcgatgacattgacaaggccttgaatgctacggagattcttggcttcctccacagctgccttaaccccatcatctatgcctttattggccagaaatttcgccatggacttctcaagatcatggctacttatggccttgtcagcaaggagttcttagccaaggagggaaggccttcttttgttagctcgtcttcagcaaacacctctactaccctctaaagactgttcacctaaacggtggcccctcggggttccttcttgtctttcagcatggctcattaccagagactgtggtatttgaattgatgcagcccctcctctacaattacagggagaaagaggtcacgttcttagcagagcccccagagtttagaaccccctatattggctgtctgctcccttccatcttggtatgcctactgatagagttgatccatcctaacactagaccccaaacactcttttctaagaagcacacgttacaattacagtgagatactgcctcctacccatcagaacagttagcagtaaaaggaagaggtggaggagagaaaatggcaagtgacaatgaggaagtagaaaaagggaactcttcaccttaccagtagacgagtaccagagtcccctcacacaggaacatagcatagcagttttccatttaaaaaaaaattaagggctggtgagatggctcagtgggtaagagcacccgactgctcttccaaaggtccgaagttcaaatcccagcaaccacatggtggctcacaaccatctgtaacgagatctgatgccctcttctggagtgtctgaagacagctacagtgtacttacatataataaataaataaatctttaaaaaaaaaaattaaatatagaattggcatgtaatccagcaagtccatctctaagtagggtacctaaaaaaattgaaagccagggttcagacatttgtatgctgatactcacaacagcattattcatcatgaaaatgatggccaacagatgaatgggtaagttaaacaaggtgtctctacacaacaaaagagcgtccacaatgagaagtgccaaaaataatcaaatagtgttgactggaggctgggtgaggaagggtggggagttcgtgtagaagacacagctccagttagggatgatgggaaagccctgaaggtgggccctgatccctgtgtagtagtgtgaatgcactcattaccactgtaacaaaagtgttaaaatgaaagctgagtattgctccctgggagagtgcttatttaacatttctaagaactcaggctcaaatcctagcacctcgacaaacaaacaaacaaacaaacaaaaaagctaaaatggttatttttgttatgtgtgttttacctcactttaccaaaaatataaaaatctaagtctaaaaaatgttcaccatcccataaacaaccaccaaacccagacactaggcagatgccaacaagaacctgctgacaggaggctgatatagctgtctcctgaggagctctgccagtgcctggaaaatacagaagtggatgctcacaatcatccattggatagaggacaaggtcccaaatgaagaagctagcgaaggtatccaaggagctaaaggggtctgaagccccagaggaggaacatcagtatgaactagccagtacccccccagagctccttggaactaaaccaccaatcaaagaaaacacatggtggaacttgtggctctagctgtatatgtagcagaggatggcctagtcagtcatcaatgggaggagaggcccttggtcctgtgaaggctctatgcctcaatataggggaatgccaggaccaggaatgggagtaggtggattggggagcagggggagagggaagggaataggggatatttggaggggaaactaggaaaggggataacatttgaaatgtaaataaagaaaatatctaataaaaaaaatgttctttaaccactgaaccaatgcagaactatctaccttataatttcttgtaaagtgtgactattaaatgtcgttttaaaaaata

SEQ ID NO: 115 - DNA, 2995 bp, Mus musculus
  mouse Cxcr2 XM_006495638.5
cctcttaagaatttcactgctcttcatgggtcacagctgcctaccttaggtgtcccacaggtgaaaagccagcatcctggattaagtttacctcaaagatgggagaattcaaggtggataagttcaacattgaagatttcttcagtggagatcttgatattttcaattatagctctggcatgccctctattctgccagatgctgtcccatgccactcagagaacctggaaatcaacagttatgctgtggttgtaatatacgtcctggtgactctgctgagccttgtggggaactccttggtgatgctggtcatcttatacaaccggagcacctgctctgtcaccgatgtctacctgctgaacctggccattgctgacctgttctttgccctgaccttgcctgtctgggctgcatctaaagtaaatggatggacttttggctcaaccctgtgcaagatattctcatacgtgaaggaggttaccttctacagcagtgttctgctactagcctgcatcagcatggaccgctacctggccattgtacatgccacaagtacactgatccagaagagacacttggtcaagtttgtgtgcatagccatgtggttactatcagtaattctggccctgcccatcttaattctacgaaatcctgttaaggtaaacctttctaccttagtctgctatgaggatgtaggtaacaatacatcccgtttgagggtcgtactgcgtatcctgcctcagacttttggcttcctcgtgccgctgctcatcatgctgttctgctacgggttcacactgcgcaccctctttaaggcccacatggggcagaagcaccgggccatgcgggtcatcttcgctgtcgtccttgtcttcctgctctgctggctgccctacaacctggttctgttcacagacaccctcatgagaaccaagctgatcaaggagacctgtgagcgccgcgatgacattgacaaggccttgaatgctacggagattcttggcttcctccacagctgccttaaccccatcatctatgcctttattggccagaaatttcgccatggacttctcaagatcatggctacttatggccttgtcagcaaggagttcttagccaaggagggaaggccttcttttgttagctcgtcttcagcaaacacctctactaccctctaaagactgttcacctaaacggtggcccctcggggttccttcttgtctttcagcatggctcattaccagagactgtggtatttgaattgatgcagcccctcctctacaattacagggagaaagaggtcacgttcttagcagagcccccagagtttagaaccccctatattggctgtctgctcccttccatcttggtatgcctactgatagagttgatccatcctaacactagaccccaaacactcttttctaagaagcacacgttacaattacagtgagatactgcctcctacccatcagaacagttagcagtaaaaggaagaggtggaggagagaaaatggcaagtgacaatgaggaagtagaaaaagggaactcttcaccttaccagtagacgagtaccagagtcccctcacacaggaacatagcatagcagttttccatttaaaaaaaaattaagggctggtgagatggctcagtgggtaagagcacccgactgctcttccaaaggtccgaagttcaaatcccagcaaccacatggtggctcacaaccatctgtaacgagatctgatgccctcttctggagtgtctgaagacagctacagtgtacttacatataataaataaataaatctttaaaaaaaaaaattaaatatagaattggcatgtaatccagcaagtccatctctaagtagggtacctaaaaaaattgaaagccagggttcagacatttgtatgctgatactcacaacagcattattcatcatgaaaatgatggccaacagatgaatgggtaagttaaacaaggtgtctctacacaacaaaagagcgtccacaatgagaagtgccaaaaataatcaaatagtgttgactggaggctgggtgaggaagggtggggagttcgtgtagaagacacagctccagttagggatgatgggaaagccctgaaggtgggccctgatccctgtgtagtagtgtgaatgcactcattaccactgtaacaaaagtgttaaaatgaaagctgagtattgctccctgggagagtgcttatttaacatttctaagaactcaggctcaaatcctagcacctcgacaaacaaacaaacaaacaaacaaaaaagctaaaatggttatttttgttatgtgtgttttacctcactttaccaaaaatataaaaatctaagtctaaaaaatgttcaccatcccataaacaaccaccaaacccagacactaggcagatgccaacaagaacctgctgacaggaggctgatatagctgtctcctgaggagctctgccagtgcctggaaaatacagaagtggatgctcacaatcatccattggatagaggacaaggtcccaaatgaagaagctagcgaaggtatccaaggagctaaaggggtctgaagccccagaggaggaacatcagtatgaactagccagtacccccccagagctccttggaactaaaccaccaatcaaagaaaacacatggtggaacttgtggctctagctgtatatgtagcagaggatggcctagtcagtcatcaatgggaggagaggcccttggtcctgtgaaggctctatgcctcaatataggggaatgccaggaccaggaatgggagtaggtggattggggagcagggggagagggaagggaataggggatatttggaggggaaactaggaaaggggataacatttgaaatgtaaataaagaaaatatctaataaaaaaaatgttctttaaccactgaaccaatgcagaactatctaccttataatttcttgtaaagtgtgactattaaatgtcgttttaaaaaat

SEQ ID NO: 116 - DNA, 1893 bp, Mus musculus
  mouse Cxcr6 NM_030712.4
agagaaccagaggcagacctttagtgagcacacttcactctggaacaaagctactgggcttctcttctgatgccatggatgatgggcatcaagagtcagctctgtacgatgggcactacgagggagatttctggctcttcaacaattccagtgataacagccaggagaacaaacgcttcctaaagttcaaggaggtctttttgccctgtgtgtacctggtagtgtttgtctttggactgctaggaaactccctggttctgattatatacattttctaccagaagctgaggactctgacagatgtgtttctgctgaacttgcccctggctgacctggtgtttgtctgtactctgcccttttgggcctatgcaggcacctatgagtgggtctttggcacagtcatgtgcaaaactcttcgaggcatgtatacaatgaacttctacgtgtccatgctcactctcacctgcatcacagtggatcgtttcattgtagtggtccaggctaccaaggccttcaaccggcaggctaagtggaagatctggggccaagtcatttgcttgctcatttgggtggtctccctgttggtttctttgccacagatcatctatggccatgttcaagatattgacaagcttatctgtcagtaccacagtgaggagatatccactatggttcttgttatacagatgactctggggttcttcctgccattgctcactatgattctgtgctactcaggcattatcaagaccttgcttcatgctcgaaacttccagaagcacaaatctctaaagatcatcttccttgtagtggctgtgttcctgctgacccagacacccttcaaccttgccatgttaatccaaagtacaagctgggagtactataccataaccagctttaagtatgccatcgtagtgacagaggctatagcatactttcgggcttgccttaaccctgtactttatgcctttgttggcttaaagttccggaagaacgtctggaaacttatgaaggatatcggctgcctctctcacctgggagtctcaagtcaatggaagtcttctgaggacagttccaagacttgttctgcctcccacaatgtagagaccaccagtatgttccaattgtagtaggccttgccacacttagagaagttaataacagaattctaggagcatggctgtatcatttggatgcaacaagaaaagctttgcttatagcatgtggagtatcatggagaagtcactgaacaccatggctggtacacaaaacttctcagatataaatataccctattcttaatatctaagcctaatgctcaaaggagaatgagtatcttgagattttgaagcactttctctctttcatcctcaagaatgctgaaatcaaggtcatgacggttaactctagagatctcaagttctctttttaccaagtttgggcctaaaagttgaagagctgcatcaacaaagatgttgctggcagataatgttcagggctcttgagttcagaagattcttcagtgggcaagcagttgagaattgaaaaggctccatacaaatgctgcttgaatgagaccaggttctgactcatccaggggtctgtctgacttttgtatgggtggatgcttatgttttactggaattgtcttgtaaagtgcagcaatatgggactgaaaacaggccagaataaataagctgaatttcagtggtccatgtaatgctagaaaaatgtataaaacagagcttaagactttaacaaatacaagaggactctggtggctttgctcatctaaaaaaaaaaaatgaaagaaattgtgacacatactatgtacacacaaatatatcaagcattttgagtttcatggctaagaaataaaaccttttaaatgtt